(19) United States
(12) Patent Application Publication
(10) Pub. No.: US 2016/0287622 A1
(43) Pub. Date: Oct. 6, 2016

(54) COMPOSITIONS AND METHODS FOR TREATING IMMUNE AND VIRAL DISORDERS AND MODULATING PROTEIN-RNA INTERACTION

(71) Applicant: MASSACHUSETTS INSTITUTE OF TECHNOLOGY, Cambridge, MA (US)

(72) Inventors: Paul CHANG, Cambridge, MA (US); Tanya TODOROVA, Somerville, MA (US); Florian J. BOCK, Boston, MA (US)

(73) Assignee: Massachusetts Institute of Technology, Cambridge, MA (US)

(21) Appl. No.: 15/037,617

(22) PCT Filed: Nov. 7, 2014
(86) PCT No.: PCT/US2014/064570

§ 371 Date: May 18, 2016

Related U.S. Application Data
(60) Provisional application No. 61/905,531, filed on Nov. 18, 2013.
(60) Provisional application No. 61/905,896, filed on Nov. 19, 2013.

(51) Int. Cl. A61K 31/7076; A61K 45/06; G01N 33/573; A61K 31/7088

(52) U.S. Cl. CPC A61K 31/7076; A61K 31/7088; A61K 45/06; G01N 33/573; C12Y 204/0203; G01N 2500/20; G01N 2500/02; G01N 2333/70578; G01N 2333/91142

(57) ABSTRACT

The present invention relates to methods of treating or decreasing the likelihood of developing a disorder associated with immune misregulation, such as, an autoimmune disorder, or viral or virus-associated disorder in a subject including administering to the subject a composition comprising an activator of a CCCH zinc finger-containing PARP, such as, PARP13 or PARP12. The present invention also relates to methods of treating a TRAIL-resistant disorder, such as, TRAIL-resistant cancer including administering to the subject a composition comprising an activator of a CCCH zinc finger-containing PARP, such as, PARP13 or PARP12. The present invention further relates to methods of modulating a CCCH zinc finger-containing PARP-RNA interaction including contacting a CCCH zinc finger-containing PARP protein or a CCCH zinc finger-containing PARP fusion protein with a CCCH zinc finger-containing PARP activator.

### CROSS-REFERENCE TO RELATED APPLICATIONS

This application claims priority to U.S. Provisional Application No. 61/905,531, filed Nov. 18, 2013 and U.S. Provisional Application No. 61/905,896, filed Nov. 19, 2013, each of which is hereby incorporated by reference.

### STATEMENT AS TO FEDERALLY SPONSORED RESEARCH

This invention was funded by grant RO1 GM087465 from the National Institute of Health. The government may have certain rights in the invention.

### BACKGROUND OF THE INVENTION

The present invention relates to the field of molecular biology and molecular medicine.

Poly(ADP-ribose) Polymerase-13 (PARP13), also known as Zinc Finger Antiviral Protein (ZAP), ARTD13, and ZC3HAV1, is a member of the PARP family of proteins—enzymes that modify target proteins with ADP-ribose using nicotinamide adenine dinucleotide (NAD⁺) as substrate. Two PARP13 isoforms are expressed constitutively in human cells: PARP13.1 is targeted to membranes by a C-terminal CaaX motif, whereas PARP13.2 is cytoplasmic. Both proteins are unable to generate ADP-ribose—PARP13.1 contains a PARP domain lacking key amino acid residues required for PARP activity whereas the entire PARP domain is absent in PARP13.2. Both isoforms of PARP13 contain four N-terminal RNA binding CCCH-type Zinc Fingers—domains found in proteins that function in the regulation of RNA stability and splicing such as tristetraprolin (TTP) and muscleblind-like (MBNL1), respectively.

PARP13 was originally identified in a screen for antiviral factors. It binds RNAs of viral origin during infection and targets them for degradation via the cellular mRNA decay machinery. Several RNA viruses, including MLV, SINV, HIV and EBV as well as the RNA intermediate of the Hepatitis B DNA virus have been shown to be targets of PARP13. How viral RNA is detected by PARP13 is currently not known, and although binding to PARP13 is a requirement for viral RNA degradation, no motifs or structural features common to the known targets have been identified.

PARP13 binds to multiple components of the cellular 3′-5′ mRNA decay machinery including polyA-specific ribonuclease (PARN), and subunits of the exosome exonuclease complex, RRP46/EXOSC5 and RRP42/EXOSC7. Recruitment of these decay factors results in the 3′-5′ cleavage of viral RNAs bound to PARP13. Although 5′-3′ RNA decay has also been shown to play a role in PARP13-mediated viral degradation, proteins involved in this process including the decapping factors DCP1 and DCP2 and the 5′-3′ exonuclease XRN1, do not bind to PARP13 directly and are instead recruited by other PARP13 binding partners such as DDX17.

Whether or not PARP13 binds to and modulates cellular RNAs either in the absence or presence of viral infection is unknown. However several indications point towards a role for PARP13 in cellular RNA regulation: 1) both PARP13 isoforms are expressed at high levels in cells, however only PARP13.2 expression is upregulated during viral infection suggesting that PARP13.1 has functions unrelated to the antiviral response; 2) even in the absence of viral infection, PARP13 localizes to RNA rich stress granules—non-membranous ribonucleoprotein structures that form during cellular stress in order to sequester mRNAs and inhibit their translation; 3) PARP13 regulates the miRNA pathway by targeting Argonaute proteins for ADP-ribosylation and this regulation occurs both in the absence and in the presence of viral infection. This suggests that PARP13 targeting of RNA to cellular decay pathways could also occur in the absence of viral infection, and that PARP13 could therefore function as a general regulator of cellular mRNA.

Deregulation of gene expression is a hallmark of many diseases, one of the most devastating of which is cancer. Cellular mRNA stability plays a key role in development and propagation of some tumors, autoimmunity, and many inflammatory disorders. The transcripts of many oncoproteins, cytokines, cyclins and G protein-coupled receptors have very labile mRNAs, whose levels are induced for short times in acute response to external signals. Abnormal stability of transcripts, and therefore persistently high levels of transcripts and proteins, often leads to disease conditions. RNA processing is an important component of regulated gene expression in eukaryotic cells. The rates of transcription, pre-mRNA splicing, mRNA transport, translation and degradation determine the steady-state amount of mRNA, and as a result the amount of protein, that will be available to the cell. In many cases, each of these processes involves highly specific protein-RNA interactions. The interactions involve specific recognition of sequences and structural elements in mRNA molecules by the proteins. Accordingly, there is a need to discover new methods for modulating protein-RNA interactions to regulate gene expression for the treatment of disorders (e.g., cancer, immune disorders, viral disorders, and autoimmune disorders).

### SUMMARY OF THE INVENTION

The present invention features a method of treating or decreasing the likelihood of developing a disorder associated with immune misregulation, a viral disorder, or a virus-associated disorder in a subject, the method comprising administering to the subject a therapeutically effective amount of a composition comprising an activator of a CCCH zinc finger-containing PARP, thereby treating or decreasing the likelihood of developing the disorder associated with immune misregulation, the viral disorder, or the virus-associated disorder in the subject.

The present invention also features a method of modulating a CCCH zinc finger-containing PARP-RNA interaction, the method comprising contacting a CCCH zinc finger-containing PARP protein or a a CCCH zinc finger-containing PARP fusion protein with a CCCH zinc finger-containing PARP activator, wherein the contacting results in the modulation of the CCCH zinc finger-containing PARP-RNA interaction.

In one embodiment, the disorder associated with immune misregulation is an autoimmune disorder, wherein the autoimmune disorder is selected from the group consisting of systemic lupus erythematosus (SLE), CREST syndrome (calcinosis, Raynaud's syndrome, esophageal dysmotility, sclerodactyl, and telangiectasia), opsoclonus, inflammatory myopathy, systemic scleroderma, primary biliary cirrhosis, celiac disease, dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, LambertEaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and Neuromyelitis optica.

In a second embodiment, the viral disorder or the virus-associated disorder is selected from the group consisting of infections due to the herpes family of viruses such as EBV, CMV, HSV I, HSV II, VZV and Kaposi's-associated human herpes virus (type 8), human T cell or B cell leukemia and lymphoma viruses, adenovirus infections, hepatitis virus infections, pox virus infections, papilloma virus infections, polyoma virus infections, infections due to retroviruses such as the HTLV and HIV viruses, Burkitt's lymphoma, and EBV-induced malignancies.

In one aspect of the invention, the composition comprising the activator of a CCCH zinc finger-containing PARP is formulated for improved cell permeability.

In another aspect of the invention, the activator of a CCCH zinc finger-containing PARP is iso-ADP-ribose, poly-ADP-ribose, or a derivative thereof.

In yet another aspect of the invention, the composition is administered in combination with a second agent, where the second agent is an immunosuppressant selected from the group consisting of: a calcineurin inhibitor, cyclosporine G tacrolimus, a mTor inhibitor, temsirolimus, zotarolimus, everolimus, fingolimod, myriocin, alemtuzumab, rituximab, an anti-CD4 monoclonal antibody, an anti-LFA1 monoclonal antibody, an anti-LFA3 monoclonal antibody, an anti-CD45 antibody, an anti-CD19 antibody, monabatacept, belatacept, azathioprine, lymphocyte immune globulin and anti-thymocyte globulin [equine], mycophenolate mofetil, mycophenolate sodium, daclizumab, basiliximab, cyclophosphamide, prednisone, prednisolone, leflunomide, FK778, FK779, 15-deoxyspergualin, busulfan, fludarabine, methotrexate, 6-mercaptopurine, 15-deoxyspergualin, LF15-0195, bredinin, brequinar, and muromonab-CD3 or wherein the second agent is an antiviral agent selected from the group consisting of an interferon, an amino acid analog, a nucleoside analog; an integrase inhibitor, a protease inhibitor, a polymerase inhibitor, and a transcriotase inhibitor.

In another embodiment of the invention, administering the composition results in a modulation of an interaction between a CCCH zinc finger-containing PARP and an RNA.

In particular embodiments the modulation is an increase in binding of the CCCH zinc finger-containing PARP to the RNA. In one aspect, the increase in binding results in a decrease in expression or activity of a gene encoded by the RNA. Preferably, the gene encoded by the RNA is selected from any one of the genes listed in Tables 2, 4, or 6, most preferably, any one of the genes listed in Table 4. In another aspect, the increase in binding results in an increase in expression or activity of a gene encoded by the RNA. Preferably, the gene encoded by the RNA is selected from any one of the genes listed in Table 1, 3, or 5, most preferably, any one of the genes listed in Table 3.

In another embodiment, the CCCH zinc finger-containing PARP is a multiple tandem CCCH zinc finger-containing PARP, wherein the multiple tandem CCCH zinc finger-containing PARP is a PARP12 or a PARP13. Preferably, the PARP13 is PARP13.1. In a preferred embodiment, an increase in binding of PARP13 to a RNA results in an increase in expression or activity of a gene encoded by the RNA, wherein the gene encoded by the RNA is TRAIL4.

The present invention further features a method of treating a TRAIL-resistant disorder in a subject, the method comprising administering to the subject a composition comprising an activator of a CCCH zinc finger-containing PARP in a therapeutically effective amount to treat the TRAIL-resistant disorder in the subject.

In one embodiment, the TRAIL-resistant disorder is a cancer selected from the group consisting of colon adenocarcinoma, esophagas adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, lymphoma, and non-Hodgkin's lymphoma.

In one aspect, the composition is administered in combination with TRAIL therapy. In another aspect, administration of the composition to the subject in need thereof sensitizes the subject to the TRAIL therapy. In yet another aspect, administration of the composition increases the binding of PARP13 to TRAILR4 mRNA, wherein the increase binding results in suppression of TRAILR4 expression or activity.

Finally, the present invention features a method of identifying a candidate compound useful for treating an autoimmune disorder, viral or virus-associated disorder, or a TRAIL-resistant disorder in a subject, the method comprising: (a) contacting a PARP13 protein or fragment thereof, with a compound; and (b) measuring the activity of the PARP13, wherein an increase in PARP13 activity in the presence of the compound identifies the compound as a candidate compound for treating the autoimmune disorder, viral or virus-associated disorder, or a TRAIL-resistant disorder.

In one aspect of this invention, an increase in PARP13 activity is an increase in binding of PARP13 to a RNA encoding a gene listed in any one of Tables 1-6. In preferred embodiments, the gene encoded by the RNA is TRAILR4.

In another aspect, the increase in binding of PARP13 to the RNA results in an increase or decrease in expression or activity of the gene encoded by the RNA.

In yet another aspect, the compound is selected from a chemical library, or wherein the compound is an RNA aptamer, or wherein the compound is a small molecule

### DEFINITIONS

By “expression” is meant the detection of a gene or polypeptide by methods known in the art. For example, DNA expression is often detected by Southern blotting or polymerase chain reaction (PCR), and RNA expression is often detected by Northern blotting, RT-PCR, gene array technology, or RNAse protection assays. Methods to measure protein expression level generally include, but are not limited to, Western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), and flow cytometry, as well as assays based on a property of the protein including, but not limited to, enzymatic activity or interaction with other protein partners.

By the term “cell lysate” is meant the contents of the cell once the plasma membrane has been disrupted or permeabilized. Cell lysate also includes the contents of the intracellular organelles (e.g., endoplasmic reticulum, nucleus, mitochondria, chloroplasts, Golgi apparatus, and lysosome) upon disruption of their respective membranes. Cell lysate contains an unpurified mixture of proteins, small molecule metabolites, and nucleic acids (e.g., DNA and RNA). Cell lysate may be prepared from any type of cell, e.g., a mammalian cell (e.g. human, mouse, rat, and monkey cell), a bacterial cell, fungal cell, and a yeast cell. Cell lysate may be obtained by any methods known in the art including physical disruption (e.g., sonication, homogenization, or freeze/thaw procedures) or chemical disruption (e.g., treatment with a detergent (e.g., Triton-X-100 and NP-40)). Cell lysate may be prepared from a cell expressing the nucleic acid that the PARP13 protein and/or the PARP13 fusion protein. Cell lysate may also be prepared from a cell arrested in a specific stage of the cell cycle (e.g., mitosis or S-phase) or may be prepared from asynchronous cells.

By “labeled nicotinamide adenine dinucleotide” or “labeled NAD⁺” is meant a molecule of nicotinamide adenine dinucleotide (NAD⁺) that is covalently labeled with a fluorescent molecule, a colorimetric molecule, or a molecule that is recognized by a specific partner protein (e.g., biotinylation), or labeled with a radioisotope. One example of a labeled NAD⁺ is biotinylated NAD⁺ (e.g., 6-biotin-14-NAD). Examples of radiolabeled NAD⁺ include, but are not limited to, ¹⁴C-adenine-NAD⁺, ³²P-NAD⁺, and ³H-NAD⁺. Additional examples of labeled NAD⁺ are known in the art.

By “modulating a CCCH zinc finger-containing PARP-RNA interaction” is meant increasing or decreasing the specific or nonspecific binding of a CCCH zinc finger-containing PARP (e.g., PARP7 (SEQ ID NO:4), PARP12 (SEQ ID NO:3), or PARP13 (e.g., PARP13.1 (SEQ ID NO:1) or PARP13.2 (SEQ ID NO:2))) to an RNA transcript (e.g., a gene listed in any one of Tables 1-6). For example, modulation of the PARP13-RNA interaction can further result in a decrease or increase expression in the RNA transcript (e.g., a gene listed in any one of Tables 1-6).

By “PAR” or “poly-ADP ribose” is meant a chain of two or more ADP-ribose molecules. The two or more molecules of ADP-ribose making up PAR may occur in a single linear chain or as a branched chain with one or more branches (e.g., at least 1, 2, 3, 4, 5, 6, 7, 8, 9, or 10 branches). Poly-ADP ribose may be attached to a specific substrate (e.g., protein, lipid, DNA, RNA, or small molecule) by the activity of one or more PARP proteins or PARP fusion proteins (e.g., one or more (e.g., 1, 2, 3, 4, 5, 6, 7, 8, 9, 10, 11, 12, 13, 14, 15, 16, 17, 18, 19, 20, 21, or 22) of PARP1, PARP2, PARP3, PARP3.2, PARP3.3, PARP4, PARP5A, PARP5B, PARP6, PARP7, PARP8, PARP9, PARP10, PARP11, PARP12, PARP13.1, PARP13.2, PARP14, PARP15.1, PARP15.2, and PARP16, or one or more of their respective fusion proteins). Attachment of poly-ADP-ribose to a substrate protein may affect the biological activity of the substrate protein, localization of the protein, or the identity and number of proteins that bind to the target substrate (e.g., protein). PARP proteins may also be modified by the covalent attachment of poly-ADP-ribose. The addition of poly-ADP ribose to a PARP protein may occur by “auto-modification” or “auto-modulation” (i.e., a specific PARP catalyzes the attachment of poly-ADP ribose to itself) or may occur by the activity of one or more (e.g., 1, 2, 3, 4, 5, 6, 7, 8, 9, or 10) other PARP proteins.

By “pharmaceutical composition” is meant a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

By “poly-ADP ribose polymerase 13 nucleic acid” or “PARP13 nucleic acid” is meant any nucleic acid containing a sequence that has at least 80% sequence identity (e.g., at least 81%, 82%, 83%, 84%, 85%, 86%, 87%, 88%, 89%, 90%, 95%, 96%, 97%, 98%, 99%, or even 100% sequence identity) to PARP13.1 (SEQ ID NO:1) or PARP13.2 (SEQ ID NO:2). A PARP13 nucleic acid may encode a protein having additional activities to those described above (e.g., mediates increased stress granule formation, role in progression through mitosis or cytokinesis, and modulation (e.g., increase or decrease) of RNAi function).

By “a CCCH zinc finger-containing PARP” is meant a poly-ADP ribose polymerase protein which contains a CCCH zinc finger domain. A CCCH zinc finger-containing PARP may include, but is not limited to, PARP7, PARP12, PARP13.1, or PARP13.2.

By “a multiple tandem CCCH zinc finger-containing PARP” is meant a poly-ADP ribose polymerase protein which contains more than one (e.g., 2, 3, 4, 5, 6, 7, 8, 9, or 10) CCCH zinc finger domains, such as PARP12 (SEQ ID NO:3), PARP13.1, or PARP13.2.

By “poly-ADP ribose polymerase protein 7” or “PARP7 protein” is meant polypeptide containing a sequence having at least 80% identity (e.g., at least 85%, 90%, 95%, 96%, 97%, 98%, 99%, or even 100% identity) to a protein encoded by a nucleic acid sequence containing the sequence of PARP12 (SEQ ID NO:3). A PARP7 (SEQ ID NO:4) protein may contain one or more (e.g., 1, 2, 3, 4, 5, 6, 7, 8, 9, or 10) post-translational modifications, e.g., phosphorylation and ADP-ribosylation (e.g., at least 1, 2, 3, 4, 5, 6, 7, 8, 9, or 10 ADP-ribose molecules) on one or more amino acid residues. Post-translation modification of a PARP7 protein may occur within a cell (e.g., a transgenic cell described above) or in vitro using purified enzymes. PARP7 protein activity assays may be performed as described herein.

By “poly-ADP ribose polymerase protein 12” or “PARP12 protein” is meant polypeptide containing a sequence having at least 80% identity (e.g., at least 85%, 90%, 95%, 96%, 97%, 98%, 99%, or even 100% identity) to a protein encoded by a nucleic acid sequence containing the sequence of PARP12 (SEQ ID NO: 3). A PARP12 protein may contain one or more (e.g., 1, 2, 3, 4, 5, 6, 7, 8, 9, or 10) post-translational modifications, e.g., phosphorylation and ADP-ribosylation (e.g., at least 1, 2, 3, 4, 5, 6, 7, 8, 9, or 10 ADP-ribose molecules) on one or more amino acid residues. Post-translation modification of a PARP12 protein may occur within a cell (e.g., a transgenic cell described above) or in vitro using purified enzymes. PARP12 protein activity assays may be performed as described herein.

By “poly-ADP ribose polymerase protein 13” or “PARP13 protein” is meant polypeptide containing a sequence having at least 80% identity (e.g., at least 85%, 90%, 95%, 96%, 97%, 98%, 99%, or even 100% identity) to a protein encoded by a nucleic acid sequence containing the sequence of PARP13.1 (SEQ ID NO:1) or PARP13.2 (SEQ ID NO:2). A PARP13 protein may contain one or more (e.g., 1, 2, 3, 4, 5, 6, 7, 8, 9, or 10) post-translational modifications, e.g., phosphorylation and ADP-ribosylation (e.g., at least 1, 2, 3, 4, 5, 6, 7, 8, 9, or 10 ADP-ribose molecules) on one or more amino acid residues. Post-translation modification of a PARP13 protein may occur within a cell (e.g., a transgenic cell described above) or in vitro using purified enzymes. PARP13 protein activity assays may be performed as described herein.

By the term “PARP13 fusion protein” is meant a polypeptide containing a polypeptide tag and a sequence encoded by a nucleic acid containing a sequence having at least 80% sequence identity (e.g., at least 85%, 90%, 95%, 96%, 97%, 98%, 99%, or even 100% identity) to PARP13.1 (SEQ ID NO:1), PARP13.2 (SEQ ID NO: 2). The polypeptide tag of a PARP13 fusion protein may be located at the N- and/or C-terminus of the protein. The polypeptide tag may contain one or more of a fluorescent protein (e.g., a green fluorescence protein), a peptide epitope recognized by specific antibodies, a protein that is bound by a partner binding protein with high affinity (e.g., biotin and streptavidin), a His₆-tag, or one or more (e.g., 1, 2, 3, 4, 5, 6, or 7) protease recognition sequence(s) (e.g., one or more of a TEV protease or Factor Xa protease recognition sequence). PARP13 fusion proteins may be purified using antibodies specific for the polypeptide tag. For example, antibodies specific for the polypeptide tag or proteins that bind specifically to the protein sequence in the polypeptide tag may be bound to a bead (e.g., a magnetic bead) or polymer surface in order to allow for the purification of the PARP13 fusion protein. A PARP13 fusion protein may also be purified and subsequently treated with one or more (e.g., 1, 2, or 3) protease(s) to remove the polypeptide tag from the PARP13 fusion protein. A PARP13 fusion protein preferably has the same cellular localization and biological activity as the wild-type PARP13 protein.

By “a CCCH zinc finger-containing PARP activator” is meant an agent that increases the expression (e.g., mRNA or protein level) and/or the biological activity of a CCCH zinc finger-containing PARP (e.g., PARP7, PARP12, or PARP13 (e.g., PARP13.1 or PARP13.2)). For example, a PARP13 activator may increase the level of PARP13 nucleic acid or PARP13 protein (described above). A PARP13 activator may increase the biological activity of a PARP13 protein including, but not limited to, the ability to attach a poly-ADP-ribose molecule to one or more substrate(s) (e.g., a protein, DNA molecule, RNA molecule, lipid, or small molecule), the ability to interact with a target gene transcript (e.g., any of the target genes listed in Tables 1-6), the ability of a PARP13 protein to bind to one or more of its substrates. For example, a PARP13 activator may be a nucleic acid containing a nucleic acid sequence having at least 80% sequence identity (e.g., at least 85%, 90%, 95%, 96%, 97%, 98%, 99%, or even 100%) to PARP7 (SEQ ID NO:4), PARP12 (SEQ ID NO:3), PARP13.1 (SEQ ID NO:1), or PARP13.2 (SEQ ID NO:2). Specific PARP13 activators may increase the expression and/or the biological activity of PARP13. Examples of PARP13 activators include but are not limited to: iso-ADP-ribose or derivatives thereof, poly-APD-ribose or derivatives thereof, and/or NAD analogues.

By “PARP13 biological activity” is meant the ability of a PARP13 protein or PARP13 fusion protein to localize to stress granules and play a role in the formation or nucleation of stress granules, the ability to inhibit the activity of RNAi in the cell, the ability to interact with cellular RNA, and/or the ability to interact with the exosome. Assays for the measurement of the activity of each specific PARP13 are described herein.

By “pharmaceutically acceptable excipient” is meant any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

By “pharmaceutically acceptable salt” is meant those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharm. Sci. 66(1):1, 1977 and in Pharmaceutical Salts: Properties, Selection, and Use, P.H. Stahl and C.G. Wermuth (eds.), Wiley-VCH, 2008. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palm itate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, and the like.

By the term “purified” is meant purified from other common components normally present within the cell. For example, a purified protein is purified away from the other cellular proteins, nucleic acids, and small metabolites present within the cell. A purified protein is at least 85% pure by weight (e.g., at least 90%, 95%, 96%, 97%, 98%, 99%, or even 100% pure) from other proteins, nucleic acids, or small metabolites present in the cell. A purified nucleic acid is at least 85% free of other contaminating nucleic acid molecules or adjoining sequences found in the cell.

By the term “reduce the likelihood of developing” is meant a reduction (e.g., at least 5%, 10%, 15%, 20%, 25%, 30%, 35%, 40%, 50%, 60%, 70%, 80%, 90%, or 95%) for an individual or a patient population in the chance or rate of developing a specific disease by administering one or more therapeutic agent(s) compared to an individual or patient population not receiving the therapeutic agent. The methods of the invention may also reduce the likelihood of developing one or more (e.g., 1, 2, 3, 4, or 5) symptoms of a stress granule-related disorder or reduce the likelihood of developing one or more (e.g., 1, 2, 3, 4, or 5) symptoms of cancer in a patient population or an individual receiving one or more therapeutic agent(s).

By “resistant to TRAIL-mediated apoptosis” or “TRAIL-resistant disorder” is meant a reduction in effectiveness of a drug (i.e., tumor necrosis factor-related apoptosis-inducing ligand (TRAIL)) in the treatment of a disease or disorder (e.g., cancer). Resistance to TRAIL-mediated apoptosis can occur where the cancerous cells (e.g., malignant tumors) are less sensitive (e.g., 10%, 20%, 30%, 40%, 50%, 60%, 70%, 80%, 90%, 95% less sensitive) to apoptosis induction by TRAIL treatment. Cancerous cells that were originally sensitive to TRAIL-induced apoptosis can become resistant after repeated exposure (acquired resistance) or can be initially resistant to TRAIL-induced apoptosis (primary resistance). Resistance to TRAIL can occur at different points in the signaling pathways of TRAIL-induced apoptosis.

The term “subject” as used herein refers to a vertebrate, preferably a mammal, more preferably a primate, still more preferably a human. Mammals include, without limitation, humans, primates, wild animals, feral animals, farm animals, sports animals, and pets.

As used herein, and as well understood in the art, “treatment” or “treating” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Other features and advantages of the invention will be apparent from the following Detailed Description and the claims.

### DESCRIPTION OF THE DRAWINGS

FIG. 1A is an autoradiogram of PARP13 CLIP reactions performed using wild type (+/+) or PARP13-null (−/−) cells treated with 1 μg/ml or 0.13 μg/ml RNaseA. Triangle indicates molecular weight (MW) of PARP13.1, circle indicates MW of PARP13.2. PARP13 immunoblot (IB) shown below.

FIG. 1B is an autoradiogram of CLIP reactions from SBP-PARP13.1 and PARP13.2 expressed and purified in wild type cells treated with 1 μg/ml RNaseA. PARP13 immunoblots shown below.

FIG. 1C is an autoradiogram of SBP-PARP13.1 and SBP-PARP13.2 CLIP reactions treated with 1 μg/ml or 0.1 μg/ml RNase A. PARP13 immunoblots are shown below.

FIG. 1D are CLIP autoradiograms of endogenous PARP13, SBP-PARP13.1 and SBP-PARP13.2 treated with 1 μg/ml RNase A with or without UV crosslinking (254 nm, 200 mJ). PARP13 immunoblots shown below.

FIG. 1E is a diagram of PARP13 isoforms and mutants.

FIG. 1F are CLIP autoradiograms of SBP-PARP13.1, PARP13.1^{ΔZnF}, PARP13.2 and PARP13.2^{ΔZnF} precipitations treated with 1 μg/ml RNase A. PARP13 immunoblot shown below.

FIG. 1G is an autoradiogram of wild type and mutant PARP13.1 CLIP reactions. PARP13 immunoblot shown below (IB). Numerical values of ³²P signal normalized to protein levels shown above; PARP13.1 RNA binding levels set to 1.

FIG. 1H is a graph of ³²P signals normalized to PARP13.1 protein levels for CLIP analysis shown in FIG. 1G.

FIG. 2A is a set of immunofluorescence images showing localization of PARP13.1, PARP13.2, and RNA binding mutants of PARP13 (PARP13.1^{ΔZnF}, PARP13.1^{VYFHR}, PARP13.2^{ΔZnF}, and PARP13.2^{VYFHR}) in non-stressed (untreated) cells. Scale Bar=20 μm.

FIG. 2B is a set of immunofluorescence images showing localization of PARP13.1, PARP13.2, and RNA binding mutants of PARP13 (PARP13.1^{ΔZnF}, PARP13.1^{VYFHR}, PARP13.2^{ΔZnF}, and PARP13.2^{VYFHR}) in stressed (200 μM Sodium Arsenite) cells. Scale Bar=20 μm.

FIG. 3A is a volcano plot showing transcriptome-wide Log 2-fold changes in mRNA expression in PARP13 knockdowns relative to control knockdowns obtained via Agilent array analysis of total mRNA (n=2 independent experiments). 6 of the top 10 upregulated transcripts are labeled. The remaining mRNA data shown in the figure were obtained using qRT-PCR.

FIG. 3B is a set of immunofluorescence images of cells expressing SBP-PARP13.1, SBP-PARP13.1^{VYFHR}, or SBP-PARP13.2, stained with anti-PARP13 and anti-SBP antibodies, and ER Tracker Red. In merge SBP signal is in green, ER Tracker signal is in red, PARP13 signal is not shown. Scale bar=20 μm.

FIG. 3C is a graph showing relative mRNA levels of CCL5, TRAILR4, OASL, IFIT2, RARRES3 and IFIT3 in PARP13 knockdown relative to control knockdown and in PARP13^{-/-A} HeLa cells relative to wild type cells (n=3 independent experiments, bars represent SD).

FIG. 3D is a set of immunoblots showing PARP13 and pSTAT1 in untransfected cells, or cells transfected with control or PARP13-specific siRNA, untreated or treated with 5 μM Jak1 inhibitor (left) and PARP13 and pSTAT1 in wild-type and PARP13^{-/-} HeLa cells untreated or treated with 100 units/mL IFNγ. GAPDH shown as loading control (right).

FIG. 3E is a graph showing mRNA levels of TRAILR4, CCL5, OASL, IFIT2, RARRES3, and IFIT3 in untransfected PARP13^{-/-} cells and PARP13^{-/-} cells expressing PARP13.1, PARP13.1^{VYFHR}, or GFP (DNA transfection control) relative to HeLa cells (n=3 independent experiments, bars represent SD).

FIG. 4A is a graph showing TRAILR4 mRNA levels (left) and an immunoblot showing protein levels (right) in PARP13 knockdown relative to control. GAPDH shown as loading control. n=3 independent experiments, error bars show SD.

FIG. 4B is a graph showing TRAILR4 mRNA (left) and an immunoblot showing protein levels (right) in PARP13^{-/-} cell lines relative to wild type cells. GAPDH shown as loading control. n=3 independent experiments, error bars show SD.

FIG. 4C is an immunoblot showing TRAILR4 protein levels in wild type, PARP13^{-/-A}, and PARP13^{-/-A} cells expressing PARP13.1 or PARP13.1^{VYFHR} GAPDH shown as a loading control.

FIG. 4D is a set of graphs showing TRAILR4 mRNA levels (Log 2FC) in PARP13 knockdown relative to control knockdown in RPE1, SW480, and HCT116 cells (averages of n=3 parallel reactions (RPE1) or n=3 independent experiments (SW480 and HCT116) shown, error bars show SD) and accompanying immunoblots showing PARP13 knockdown; GAPDH shown as normalizing control.

FIG. 4E is a graph showing TRAILR4 mRNA levels in cells treated with PARP13.1-specific and total PARP13 specific siRNAs relative to control siRNAs (averages of n=3 independent experiments shown, error bars show SD, p>0.05 (n.s.), two-sided t-test comparing the two knockdowns) (left) and immunoblots (right) showing PARP13.1 depletion upon knockdown with PARP13.1 specific siRNA. GAPDH shown as loading control.

FIG. 5A is a graph showing TRAILR4 mature RNA (Exon1/Exon3 primer) and pre-mRNA (Intron6-Exon7, Intron 8/Exon9 primers) levels in PARP13 knockdown relative to control knockdown (mean of n=3 independent experiments, error bars show SD).

FIG. 5B is a graph showing normalized Renilla/Firefly luminescence for psiCHECK2 empty vector, psiCHECK2 expressing Renilla-GAPDH 3′UTR (GAPDH 3′UTR), and psiCHECK2 expressing Renilla-TRAILR4 3′UTR (TRAILR4 3′UTR) expressed in wild type or PARP13^{-/-A} cells (mean of n=3 independent experiments, error bars represent SD, p<0.01 (**), two sided t-test).

FIG. 5C is a diagram of Renilla-TRAILR4 3′UTR construct identifying AU-rich element (ARE), ZAP responsive element (ZRE), and miRNA binding sites for miR-133; triangle shading indicates relative length of motif-darker shades correspond to longer motifs and fragments used in 3′UTR destabilization assay. Specific ARE sequences and locations are shown in FIG. 13. Blue fragments exhibited PARP13-dependent destabilization whereas red fragments were not regulated.

FIG. 5D is a graph showing relative PARP13-dependent destabilization for each 3′UTR fragment, represented by fraction increase of normalized Renilla luminescence in PARP13^{-/-A} cells relative to wild type cells, (means of n=3 independent experiments, error bars show SD, asterisks represent significance relative to empty vector, p<0.05 (*), p<0.01(**) and p<0.001 (***), two-sided t-test).

FIG. 5E is a graph showing fold enrichment (Log 2) of TRAILR4 mRNA in input and bound fraction in PARP13.1 CLIP reactions relative to PARP13.1^{VYFHR} reactions (mean of n=3 independent experiments, error bars show SD, p<0.01 (**), two-sided t-test) and accompanying PARP13 immunoblot shows precipitated protein levels.

FIG. 5F is a graph showing relative log 2 levels of TRAILR4 mRNA in input and bound fractions in PARP13.1 CLIP reactions relative to PARP13.1^{ΔZnF} reactions (averages of n=3 independent experiments, bars show SD, p<0.05 (*) relative to PARP13.1^{ΔZnF}, two-sided t-test) and accompanying PARP13 Immunoblot of precipitated protein.

FIG. 5G is an Electrophoretic Mobility Shift Assay (EMSA) of decreasing amounts of PARP13.1 and PARP13.1^{VYFHR} (from 533 nM to 71 nM, in 25% interval decrease) with radiolabeled Fragment E and Fragment 1 (experiment was repeated 3 times with similar results) (left) and Coomassie stain showing equal protein concentration of PARP13.1 and PARP13.1^{VYFHR} (right).

FIG. 6A is a graph showing TRAILR4 mRNA levels in EXOSC5 and XRN1 knockdowns relative to control knockdown (means of n=3 independent experiments, bars show SD, asterisks represent significance relative to control siRNA, p<0.05 (*), p>0.05 (n.s.), two-sided t-test).

FIG. 6B is a graph showing EXOSC5 mRNA levels in EXOSC5 knockdown relative to control knockdown (bars show SD, n=3 independent experiments) (left) and accompanying immunoblot showing XRN1 protein levels in control and XRN1 knockdown (right). GAPDH is shown as loading control.

FIG. 6C is a graph showing relative TRAILR4 mRNA levels in Tet-treated or untreated HEK293 cells expressing Tet-inducible Ago2 shRNA, treated with control or PARP13-specific siRNA (averages of n=3 parallel reactions, error bars show SD) (left) and accompanying immunoblots of PARP13, Ago2, and GAPDH (loading control) (right).

FIG. 6D is a set of bar graphs showing normalized Renilla luminescence for empty vector and Renilla-TRAILR4 3′UTR, expressed in wild type (left bar) or PARP13^{-/-A} cells (right bar) treated with control siRNA or siRNA specific for EXOSC5 or XRN1. PARP13-dependent destabilization levels, calculated by subtracting normalized Renilla luminescence signal in wild type cells from signal in PARP13^{-/-} cells, is shown at left of the bars. (means of n=3 independent replicates, bars show SD, asterisks represent significance relative to control siRNA destabilization levels, p<0.001(***), p>0.05 (n.s.).

FIG. 6E is a set of graphs showing decay of GAPDH mRNA and TRAILR4 mRNA in wild-type and PARP13^{-/-} cells measured by qRT-PCR of 4-thiouridine incorporated and purified RNA. At each time point GAPDH and TRAILR4 levels were normalized to ACTB levels. Levels at Time 0 were set as 0. (means of n=3 independent experiments, error bars show SD, asterisks represent significance relative to wild type levels for each time point, p<0.05(*), p<0.01(**), two-sided t-test).

FIG. 7A is a graph showing percent survival of untreated and TRAIL treated wild type cells and wild type cells expressing TRAILR4-Flag assayed via Annexin-V/PI FACS (average of n=3 independent experiments, bars represent SEM, p<0.05(*), two-sided t-test).

FIG. 7B is a set of immunoblots of TRAILR1-2 and TRAILR4 proteins in wild-type and PARP13^{-/-A} cells. GAPDH is used as loading control.

FIG. 7C is a set of survival assay graphs showing proliferation of SW480 (left), HCT116 (center), and HeLa (right) cells with or without PARP13 knockdown after treatment with increasing concentrations of TRAIL for 24 h. Results are shown relative to untreated cells (means of n=3 independent experiments, error bars show SEM). Results for double knockdown of TRAILR4 and PARP13 are shown for HeLa cells.

FIG. 7D is a graph showing TRAILR4 mRNA levels after PARP13 and PARP13+TRAILR4 knockdown relative to control knockdown (averages of n=3 independent experiments, error bars show SD).

FIG. 7E is a graph showing Annexin-V/PI apoptosis assays comparing the percent survival of wild type and three independent PARP13^{-/-} cell lines (A, B, C) upon 1 μg/ml TRAIL treatment for 24 h (n=3 independent experiments, bars show SEM, asterisks show significance relative to wild type, p<0.001 (***), two-sided t-test).

FIG. 7F is a graph showing normalized survival of wild type and three PARP13^{-/-} cell lines treated with 1 μg/ml TRAIL relative to untreated (averages of n=3 independent experiments, bars show SEM, asterisks show significance relative to wild type, p<0.05 (*) or p<0.01(**), two-sided t-test).

FIG. 7G is an image of the results of a colony formation assay measured by crystal violet staining of wild type or PARP13^{-/-A} cells treated with or without the indicated amounts of TRAIL for 7 days.

FIG. 7H is a graph showing Annexin-V/PI apoptosis assays comparing the percent survival of wild type, PARP13^{-/-A} or PARP13^{-/-A} cells expressing PARP13.1, PARP13.1^{VYFHR} or PARP13.1^{ΔZnF} upon treatment with or without 1 μg/ml TRAIL for 24 h. Data is shown as % survival (means of n=3 independent experiments, bars show SEM, asterisks show significance relative to wild type, p<0.05 (*), p<0.01(**), p>0.05 (n.s.), two-sided t-test).

FIG. 7I is a graph showing normalized survival of wild type, PARP13^{-/-A} cells and PARP13^{-/-A} cells expressing PARP13.1, PARP13.1^{VYFHR} or PARP13.1^{ΔZnF} treated with 1 μg/ml TRAIL relative to untreated (n=3 independent experiments, bars show SEM, asterisks represent significance relative to wild type, p<0.05 (*), p<0.01(**), p<0.001(***), two-sided t-test).

FIG. 8A is an immunoblot examining caspase-8 cleavage at various time points after 1 μg/ml TRAIL treatment in wild-type and PARP13^{-/-} cells. Arrows indicate full-length (FL) caspase-8 and its cleavage products. GAPDH shown as loading control.

FIG. 8B is a set of immunoblots of Flag-TRAIL pulldown of the TRAIL-receptor complex in wild-type and PARP13^{-/-A} cells blotted for TRAILR1, R2, and caspase-8. Inputs for the reaction are also shown.

FIG. 8C is a model of CCCH zinc finger-containing PARP-dependent TRAILR4 mRNA regulation and its effects on TRAIL mediated apoptosis.

FIG. 9 is a set of PARP13 and GAPDH immunoblots (top) and PARP13 immunofluorescence staining (bottom) of wild-type HeLa cells and three independently isolated PARP13^{-/-} cell lines (PARP13^{-/-A}, PARP13^{-/-B} and PARP13^{-/-C}). Scale bar=50 mm.

FIG. 10 is a set of immunofluorescence images showing PARP13 colocalizes with eIF3 at stress granules. Costaining of exogenously expressed SBP-PARP13.1, PARP13.1^{ΔZnF}, PARP13.1^{VYFHR}, PARP13.2, PARP13.2^{ΔZnF}, or PARP13.2^{VYFHR} (SBP, green) with endogenous PARP13 (red) and the stress granule marker eIF3 (blue) in wild-type cells treated with 200 mM sodium arsenite. Scale bar=20 mm.

FIG. 11 is gene Set Enrichment Analysis (GSEA) plot identifying enrichment of interferon pathway components among upregulated transcripts with p<0.05. NES and FDR are reported.

FIG. 12 is a graph showing PARP13.1 but not PARP13.1^{ΔZnF} rescues TRAILR4 mRNA levels in PARP13^{-/-A} cells. TRAILR4 mRNA levels in PARP13^{-/-} cells and PARP13^{-/-} cells expressing PARP13.1 and PARP13.1^{ΔZnF} relative to wild type cells (averages of n=3 independent experiments, error bars show SD, asterisks show significance relative to PARP13^{-/-}, p<0.01 (**), p>0.05 (n.s.), two sided t-test).

FIG. 13 is an ARESITE-derived schematic of AU-rich elements present in the TRAILR4 3′UTR.

FIG. 14 is a set of RNAFold-derived Minimum Folding Energy (MFE) predictions of secondary structure for full-length TRAILR4 3′UTR and 3′UTR fragments described herein. Arrows point to fragment boundaries in the full-length 3′UTR.

FIG. 15 is a graph showing normalized Renilla/Firefly luminescence for 3′UTR fragment constructs in wild type and PARP13^{-/-A} cells (averages of n=3 independent replicates shown, error bars show SD, asterisks represent significance relative to PARP13^{-/-} for each fragment, p<0.05 (*), p<0.01(**), p<0.001(***), two-sided t-test)

FIG. 16 is a graph showing quantitation of TRAILR1-R4 mRNA levels in the three PARP13^{-/-} cell lines relative to wild-type cells (averages of n=3 independent experiments, error bars show SD).

FIG. 17A is a schematic showing the domain structures of all PARP family members.

FIG. 17B is a diagram showing the detailed view of the CCCH-zinc finger containing PARP subfamily. PARP12, PARP13.1, and PARP13.2 have multiple tandem CCCH-zinc fingers and grouped together as the multiple tandem CCCH-zinc finger containing PARPs.

### DETAILED DESCRIPTION

We have discovered that PARP13 binds to and regulates cellular RNA in the absence of viral infection, and that its depletion results in significant misregulation of the transcriptome with an enrichment in signal peptide containing transcripts and immune response genes. From the list of PARP13-dependent differentially expressed genes described in detail herein, we focused on understanding how PARP13 regulates TRAILR4—a member of a family of transmembrane receptors composed of TRAILR1-4 (Johnstone et al., Nature reviews. Cancer 8:782-298 (2008); Degli-Esposti et al., Immunity 7:813-820 (1997)) that bind to TRAIL, a proapoptotic TNF-family cytokine. Primary cells are TRAIL resistant; however many transformed cells become sensitive to TRAIL induced apoptosis, making it an attractive target for the therapeutic treatment of cancers (Johnstone et al., Nature reviews. Cancer 8:782-798 (2008)). TRAIL binding to TRAILR1 and TRAILR2 triggers the assembly of the Death Inducing Signaling Complex (DISC) (Kischkel et al., Immunity 12:611-620 (2000); Sprick et al., Immunity 12:599-609 (2000)) leading to the recruitment and activation of caspase-8 and induction of the extrinsic apoptotic pathway. In contrast, TRAILR3 and TRAILR4 act as prosurvival decoy receptors that bind TRAIL but cannot assemble functional DISCs and therefore cannot signal apoptosis (Merino et al., Molecular and cellular biology 26:7046-7055 (2006); Marsters et al., Current biology:CB 7:1003-1006 (1997)). The relative expression of each receptor varies in different cancers and tissue types and is thought to be important for the overall cellular response to TRAIL (LeBlanc et al. Cell death and differentiation 10:66-75 (2003). Accordingly, high levels of these decoy receptors can prevent TRAIL induced cells death and likely contribute to acquired TRAIL resistance in cancer cells (Morizot et al., Cell death and differentiation 18:700-711 (2011)).

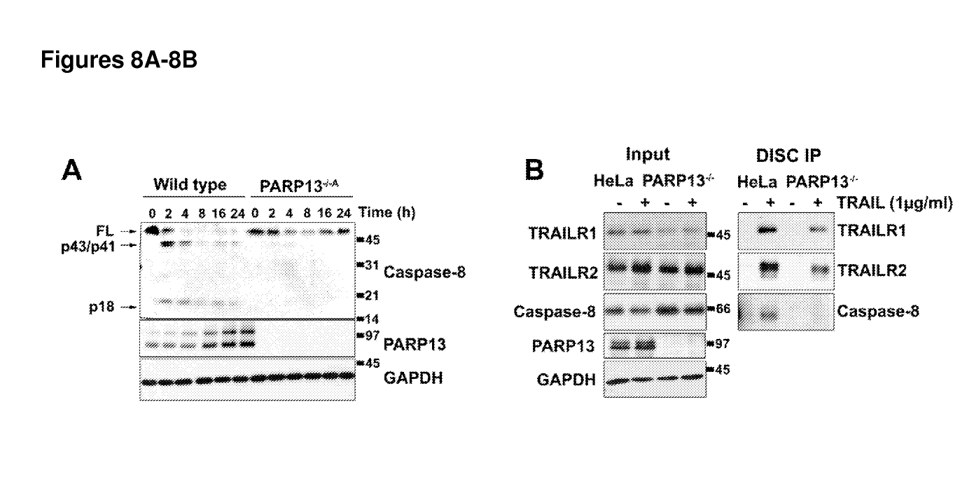

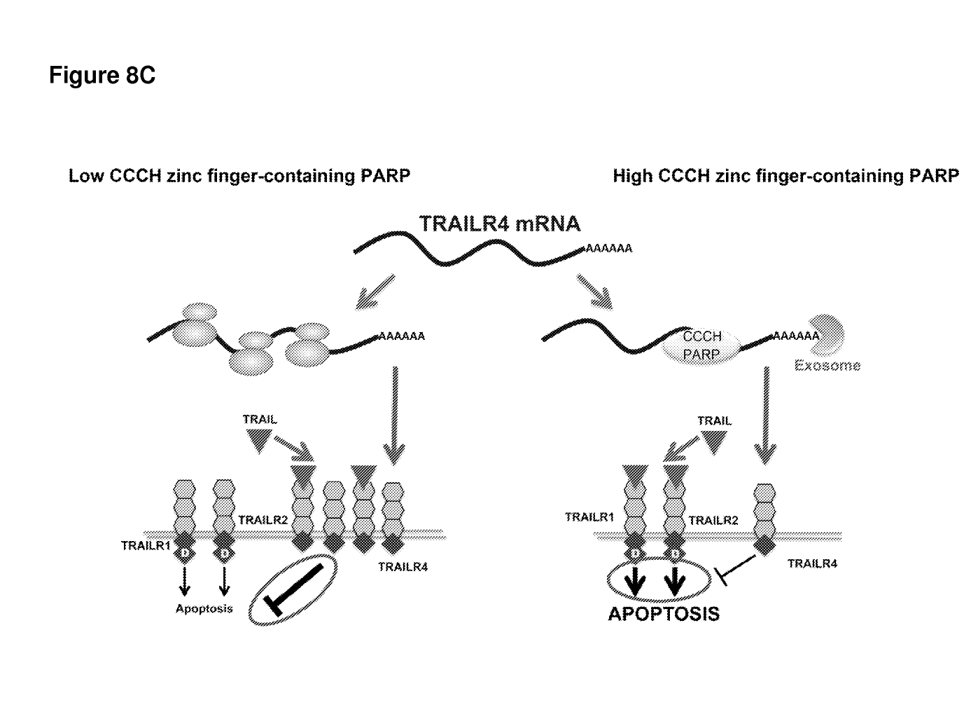

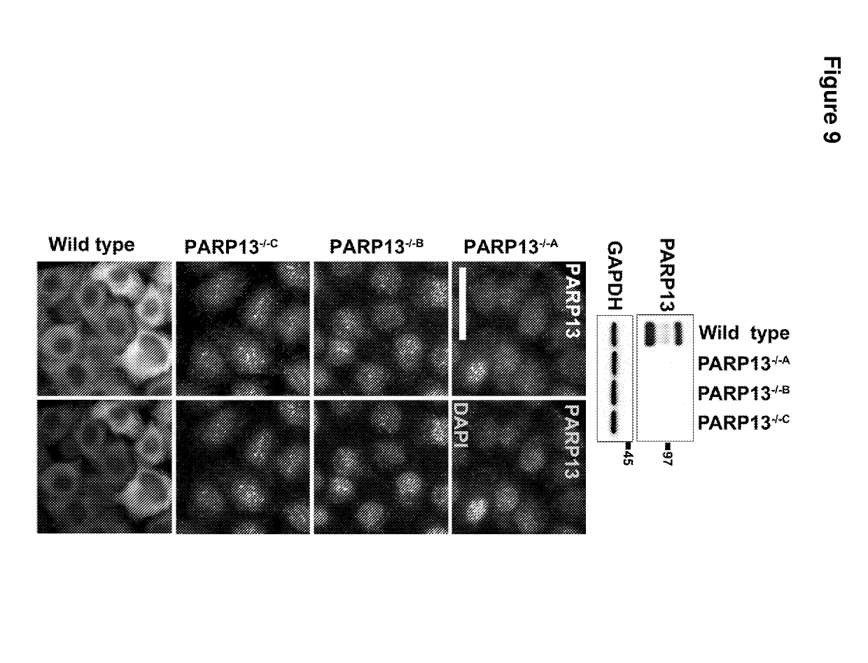

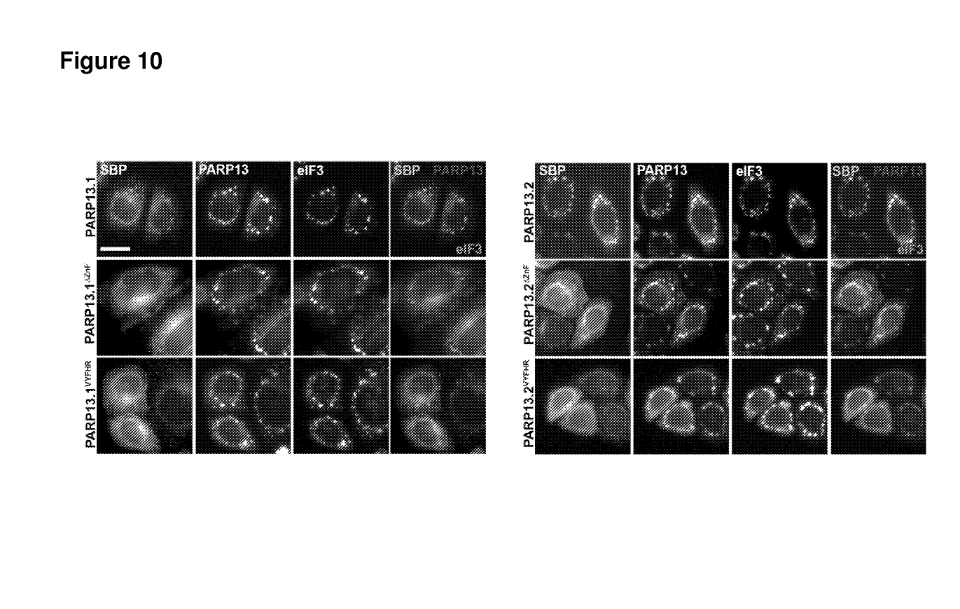

We show that PARP13 destabilizes TRAILR4 mRNA posttranscriptionally but has no effect on the levels of other TRAIL receptors. PARP13 binds to a specific fragment in the 3′ untranslated region (3′UTR) of TRAILR4 mRNA, and leads to its degradation via the RNA exosome complex. Consistent with these data, PARP13 depletion markedly alters TRAILR4 mRNA decay kinetics. By repressing TRAILR4 expression in the cell, PARP13 shifts the balance in the TRAIL signaling pathway towards decreased anti-apoptotic signaling and sensitizes cells to TRAIL-mediated apoptosis (FIG. 8C). These results suggest that PARP13 could have important functions in regulating TRAIL resistance and that modulation of PARP13 may have the potential to overcome TRAILR4 mediated TRAIL resistance. This approach could improve the efficacy of TRAIL therapies currently in clinical trials to target multiple cancers (Stuckey et al., Trends in molecular medicine 19:685-694 (2013)).

Accordingly, the invention provides methods and compositions for treating a disorder associated with immune misregulation (e.g., autoimmune disorders and/or autoinflammatory disorders) and viral disorders by modulating PARP13-RNA interaction. The invention also provides methods and compositions for sensitizing cells to TRAIL-mediated apoptosis for the treatment of TRAIL-resistant cancers. The invention also provides screening methods for the identification of candidate agents that are activators of PARP13 activity and/or expression that may be useful for treating an autoimmune disorder, immune disorder, or viral disorder.

### Screening Assays to Identify One or More CCCH Zinc Finger-Containing PARP Activators

The CCCH zinc finger-containing PARP proteins of the invention (e.g., PARP13 protein) may be used to identify one or more (e.g., 1, 2, 3, 4, 5, 6, 7, 8, 9, 10, 11, 12, 13, 14, 15, 16, 17, 18, 19, or 20 or more) specific PARP13 activators. In the provided assays, the PARP13 protein is contacted with an agent (e.g., a test agent), a labeled NAD⁺ (e.g., a colorimetrically-labeled, fluorescently-labeled, biotinylated-, or radioisotope-labeled NAD⁺), and one or more substrates, and measuring the amount of labeled ADP-ribose covalently attached to the one or more substrates. In one example, the PARP13 protein is incubated with a labeled NAD⁺ substrate and the amount of label associated with the NAD⁺ that is covalently attached to the PARP13 protein is measured (e.g., auto-modulation activity assay). In this example, an agent that is a specific PARP activator mediates an increase (e.g., at least a 5%, 10%, 15%, 20%, 25%, 30%, 35%, 40%, 45%, 50%, 55%, 60%, 65%, 70%, 80%, 90%, 95%, or even 100% increase) in the amount of labeled ADP-ribose covalently attached to the PARP13 protein, wherein the label on the PARP13 protein is the same as the label of the NAD⁺.

The CCCH zinc finger-containing PARP (e.g., PARP12, PARP13.1, or PARP13.2) protein utilized in each assay may be purified, partially purified (e.g., at least 30%, 40%, 50%, 60%, 70%, 80%, 85%, 90%, or 95% pure) or may be present in a cell lysate (e.g., a bacterial cell lysate, a yeast cell lysate, or a mammalian cell lysate), in a biological fluid from a transgenic animal (e.g., milk or serum), or an extracellular medium. The CCCH zinc finger-containing PARP (e.g., PARP12, PARP13.1, or PARP13.2) protein utilized in the assay may be bound to substrate, such as, but not limited to, a solid surface (e.g., a multi-well plate), a resin, or a bead (e.g., a magnetic bead). In additional examples of the assays, the CCCH zinc finger-containing PARP (e.g., PARP12, PARP13.1, or PARP13.2) protein may be bound to a solid surface, resin, or bead (e.g., a magnetic bead) and subsequently treated with one or more protease(s) (e.g., a TEV protease) prior to contacting the CCCH zinc finger-containing PARP (e.g., PARP12, PARP13.1, or PARP13.2) protein with the labeled NAD⁺.

In preferred assays, an activator increases the amount of labeled ADP-ribose covalently attached to a specific CCCH zinc finger-containing PARP (e.g., PARP12, PARP13.1, or PARP13.2) protein, while having no or little (e.g., less than 50%, 40%, 30%, 25%, 20%, 15%, 10%, or 5% change (e.g., increase or decrease)) affect on the amount of labeled ADP-ribose covalently attached to other PARP proteins, is identified as a CCCH zinc finger-containing PARP (e.g., PARP12, PARP13.1, or PARP13.2) activator. For example, the assay desirably identifies an agent that specifically increases the amount of labeled ADP-ribose covalently attached to PARP13.1 proteins, PARP13.2 proteins, PARP12 proteins, and/or fusion proteins.

A variety of different agents may be tested in the above-described assays provided by the invention. For example, a tested agent may be a derived from or present in a crude lysate (e.g., a lysate from a mammalian cell or plant extract) or be derived from a commercially available chemical libraries. Large libraries of natural product or synthetic (or semi-synthetic) extracts or chemical libraries are commercially available and known in the art. The screening methods of the present invention are appropriate and useful for testing agents from a variety of sources for activity as a specific PARP activator. The initial screens may be performed using a diverse library of agents, but the method is suitable for a variety of other compounds and compound libraries. Such compound libraries can also be combinatorial libraries. In addition, compounds from commercial sources can be tested, as well as commercially available analogs of identified inhibitors.

An agent may be a protein, a peptide, a DNA or RNA aptamer (e.g., a RNAi molecule), a lipid, or a small molecule (e.g., a lipid, carbohydrate, a bioinorganic molecule, or an organic molecule).

The invention also provides methods for identifying an agent that specifically binds to the PARP13 protein. These methods require the contacting of the PARP13 protein of the invention with a test agent and determining whether the test agent specifically binds to the PARP13 protein. An agent that specifically binds PARP13 protein (e.g., an agent that specifically binds to PARP13 at its WWE domain) may act as an activator of the expression or activity of the PARP13 protein in a cell. For example, an agent that specifically binds to PARP13 protein may selectively increase the activity or expression of the PARP13 protein in the cell or sample.

The PARP13 protein used in this method may be attached to a solid surface or substrate (e.g., a bead) and/or may be present in purified form or present in a crude cell lysate, biological fluid, or extracellular medium. The methods may optionally include one or more (e.g., 1, 2, 3, 4, or 5) washing steps following contacting the PARP13 protein with the test agent. The test agent may be a small molecule, a lipid, an RNA molecule, a DNA molecule, a protein, or a peptide fragment. The test agent may be purified in form (e.g., at least 70%, 80%, 85%, 90%, 95%, or 99% pure by weight) or may be present in a crude cell lysate. The test agent may also, optionally be labeled (e.g., a colorimetric label, a radionuclide label, labeled with a biotin molecule, or labeled with a fluorophore).

The binding of the test agent to PARP13 protein may be detected by any known method including, BIAcore, competitive binding assays (e.g., a competitive binding assay using one or more of the antibodies provided by the invention), and detection of the agent following its release from the PARP13 protein (e.g., elution of the bound test agent following exposure to high salt or a high or low pH buffer).

In one example of this method, a bead attached to the PARP13 protein and/or fusion protein thereof may be incubated with a crude cell lysate, and the proteins or peptide fragments bound to the PARP13 protein and/or fusion protein thereof may be eluted from the beads by exposure to a high salt buffer, a high detergent buffer, or a high or low pH buffer. The resulting eluted proteins may be electrophoresed onto an SDS-polyacrylamide gel and the specific protein bands cut out from the gel and analyzed using mass spectrometry to identify the specific agent that binds to the PARP13 protein and/or fusion protein thereof.

In another example of the method, a bead attached to the PARP13 protein and/or PARP13 fusion protein is incubated with a purified protein or peptide fragment. In this instance, a protein or peptide fragment bound to the PARP13 protein and/or PARP13 fusion protein may be eluted using a high salt buffer, a high detergent buffer, or a high or low pH buffer. The amount of protein in the eluate may be detected by any method known in the art including UV/vis spectroscopy, mass spectrometry, or any colorimetric protein dye (e.g., a Bradford assay).

In specific screening assays for agents that bind the PARP13 protein and/or the PARP13 fusion protein, the PARP13 protein and/or PARP13 fusion protein may be placed in individual wells of a multi-well plate (e.g., the PARP13 protein and/or PARP13 fusion protein covalently linked to the plate surface) and incubated with the test agent. Following a washing step, the amount of test agent remaining in each well may be determined and the ability of the test agent to bind the PARP13 protein and/or PARP13 fusion protein determined.

In general, candidate agents/compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

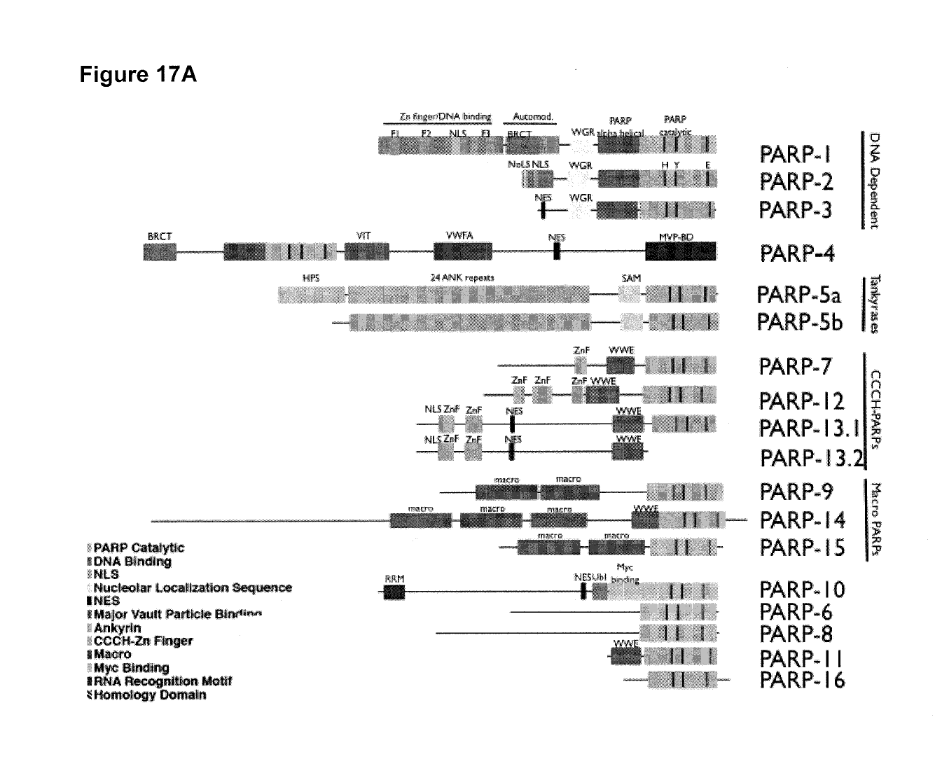

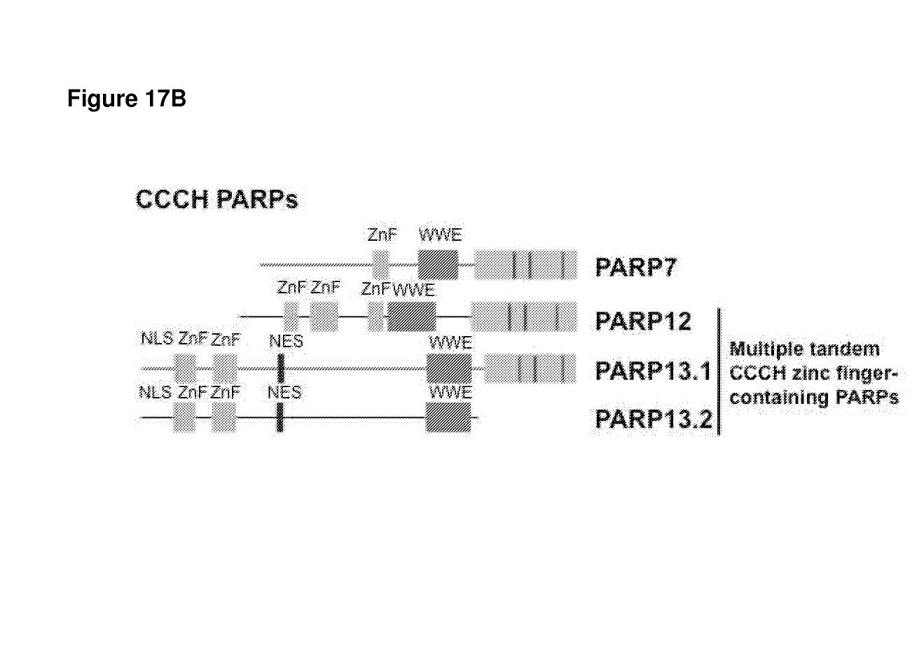

Additionally, it is important to note that PARP13 is just one member of the CCCH zinc finger-containing PARP subfamily identified based on the presence of CCCH RNA binding domains. PARP12 and PARP7 are the other members of the CCCH zinc finger-containing PARP subfamily (see FIGS. 17A and 17B). Both PARP12 and PARP13 function in the antiviral response and localize to membraneous organelles (PARP13 to the ER and PARP12 to the Golgi). PARP12 and PARP13 (i.e., PARP13.1 and PARP13.2) exhibit similar domain structures including the presence of multiple tandem CCCH zinc fingers (see FIG. 17B). Therefore, it is specifically contemplated that PARP12 may regulate cellular RNA in a manner similar to PARP13 and it is within the scope of the invention to identify activators of PARP12.

### Poly-ADP-Ribose and NAD Analogues

The interaction of CCCH zinc finger-containing PARP (e.g., PARP12, PARP13.1, or PARP13.2) with ADP-ribose modifies the ability of the PARP to bind mRNA. For example, it has been shown that PARP13 can both be directly modified by poly-ADP-ribose (Leung et al. RNA Biology 9:542-548 (2012))) and bind to the modifications. These interactions with ADP-ribose change the binding of PARP13 to RNA and affect its ability to regulate its target RNAs. Therefore, targeting the interaction between a CCCH zinc finger-containing PARP and ADP-ribose using an ADP-ribose or NAD analogue is a therapeutic strategy that can be used in known CCCH zinc finger-containing PARP-dependent pathways. The WWE domain of CCCH zinc finger-containing PARP recognizes poly-ADP-ribose (PAR) by interacting with iso-ADP-ribose (iso-ADPR), the smallest internal poly-ADP-ribose structural unit containing the characteristic riboseribose glycosidic bond formed during poly(ADP-ribosyl)ation.

It is within the scope of the invention to use iso-ADP-ribose or derivatives thereof, poly-ADP-ribose or derivatives thereof, and/or NAD analogues as activators of CCCH zinc finger-containing PARP in order to modulate CCCH zinc finger-containing PARP interaction with RNA. The iso-ADP-ribose, poly-ADP-ribose, or derivative thereof, may be unmodified (e.g., unmodified and in a liposome formulation) or modified/derivatized, such that the compound is in a cell-permeable form. Methods of synthesizing iso-ADP-ribose are known in the art, for example, poly-ADP-ribose can be treated with poly-ADP-ribose glycohydrolase to form iso-ADP-ribose and see for example Carter-O'Connel et al., J. Am. Chem. Soc. 136:5201-5204 (2014) for methods of synthesizing poly-ADP-ribose derivatives. Methods of synthesizing NAD analogs are known in the art (for example, see, Pankiewicz et al., Journal of Medicinal Chemistry 36:1855-1859 (1993); Goulioukina et al., Helvetica Chimica Acta 90:1266-1278 (2007)) and analogues are commercially available (see, for example, Jena Bioscience Catalog No. NU-514, NU-515, NU-516, NU-517, NU-518, NU-519, NU-520, NU-521, NU-522, NU-523, and NU-524). Preferably, these small molecule analogues are provided in cell permeable form (e.g., formulated in lipid-based drug delivery systems (Kalepu et al., Acta Pharmaceutica Sinica B 3:361-372 (2013)), bile salts, nano emulsions, cyclodextrin inclusion complex, spray freeze dying, chitosan derivatives, saponins, straight chain fatty acids, self-micro-emulsifying drug delivery systems (SMEDDS), and/or self-double emulsifying drug delivery systems (SDEDDS) (Shaikh M S I et al., Journal of Applied Pharmaceutical Science 2:34-39 (2012)).

### Target Genes

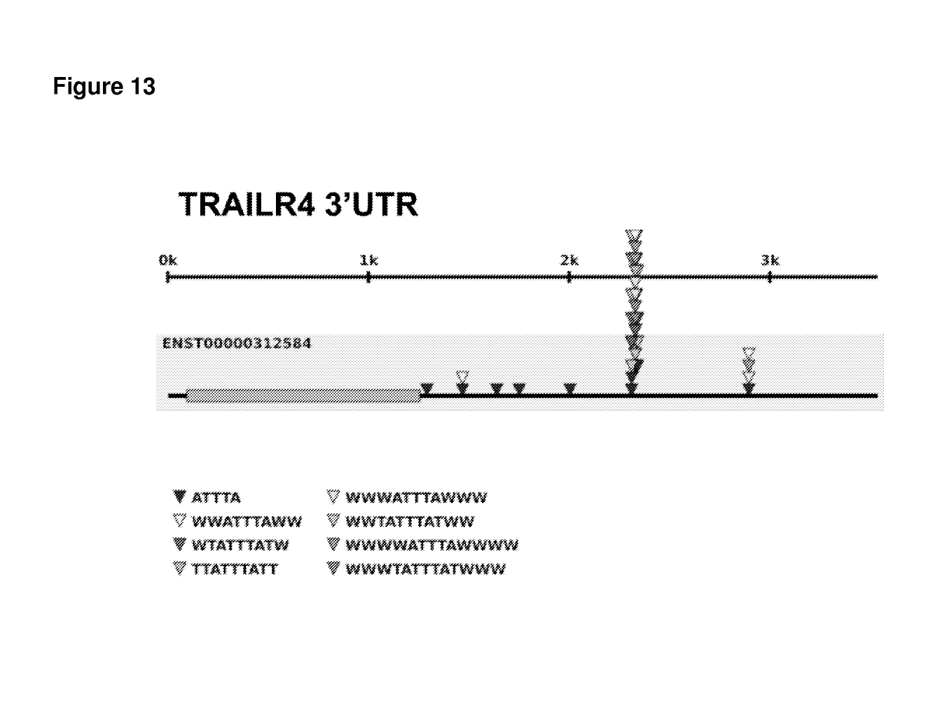

It is an object of the invention to understand the function of CCCH zinc finger-containing PARPs, and, in particular, multiple tandem CCCH zinc finger-containing PARPs, in the regulation of cellular mRNA but addressing the following questions: (1) what are the direct targets of regulation, (2) how is target specificity determined, and (3) does the regulation of cellular targets change upon viral infection. Many of the transcripts misregulated upon knockdown of the CCCH zinc finger-containing PARP, PARP13, identified herein may be indirect targets. To better understand the biology of CCCH zinc finger-containing PARPs, such as PARP13, identifying additional direct targets is critical. Without wishing to be bound by theory, the target recognition of cellular m RNA by CCCH zinc finger-containing PARPs, such as PARP13, is more likely to be mediated by structural features rather than linear sequence motifs. Interestingly, the expanded AU-rich element in the TRAILR4 3′UTR is predicted to form a hairpin with high probability (FIG. 13), suggesting that it may represent a structure recognized by PARP13 (Lorenz et al., Algorithms for molecular biology:AMB 6:26 (2011)).

The highly significant enrichment of signal peptide containing transcripts upon PARP13 depletion (corrected p-value<0.0001) strongly suggests that PARP13 has a specific function in the regulation of transmembrane proteins, or those that are destined to be secreted. This enrichment might be related to PARP13.1 localization at the ER. Indeed, PARP13.1 has been shown to be farnesylated, and this modification targets PARP13.1 to membranes and is required for its antiviral activity (Charron et al., Proceedings of the national Academy of Sciences of the United States of America 110:11085-11090 (2013). It is therefore possible that similar targeting of PARP13.1 to membranes might also regulate its function in destabilizing cellular transcripts, including those at the ER.

The transcriptome was analyzed in the absence of PARP13 to see which cellular RNA transcripts were regulated by PARP13. Depletion of PARP13 resulted in significant misregulation of the transcriptome with 1841 out of a total of 36,338 transcripts analyzed showing >0.5 Log 2 fold change (Log 2FC) relative to control knockdowns (1065 upregulated and 776 downregulated transcripts). Of these, 85 transcripts exhibited Log 2FC>1 relative to control siRNAs (66 upregulated and 19 downregulated). Genes identified as being downregulated or upregulated upon PARP13 depletion by various cutoff p-values and log fold change of expression are further detailed in Tables 1-6.

TABLE 1
Downregulated Genes in PARP13 siRNA cells (Log2FC > 0.5)

| | | | | |
|---|---|---|---|---|
| XLOC_003006 | GDF1 | PTPRH | XLOC_001002 | XLOC_000955 |
| USP7 | NOX3 | IQCG | AB051446 | SNORD114-11 |
| ENST00000422961 | ENST00000390602 | XLOC_004457 | A_33_P3265254 | FMO2 |
| DDX4 | XLOC_010929 | SNORD83A | OR52B2 | TMPRSS12 |
| XLOC_I2_005693 | ZNF83 | XLOC_004836 | XLOC_000780 | NGEF |
| PCP2 | ARMCX3-AS1 | A_33_P3253179 | LOC100130000 | PP12719 |
| XLOC_003578 | JUNB | SPON1 | CORO2A | FLJ46361 |
| LOC100506075 | LOC220729 | UNC5B | C20orf197 | NR0B2 |
| ZNF488 | XLOC_010927 | XLOC_010026 | PAQR6 | SNORD80 |
| ARVCF | VWA5A | XLOC_004841 | LOC100128590 | CARD9 |
| SNORD72 | PALM | MARS2 | XM_001719321 | CNPPD1 |
| TCTE3 | LOC100130453 | SNORD28 | HIST1H1B | SNORD5 |
| HOXA2 | C16orf52 | A_33_P3234442 | ATP6V1G2 | MMP19 |
| P2RX2 | A_33_P3379215 | C10orf113 | FBXO18 | LOC389765 |
| CMBL | XLOC_012440 | THC2772510 | ENST00000421045 | XLOC_003473 |
| LOC283738 | GTF3C1 | SYNPO2 | NBEAL2 | TAS1R1 |
| XLOC_012769 | SFT2D1 | XLOC_001659 | DEFB108B | CCNA1 |
| SNORD100 | AGPHD1 | PRAMEF20 | MYOT | CDO1 |
| CACNG1 | PODN | RTN4RL2 | LOC100506272 | TMEM105 |
| A_33_P3359120 | B3GNT8 | XLOC_I2_001331 | XLOC_005141 | CHCHD7 |
| TMSB4XP1 | MVD | AK4 | PCK2 | LOC100653304 |
| LILRA1 | XLOC_005264 | XLOC_002419 | SNORD114-31 | XLOC_007091 |
| LOC100290566 | XLOC_I2_011983 | SEC24A | ASXL3 | CRABP2 |
| GRAMD4 | A_24_P383330 | XLOC_I2_015821 | HNRNPA2B1 | SLC9A10 |
| NPS | ROGDI | LOC100303749 | LOC100507118 | MICALL1 |
| XLOC_006904 | CDYL2 | LPHN3 | PSAP | XLOC_004679 |
| A_33_P3243600 | MFSD8 | FAM194B | PRKRIR | SNORD114-26 |
| HDAC2 | LOC100131496 | ELL2 | LOC100132815 | LATS1 |
| NR2C1 | KLF9 | XLOC_006289 | ENST00000369586 | XLOC_I2_011656 |
| MC4R | A_33_P3286929 | XLOC_008907 | HIST1H3E | GTF2H4 |
| METTL21D | KPNA5 | RHOT2 | RASL10A | CFLAR-AS1 |
| STK36 | C3orf58 | PAGE2B | YAP1 | SURF4 |
| SDSL | CDH16 | C5orf13 | PDK3 | ENST00000355513 |
| PTPN5 | SLC9B2 | ENST00000390303 | TSPAN17 | UCN2 |
| WRN | COL4A6 | CROCCP2 | XLOC_008942 | CD19 |
| XLOC_001107 | TXNL4B | C21orf63 | OR51B4 | CHMP4B |
| ENST00000522186 | XLOC_006586 | FRA10AC1 | EFCAB2 | XLOC_I2_010963 |
| CERS4 | CHST14 | XLOC_005675 | A_33_P3221318 | PSMD13 |
| XLOC_001825 | ZFP82 | FAM171A2 | GRRP1 | CHID1 |
| FLJ46010 | XLOC_009194 | XLOC_012307 | CSF1R | LOC100128239 |
| ENST00000454167 | LOC643355 | A_33_P3229527 | OTOP2 | SFTA2 |
| HIST1H4G | ANP32D | XLOC_002617 | XLOC_I2_008040 | CCDC106 |
| OR5AN1 | DUSP2 | CD300LF | LHX2 | THC2512536 |
| XLOC_010626 | LOC100505976 | PPP2R1B | XLOC_I2_013883 | EFCAB6 |
| XLOC_012554 | CCR5 | GAB4 | LOC100652917 | RAD17 |
| THC2539563 | SNORD35B | XLOC_I2_005644 | XLOC_011680 | CTRL |
| LRRC8B | XLOC_009458 | LOC100129534 | XLOC_006512 | FAM74A1 |
| XLOC_008272 | C14orf162 | A_33_P3220748 | SEC61A2 | A_32_P148476 |
| C6orf168 | FAM20A | LRRC37A2 | XLOC_I2_015491 | ENST00000301171 |
| TMED10 | TMEM38A | NT5C3L | TTC26 | LOC100506069 |
| TEX15 | C3orf74 | XLOC_009953 | LOC100505702 | CAB39 |
| AURKB | DTWD1 | GREB1 | LOC728558 | ARL5B |
| C15orf27 | PCDHB1 | PTGS1 | BRAP | XLOC_011616 |
| XLOC_001789 | RHOV | FAP | GDAP1L1 | XLOC_I2_005517 |
| XLOC_014066 | SLC12A3 | ZNF806 | BF733045 | ZNF831 |
| XLOC_I2_015034 | ATP5C1 | LOC100128242 | XLOC_001836 | XLOC_010352 |
| XLOC_013046 | OR2AT4 | RNASE11 | ARRDC3 | GPC5 |
| LOC100505639 | MEF2B | GUSB | ANKRD36BP2 | C16orf87 |
| KLHL25 | ACSL6 | SFTPB | A_33_P3307063 | SYNDIG1 |
| L3MBTL1 | XLOC_I2_007827 | CNNM4 | SNX10 | PRUNE |
| OR52N2 | Q9DSJ7 | HEATR3 | FLJ25917 | MFSD4 |
| CHRNA9 | AASDH | ST8SIA5 | THC2573499 | TSPAN7 |
| SLC6A16 | CHRND | LYSMD4 | LOC100506348 | MRPS23 |
| XLOC_003703 | HPCAL4 | ENST00000556145 | A_33_P3228543 | ISL2 |
| TNP1 | LOC100631378 | HIST1H3G | GLRA2 | SNORD42B |
| ASPG | LOC729678 | XLOC_009958 | AGPAT6 | SNORA70C |
| XLOC_I2_005438 | XLOC_I2_015482 | CCDC134 | F8 | TMOD3 |
| XLOC_007154 | PLXNA3 | ENST00000399363 | MFNG | CXCL14 |
| OR8D4 | MAEL | HABP2 | DUSP13 | ST3GAL5 |
| MN1 | CYP4B1 | FAM87A | XLOC_001183 | GLT1D1 |
| C6orf154 | WNT7B | KYNU | NFRKB | C1orf141 |
| CACNB1 | A_33_P3343605 | C17orf59 | C15orf55 | XLOC_009927 |
| XLOC_000578 | A_33_P3279526 | C17orf109 | ENST00000445752 | ENST00000475340 |
| LOC100507284 | C3 | HLA-DRB1 | SNORD109B | LOC727915 |
| HILPDA | MIPOL1 | CD46 | NAMA | GDPD5 |
| MILR1 | XLOC_I2_012319 | DDIT4 | C8orf22 | ATP2A3 |
| PER1 | ALOX12B | FAM66D | SNORD19 | PIK3IP1 |
| MYC | BICD1 | A_33_P3408443 | LOC283663 | XLOC_005566 |
| CORO7 | A_33_P3268318 | LOC653075 | XLOC_003222 | GADD45G |
| PHKG2 | XLOC_I2_014190 | PASK | XLOC_001339 | RARRES1 |
| TMCC2 | ATP1A4 | NNT | GALNT8 | SRPX2 |
| A1CF | GAA | XLOC_007195 | SLC30A3 | FANCB |
| A_33_P3353873 | XLOC_006819 | ERO1L | A_33_P3402993 | A_33_P3255051 |
| XLOC_I2_009639 | EIF2B5 | OR8K1 | NAP1L1 | TNNI3 |
| XLOC_007212 | SLC2A14 | ST6GALNAC6 | C1orf115 | NEIL1 |
| HRK | YARS | B3GNT3 | COL15A1 | CA7 |
| FBXW4 | XLOC_009957 | ENST00000444348 | C12orf35 | TBX3 |
| ACOX2 | A_33_P3407623 | GBX1 | KIAA1704 | A_33_P3391970 |
| ENST00000554451 | A_24_P246777 | CUTC | MAGEA4 | VWA3A |
| ARMCX1 | XLOC_013407 | LOC100499194 | RFNG | ZNF442 |
| ZNF629 | XLOC_013181 | XLOC_014237 | C6orf138 | XLOC_012171 |
| XLOC_007681 | XLOC_005286 | RASSF8 | THC2766186 | POFUT2 |
| PLCZ1 | XLOC_001556 | XLOC_012421 | SLC43A1 | PRKAA1 |
| XLOC_001852 | ADCK4 | CDC14B | A_33_P3419481 | ZNF230 |
| SLC25A40 | RAB3C | ZNF408 | XLOC_010253 | C4orf47 |
| GSTCD | SLC27A6 | LOC157860 | TLX1 | CLEC4F |
| DQX1 | SGCA | DBNDD1 | LINC00473 | XLOC_I2_011669 |
| RNF168 | PITPNM1 | XLOC_006048 | CYTH2 | MB |
| CRYBG3 | ZNF592 | EMB | SNORD125 | ZNF146 |
| C10orf129 | SNORD70 | TRDMT1 | XLOC_011573 | ANKRD35 |
| XLOC_004573 | TXNRD2 | PACSIN3 | XLOC_I2_002171 | SNORA16A |
| PRO0471 | JHDM1D | XLOC_013439 | VGLL1 | TERT |
| PTGS2 | SH3GL1P3 | ELMO3 | GLMN | CDKL3 |
| INHBA | ELOVL3 | ADSSL1 | XLOC_008053 | GIPC3 |
| FA2H | PCOLCE | TP53TG5 | OSBPL8 | CASP8AP2 |
| XLOC_011017 | XLOC_I2_006609 | XLOC_006321 | C10orf11 | XLOC_I2_014797 |
| A_33_P3311001 | ENST00000370702 | LOC100505787 | LOC100505908 | LRRC36 |
| OSCP1 | ATP13A5 | A2M | SNHG4 | TRAP1 |
| SERPINA12 | XLOC_010184 | C12orf5 | LPIN3 | XLOC_014143 |
| OR4K14 | TTC7B | XLOC_013004 | XLOC_I2_011704 | GBA2 |
| TSPYL1 | PTPRZ1 | SAMD8 | A_33_P3402838 | A_33_P3390823 |
| XLOC_004216 | MYOM2 | FOLH1B | THC2708064 | EPHA4 |
| SDR16C6P | PPIL6 | XLOC_I2_004595 | GRAMD1A | KIAA1841 |
| CAMP | C20orf106 | XLOC_I2_001960 | HEY1 | IST1 |
| XLOC_006468 | ZNF774 | ENST00000383418 | SNTB2 | CCL19 |
| TRPV2 | WDR44 | ZP3 | XLOC_011369 | ANGPTL4 |
| SF3B1 | PPP1R1A | RCN1 | INPP5J | GATA2 |
| MOCOS | XLOC_009942 | MAN2A1 | RIMS3 | ALPL |
| XLOC_014071 | CHRD | EN1 | INSIG2 | SNORD32A |
| IQUB | SNORD114-10 | HIST1H2AB | PDIA6 | XLOC_007277 |
| LRP1 | XLOC_003688 | XLOC_004206 | KCNK18 | EFCAB3 |
| POU6F1 | BU963192 | ZC3HAV1 | LOC283454 | TSHZ2 |
| RCOR2 | MAPK14 | AQP9 | CXorf64 | IL17RE |
| BSCL2 | PTX3 | XLOC_004277 | PRDX3 | SLC2A3 |
| CES3 | FBXO25 | ARAF | PPYR1 | NP511204 |
| MEX3A | CERS1 | CLTC | PTP4A3 | FAM182B |
| ARNT2 | PLIN2 | TRPC4 | DPP10 | LOC100127946 |
| ENST00000444694 | QDPR | S1PR3 | RHOU | BIK |
| RAB40AL | ANKZF1 | NTN3 | KCNMB4 | THC2746051 |
| XLOC_005166 | ESPNL | KLHL1 | SERPINA11 | SHOX2 |
| XLOC_008899 | OGDHL | EGR1 | GAL3ST1 | ACBD4 |
| LOC440356 | CRLF1 | A_33_P3419735 | ENDOV | RPL23AP32 |
| FTSJ2 | ATG4C | CLDN15 | THAP5 | XLOC_I2_000092 |
| MAST1 | HMGB4 | ZSCAN21 | COMMD7 | XLOC_001849 |
| USH1C | AOC3 | MAN2B1 | XLOC_010191 | XLOC_006388 |
| LRRC42 | A_33_P3371260 | ZNF57 | XLOC_005252 | ENST00000509713 |
| ETV4 | RGS16 | A_33_P3397298 | WNT6 | PTBP2 |
| C14orf45 | RGPD6 | SLC5A1 | AP1S3 | FLJ31485 |
| AHCY | DACT2 | XLOC_012398 | XLOC_010366 | PRDM14 |
| SLC5A10 | LOC100286969 | IPPK | MLYCD | LOC79015 |
| SLC35A3 | SLC25A41 | LOC100507226 | XLOC_I2_005933 | CTSL2 |
| XLOC_000647 | ZBED2 | XLOC_I2_012847 | IGFBP3 | EFNA3 |
| KRAS | KRBA1 | XLOC_005289 | HIST1H3D | XLOC_011103 |
| XLOC_003963 | LOC100506662 | TNFRSF25 | C12orf75 | ENST00000431767 |
| GPR124 | ZNF837 | XLOC_004293 | C17orf76-AS1 | GPR153 |
| BNIP3 | NELF | THC2516708 | A_33_P3281716 | IFT46 |
| DMBX1 | XLOC_001451 | ACTL8 | LOC728543 | MS4A6A |
| NDRG1 | GSTA4 | RNF182 | PFKFB4 | MIR17HG |
| XLOC_006201 | XLOC_I2_010702 | LOC100289607 | SLCO1B1 | XLOC_003385 |
| SCARA5 | LGSN | FGFR4 | SNORD54 | FZD10 |
| XLOC_010357 | F2RL1 | RNF112 | VNN2 | SEMA4B |
| XLOC_003007 | C17orf103 | TNXB | FBLN2 | ENO3 |
| ALDOC | PRSS8 | FAM3B | HYMAI | BC028053 |
| TFF3 | XLOC_I2_000917 | H19 | NDUFA4L2 | NFE2 |
| CFI | NTS | LOC100506810 | CA9 | SPINK4 |
| CALHM3 | | | | |

TABLE 2
Upregulated Genes in PARP13 siRNA cells (Log2FC > 0.5)

| | | | | |
|---|---|---|---|---|
| CCL5 | TNFRSF10D | OASL | GSTP1 | IFIT2 |
| CLDN18 | GPNMB | C14orf64 | RARRES3 | XLOC_007808 |
| XLOC_003310 | DDX58 | IFIT3 | ISG15 | XLOC_014103 |
| PI3 | ELSPBP1 | COL8A1 | CFH | LOC100506923 |
| IF144 | LOC100127961 | HS3ST2 | DOK7 | FAM129A |
| ENST00000378416 | CLN8 | LOC100509213 | TRAPPC6B | TEK |
| IL1B | RARRES2 | XLOC_012876 | SERPINE2 | KIF27 |
| ROR1 | ASGR2 | SPINK5 | LOC100509105 | MACF1 |
| MGP | FOXF1 | NT5E | HCST | LOC283033 |
| HIST2H3A | LOC100509541 | XLOC_I2_012871 | LOC100132850 | LOC253039 |
| AB529248 | FST | XLOC_002623 | OPCML | CBWD7 |
| THC2767054 | PSG8 | HIST1H2BK | NOV | FAM89A |
| TMEM2 | XLOC_004797 | CALU | LOC153577 | LOC340090 |
| KRTAP10-5 | CD24 | GDF15 | MT2A | CDH5 |
| UPB1 | CPA4 | XLOC_000735 | NFYA | OLR1 |
| MATN2 | MMP7 | ENST00000369161 | VIT | XLOC_I2_015220 |
| XLOC_009599 | BF106382 | HIST1H2AC | XLOC_014037 | LOC100652839 |
| SPOCK1 | SIGLEC8 | TPPP | C7orf41 | PSG1 |
| LOC100652751 | TNNT2 | LOC100507286 | HIST1H2BL | CHAC1 |
| DHX58 | MFAP5 | KRT17 | IL2RG | LOC100505921 |
| LOC100506935 | LOC100652760 | ND2 | PHGR1 | HIST2H2AA4 |
| XLOC_007352 | HERC5 | A_33_P3225552 | LOC642335 | AREG |
| LOC100507412 | C4orf51 | AGXT2 | AOX1 | LOC283214 |
| TNRC6C | FAM172A | COL4A4 | XLOC_001355 | THC2540172 |
| IFIT1 | SECTM1 | XLOC_003572 | ENST00000390461 | LOC100652849 |
| CDK11B | TCF25 | BIRC3 | CNFN | CDON |
| LOC100506172 | SNORD3B-1 | XLOC_007116 | C9orf169 | DOCK8 |
| ENST00000377803 | ECSCR | P39192 | ZMAT3 | IL32 |
| SPTBN1 | PYY | INSM1 | CCNY | TNFSF4 |
| GJB2 | XLOC_006260 | GRID2 | XLOC_009723 | C11orf44 |
| EPGN | XLOC_012592 | XLOC_008051 | KAAG1 | XLOC_001910 |
| XLOC_009765 | CRYAB | XLOC_005944 | BAZ1B | OR52B6 |
| XLOC_010490 | NPR3 | APOL6 | ING3 | SLC35F3 |
| RAMP1 | LOC100289094 | LOC100506895 | PKD2 | AGT |
| A_33_P3316671 | ADHFE1 | LOC100288814 | LOC284561 | AR |
| LY96 | XLOC_011819 | A_33_P3375496 | ENST00000425104 | CR625008 |
| CTU2 | C21orf90 | RUSC1-AS1 | CNTFR | SNORA50 |
| XLOC_004590 | THC2464556 | GIMAP1 | CFHR3 | HIST1H1C |
| XLOC_005368 | LOC100505832 | XLOC_005579 | LOC100131094 | ENST00000424852 |
| AK094933 | LOC100652782 | GBP3 | A_33_P3226492 | PID1 |
| HRASLS5 | ENST00000399211 | CSN1S1 | XLOC_004649 | A_33_P3353273 |
| XLOC_013194 | CST1 | TRIM63 | SPATA21 | SMC1A |
| XLOC_I2_013932 | NDN | GPLD1 | PRND | A_33_P3239102 |
| DKK1 | LOC100505937 | HOXD1 | KRTAP5-3 | MARCH4 |
| DENND2C | FLJ40453 | C1orf133 | PAGE3 | MLLT4 |
| XLOC_002275 | ENST00000413944 | TNFAIP3 | HIST2H4B | PSMB9 |
| TMEM132C | TDRD7 | HIST1H2AE | SOX30 | XLOC_003886 |
| BNC2 | TES | H2AFB2 | KCNC3 | PLAC1 |
| XLOC_004766 | ENST00000381524 | OR5AK2 | ENST00000413220 | HRASLS2 |
| ND5 | TGFBR2 | LOC283174 | FLJ30838 | LOC144742 |
| C3orf43 | XLOC_003595 | NANOS1 | BCO2 | RNASEK |
| COLEC11 | RBM24 | DCHS2 | LOC100130589 | XLOC_I2_010029 |
| RPH3A | DEFB132 | KRT14 | LOC100652987 | HIF3A |
| FAM19A4 | XLOC_006775 | SPTAN1 | COL13A1 | RN7SK |
| LOC100506387 | SLC22A2 | C19orf39 | XLOC_006220 | PDZK1IP1 |
| XLOC_003810 | XLOC_009604 | XLOC_I2_015132 | HIST1H2BH | PSG5 |
| ZBTB20-AS1 | HIST1H2BO | ARMS2 | LOC283516 | CARD16 |
| LINC00208 | ENST00000431422 | SERPINA4 | ZFP37 | A_33_P3277288 |
| CPO | GNGT2 | IFIH1 | HIST2H3D | XLOC_002678 |
| HIST1H2BG | ZNF226 | PSD3 | TRGV7 | XLOC_007603 |
| XLOC_001265 | OR56A5 | XLOC_013481 | LOC100653338 | COX2 |
| XLOC_010803 | MMP8 | LOC100506494 | LOC100616668 | SMTNL2 |
| SLC7A5 | TCTEX1D1 | SGK223 | PDE4DIP | TTTY13 |
| XLOC_004350 | CCL2 | DOK5 | AK124190 | CHRNB2 |
| C10orf54 | XLOC_000698 | ENST00000342995 | FAM13C | ATP6V0A4 |
| ENPP2 | PROM1 | HIST1H2BB | XLOC_005492 | LANCL3 |
| SLC38A7 | DAGLA | GAB2 | XLOC_014220 | XLOC_006952 |
| DPM3 | IL17F | ZNF605 | SNORA71B | LOC284263 |
| TP53I11 | XLOC_I2_013031 | MEGF9 | ENST00000382488 | XLOC_001598 |
| KCNJ2 | SP1 | XLOC_011294 | HIST1H4D | LOC645586 |
| ENPP6 | MAGEC2 | MIA2 | ENST00000425161 | EBF3 |
| XLOC_008024 | XLOC_010908 | A_33_P3417547 | DCDC5 | ADAT2 |
| JDP2 | XLOC_003155 | XLOC_010079 | CCL3L3 | LOC100507018 |
| XLOC_I2_006079 | ZSWIM4 | HIST1H2BI | MMP24 | XLOC_000004 |
| AF161372 | KIF25 | ATG2A | SLC25A22 | GALNT2 |
| A_33_P3334121 | XLOC_004294 | EXD1 | DOCK10 | DEPTOR |
| TCF7L1 | XLOC_I2_002952 | COL6A5 | SNORD115-48 | ZDHHC9 |
| SCN4B | XLOC_000883 | LOC100131231 | SLC15A3 | LOC340017 |
| IL15RA | APC2 | WDR86 | HIST1H2BC | S100A7A |
| XLOC_002932 | XLOC_001308 | XLOC_I2_004063 | EPB41L1 | FAT4 |
| XLOC_013772 | THC2596076 | AHRR | TMF1 | XLOC_007486 |
| ENST00000440711 | LIPH | HIST1H4F | XLOC_013477 | MT1E |
| GHDC | ENST00000391684 | HMGA2 | FER1L6-AS1 | MYH9 |
| FAM102A | LRRC18 | VDR | XLOC_I2_009884 | EMILIN2 |
| PAG1 | CHN2 | LOC100652903 | MMP12 | HIST1H2BM |
| SP2 | OR2AK2 | NLRC3 | PFN2 | LOC286189 |
| STMN2 | ENST00000390426 | EPB41L4A | MT1B | LOC284412 |
| FAS | FOLR3 | FOXI2 | BOLL | NUDT8 |
| NEURL1B | HIST1H2AD | NR1I2 | TXNDC17 | EMP1 |
| JAG2 | A_33_P3221648 | ACSL5 | ADAMTSL3 | LOC100132099 |
| C17orf78 | SQRDL | KRTAP9-3 | ALG11 | XLOC_002988 |
| USF2 | DAB2 | AATK | ZNF580 | XLOC_014030 |
| SLC39A9 | XLOC_010798 | IGSF11 | ENST00000390268 | OR2T4 |
| XLOC_013479 | XLOC_009680 | OR51B2 | ENST00000391545 | NEUROG2 |
| DENND2D | USP43 | XLOC_002317 | AK092264 | XLOC_004102 |
| LOC729626 | XLOC_005952 | SNORA71A | LOC441204 | HOXA10 |
| A_33_P3263274 | XLOC_010715 | SORCS2 | HIP1R | A_33_P3422999 |
| OR9G9 | HIST2H2BF | LOC731932 | PRKGA | XLOC_014264 |
| FAM46B | XLOC_I2_000696 | LOC440518 | XLOC_000386 | XLOC_003694 |
| BZRAP1 | SFTPA2 | XLOC_I2_004540 | PLA2G4D | PLCG2 |
| IZUMO1 | CYP1B1 | GGTA1P | XLOC_010064 | ENST00000398992 |
| PPP1R18 | SLC30A8 | SNCG | DKFZp686O1327 | XLOC_010552 |
| APOL3 | XLOC_011590 | HSPB3 | FRG2C | XLOC_008223 |
| THC2539168 | RASGRF2 | TMEM204 | XLOC_006544 | ENST00000429480 |
| A_24_P887857 | IZUMO2 | LOC100507266 | SLC26A5 | F8A1 |
| FBXO40 | MMP16 | XLOC_I2_008434 | XLOC_003260 | ATXN8OS |
| CYBRD1 | XLOC_004727 | XLOC_I2_005194 | LOC644100 | ARL11 |
| XLOC_000649 | LINC00310 | MMP10 | XLOC_009484 | TCF7L2 |
| SYCE1 | SNAR-G1 | XLOC_013364 | XLOC_008072 | GNA11 |
| XLOC_006597 | ADIPOQ | XLOC_I2_000706 | THC2624074 | DLX6 |
| BANF1 | XLOC_006680 | ZNF433 | LOC646890 | XLOC_012073 |
| SH3PXD2B | XLOC_I2_007074 | XLOC_002653 | MT1L | TRIML2 |
| PIWIL1 | MYEF2 | SLC37A3 | LOC100652804 | PSME3 |
| OR8G1 | COX1 | XLOC_005053 | CBX5 | LOC649395 |
| NUPR1 | ITGB8 | LOC541471 | CBLN3 | GP6 |
| LOC100653033 | IL6 | OR51G1 | C6orf222 | EDAR |
| ENST00000439423 | ND4L | TRIM29 | FLJ44313 | SMTN |
| LOC400684 | XLOC_I2_003073 | A_33_P3276604 | LOC100653004 | XLOC_013413 |
| PASD1 | ATP8 | XLOC_002194 | LOC100506403 | BPIFB3 |
| XLOC_002585 | XLOC_010703 | XLOC_013031 | LOC729177 | TRIL |
| XLOC_I2_005705 | XLOC_010054 | OSMR | LOC390877 | MGAT4B |
| OR2T11 | POU3F3 | SFXN1 | MPL | XLOC_011010 |
| XLOC_I2_011899 | ANKRD7 | A_33_P3251916 | EBI3 | ERVFRD-1 |
| APOL1 | WASH3P | XLOC_I2_011870 | LOC100505959 | XLOC_009548 |
| HINT3 | HCRTR2 | GPR126 | MYOF | MYZAP |
| XLOC_011980 | OSCAR | XLOC_010410 | LOC100526771 | XLOC_001007 |
| DDX18 | XLOC_I2_006173 | PTPN13 | XLOC_009301 | ENST00000317656 |
| XLOC_007956 | HMX1 | RNU2-2 | C9orf152 | XLOC_007801 |
| FOLR1 | PLAC8 | LOC730236 | XLOC_006485 | PCDHB6 |
| XLOC_I2_010270 | C1QTNF6 | XLOC_006779 | GTF2H5 | XLOC_I2_004817 |
| XLOC_010268 | NP1243929 | LOC100131131 | TTI2 | AZU1 |
| AK130931 | RNF213 | NP106737 | XLOC_008143 | HIST1H2BD |
| ZNF704 | HNRNPUL1 | PROCR | PGCP | XLOC_I2_013484 |
| LOC100506898 | XLOC_002151 | XLOC_013932 | XLOC_008999 | DUSP8 |
| PFN1P2 | XLOC_004612 | SVIL | SLC12A7 | NOS2 |
| OCR1 | XLOC_007586 | FLJ23152 | MS4A10 | SPINK6 |
| MS4A1 | XLOC_I2_009578 | A_33_P3365963 | LOC100653060 | XLOC_003020 |
| XLOC_001414 | XLOC_010252 | BGN | XLOC_005989 | XLOC_013348 |
| TTPA | C5orf42 | PEG10 | MRPL2 | CYP4F30P |
| ENST00000526741 | XLOC_009208 | A_33_P3245131 | EPPK1 | ENST00000447197 |
| OR8G5 | NEUROD1 | IGFBP7 | LARP6 | GPS2 |
| XLOC_006936 | XLOC_001367 | AK123255 | SIGLEC6 | AF119900 |
| SNORD111 | AKAP5 | FBXO27 | SYNPO2L | VEPH1 |
| GAS2L3 | SYK | SMARCA2 | OVOL1 | WFS1 |
| CHRM5 | DLX3 | LOC100507392 | XKRY2 | XLOC_012047 |
| LEP | CROT | CREB3L2 | XLOC_012513 | ENST00000414116 |
| A_33_P3368920 | LOC100132354 | AGR3 | AK027069 | A_33_P3408665 |
| FUT1 | DMRTA2 | XLOC_006389 | OR51A7 | THC2731377 |
| FLJ37644 | GRAMD3 | LOC100506922 | SLC22A5 | PKD2L2 |
| LYZL4 | NCSTN | XLOC_010684 | PHEX | CD200 |
| SLC39A4 | LMO2 | DGAT2L7 | XLOC_I2_000423 | ARHGDIB |
| LOC100131180 | XLOC_004474 | LOC284578 | SNORD114-23 | ATF6 |
| LOC100506800 | MFI2 | BTN1A1 | PDE6H | ZNF287 |
| KCNV2 | THC2721084 | GNRHR2 | CYP4Z1 | C8orf4 |
| MYRIP | LAMB1 | FBXO39 | XLOC_012365 | A_33_P3300591 |
| XLOC_I2_015397 | XLOC_000527 | CPEB1 | FAM201A | GJA1 |
| DMD | APBB1IP | ST6GALNAC1 | SH3GL2 | C11orf74 |
| XLOC_I2_013149 | XLOC_009912 | IRX4 | XLOC_008157 | APOB |
| RHOB | SLC2A12 | XLOC_003313 | DNAJB3 | SVEP1 |
| CORO2B | SBK1 | FASLG | LOC284215 | LOC400680 |
| NR5A2 | LOC100506130 | XLOC_012716 | SLC26A2 | XLOC_007750 |
| ZNF578 | LRRTM3 | AB529247 | XLOC_010682 | ACVRL1 |
| KCNS3 | CNPY4 | XLOC_000324 | VANGL2 | RELB |
| ITGA6 | LOC642929 | DCDC2B | CDK6 | XLOC_007860 |
| NP414419 | XLOC_000299 | LOC100131607 | SLC6A4 | XLOC_011540 |
| XLOC_008297 | XLOC_I2_008200 | LOC100506085 | CDH6 | CCDC79 |
| XLOC_I2_008632 | LOC100289255 | AP3S1 | CB215009 | LEPROT |
| OPTN | TMEM143 | UBIAD1 | HMGCS2 | GPRC6A |
| BC104424 | GALNT4 | TIMM17B | IDO1 | EDN1 |
| HEY2 | XLOC_I2_013886 | CMIP | OLFML1 | ENST00000502368 |
| XLOC_003734 | SNORD116-26 | NR2F1 | KIAA0247 | XLOC_002125 |
| ND3 | A_33_P3345808 | A_33_P3328958 | MAOA | PHOX2A |
| XLOC_002908 | LOC401286 | LINC00487 | CHRFAM7A | HIST1H4I |
| DKFZP547L112 | XLOC_010264 | TSPAN12 | ATP4B | LOC100653120 |
| PEA15 | LOC100129112 | XLOC_I2_015536 | XLOC_011193 | ALPK2 |
| WIZ | NPB | LOC100131366 | XLOC_I2_013437 | ARHGAP40 |
| SPATA20 | XLOC_000737 | XLOC_I2_012154 | XLOC_010948 | SNTN |
| SNORD92 | TRIM77P | ENST00000548231 | SLC8A2 | ZC3H12C |
| A_33_P3404739 | LOC100507025 | XLOC_008559 | XLOC_009891 | XLOC_008693 |
| AK023309 | XLOC_I2_009500 | IL1R2 | XLOC_014368 | SIGLEC16 |
| ENST00000555882 | THC2566752 | FAM166A | GPR174 | XLOC_000616 |
| LOC100133089 | VTN | FLJ13224 | PCSK7 | XLOC_006780 |
| ND4 | XLOC_013921 | XLOC_000842 | LOC100506791 | ENST00000520426 |
| CHRM1 | XLOC_I2_004072 | FLJ11292 | THC2529564 | XLOC_009818 |
| TNFRSF8 | FLJ40194 | SNHG11 | CCDC9 | LOC284108 |
| KLK1 | UNC45B | RPL23AP64 | SMARCD1 | XLOC_005755 |
| NCF2 | PNPLA4 | AVPR1A | XLOC_013206 | CLCA2 |
| EBNA1BP2 | NMUR2 | SULF1 | CYS1 | LOC100653000 |
| EMP3 | GABRR2 | LOC100506610 | SUZ12P | DKFZP434K028 |
| XLOC_007218 | SRGAP1 | ASTL | A_33_P3227661 | CGN |
| INF2 | LOC644838 | LOC100506591 | BRP44L | XLOC_009336 |
| LOC648987 | PRG4 | MECOM | FAM24B | C12orf34 |
| LOC100507475 | XLOC_I2_007070 | XLOC_012601 | A19P00812033 | XLOC_006876 |
| XLOC_I2_009508 | LOC100507303 | NKX2-4 | LOC100131262 | CTNND1 |
| BM544686 | EEPD1 | DNAJB1 | LOC100133612 | CXCL10 |
| TM9SF3 | PSCA | DMRTB1 | XLOC_002073 | XLOC_007088 |
| XLOC_014349 | PRRG3 | LOC388630 | PRICKLE2 | LOC100506714 |
| XLOC_000027 | LINC00304 | FTO | SOX1 | IFITM2 |
| IL23R | LOC100505899 | IL28RA | LOC91948 | XLOC_006911 |
| LCP1 | XLOC_011957 | XLOC_000225 | XLOC_014251 | XLOC_001506 |
| ZSCAN23 | AK5 | MBP | XLOC_008252 | CDH1 |
| XLOC_012335 | RHEBL1 | CSNK1G2 | MGC45922 | XLOC_011655 |
| A_33_P3373014 | XIRP2 | XLOC_I2_013963 | TBC1D2 | CAMK2N1 |
| A_33_P3213311 | DMPK | LOC100128857 | XLOC_I2_012788 | XLOC_006377 |
| OTUD3 | XLOC_005377 | ENST00000416673 | NUDT3 | SNORD114-15 |
| XLOC_I2_009181 | XLOC_001303 | A_33_P3313625 | HRCT1 | IFNGR1 |
| KRTAP4-11 | XLOC_005620 | XLOC_011057 | MAST4 | SYBU |
| KLRC1 | XLOC_007955 | XLOC_I2_004342 | C14orf166B | XLOC_003135 |
| KIAA1462 | TNNC1 | XLOC_006752 | NAAA | HDC |
| XLOC_004342 | UTRN | KIAA1598 | XLOC_001749 | LOC100652762 |
| XLOC_006176 | XLOC_I2_007452 | LOC100507317 | SCN10A | PRR4 |
| ENST00000436042 | HOXB1 | XLOC_001470 | LPAR6 | XLOC_I2_011281 |
| LOC100507319 | NEDD8 | A_24_P273043 | WNT16 | ENST00000419160 |

TABLE 3
Downregulated Genes in PARP13 siRNA cells (Log2FC > 1)

| | | | | |
|---|---|---|---|---|
| TNXB | FBLN2 | ENO3 | ALDOC | PRSS8 |
| FAM3B | HYMAI | BC028053 | TFF3 | XLOC_I2_000917 |
| H19 | NDUFA4L2 | NFE2 | CFI | NTS |
| LOC100506810 | CA9 | SPINK4 | CALHM3 | |

TABLE 4
Upregulated Genes in PARP13 siRNA cells (Log2FC > 1)

| | | | | |
|---|---|---|---|---|
| CCL5 | TNFRSF10D | OASL | GSTP1 | IFIT2 |
| CLDN18 | GPNMB | C14orf64 | RARRES3 | XLOC_007808 |
| XLOC_003310 | DDX58 | IFIT3 | ISG15 | XLOC_014103 |
| PI3 | ELSPBP1 | COL8A1 | CFH | LOC100506923 |
| IFI44 | LOC100127961 | HS3ST2 | DOK7 | FAM129A |
| ENST00000378416 | CLN8 | LOC100509213 | TRAPPC6B | TEK |
| IL1B | RARRES2 | XLOC_012876 | SERPINE2 | KIF27 |
| ROR1 | ASGR2 | SPINK5 | LOC100509105 | MACF1 |
| MGP | FOXF1 | NT5E | HCST | LOC283033 |
| HIST2H3A | LOC100509541 | XLOC_I2_012871 | LOC100132850 | LOC253039 |
| AB529248 | FST | XLOC_002623 | OPCML | CBWD7 |
| THC2767054 | PSG8 | HIST1H2BK | NOV | FAM89A |
| TMEM2 | XLOC_004797 | CALU | LOC153577 | LOC340090 |
| KRTAP10-5 | | | | |

TABLE 5
Downregulated Genes in PARP13 knockout cell lines (Cutoff
p-value < 0.01, log fold change of expression of 1)

| | | | | |
|---|---|---|---|---|
| RIMS1 | EVPLL | FBLIM1 | LOC100505633 | LINC00052 |
| AGR2 | INPP5D | ETV5 | LGALS8 | SGK223 |
| FAM213A | FBXO2 | CDH12 | LOC728463 | PROS1 |
| ENST00000425609 | DSCAM-AS1 | CFH | PPFIA4 | XLOC_008339 |
| RNF182 | C16orf62 | ALCAM | A_33_P3373985 | TRAPPC3L |
| FGF7 | CPLX3 | E1A_r60_a107 | ICAM2 | TNNC1 |
| SEPT11 | ENST00000426068 | KHDC1 | ENST00000561392 | LRRC61 |
| KGFLP1 | DUSP6 | QPRT | THC2548955 | SLC9A3R2 |
| XLOC_014512 | XLOC_000642 | ANGPTL4 | RN7SK | RASGEF1A |
| HOXB13 | ZC3HAV1 | FGFR2 | GCGR | IGFBP3 |
| E1A_r60_n9 | POU5F1 | SPINK4 | XLOC_007215 | CRABP2 |
| AATK | ADAMTS4 | PPIC | DNAH17-AS1 | HSPB7 |
| SPINK5 | WFDC1 | ENST00000558081 | PPP1R3F | PIP5KL1 |
| ENST00000591313 | ENST00000442712 | TFF2 | C1orf35 | NCCRP1 |
| NTS | COL6A1 | SYTL4 | H19 | GAL3ST1 |
| LINC00665 | GPM6B | A_21_P0014749 | CASKIN1 | ENST00000447535 |
| HKDC1 | E1A_r60_n11 | CMAHP | TAF5L | PFKFB4 |
| DNAH2 | DPPA2 | HIST2H3A | ALDH3A1 | ENST00000456721 |
| MME | ACTL8 | NDUFA4L2 | ENST00000552826 | ENST00000430647 |
| SLC12A3 | CA9 | IL18R1 | TCAM1P | IL32 |
| MFAP5 | E1A_r60_a135 | FZD4 | MEOX1 | MYEOV |
| THBD | MAGIX | ENST00000551539 | ENST00000591384 | SYDE1 |
| RPRM | GYG2 | FABP3 | ALPP | SLC2A6 |
| C8orf22 | PLCB2 | DEPDC5 | A_21_P0014771 | DUSP4 |
| PTN | RUSC1-AS1 | FAM20A | LCN15 | DYSF |
| C6orf147 | ENST00000377803 | MAPK4 | LINC00476 | IER3 |
| SPRY1 | ADAMTSL1 | SNORD10 | SNORD3B-1 | OLFM1 |
| A_21_P0014880 | HPD | HIST1H1B | NME4 | AV738989 |
| RNA28S5 | FOSL1 | BX648590 | SIMC1 | AF161372 |
| TIMM17B | ZNF124 | SUV39H1 | KIAA1211L | A_33_P3375496 |
| AK124190 | BIK | CA5BP1 | CCL26 | SET |
| TSPAN14 | CBX1 | JMJD8 | FAM195A | ZNF467 |
| SUSD2 | HIST1H3F | HN1L | NET1 | SCARNA22 |
| ATF5 | RNA18S5 | CTPS2 | PLCXD1 | RASSF9 |
| ALKBH5 | STRA6 | RNF32 | CSK | LOC101929120 |
| TFF1 | TRIB3 | PIM2 | OR4D9 | A_33_P3314574 |
| ADRB2 | PPEF1 | HIST1H2BF | HIST1H3H | ZNF689 |
| DDX17 | HIST1H4A | TMBIM1 | RBM3 | SNORA34 |
| NGEF | RCOR2 | COL9A2 | IQSEC2 | SNORA73A |
| SRPX2 | PQBP1 | FJX1 | PRCC | IGSF9 |
| CTNNB1 | MUC13 | PTGS1 | TIMP3 | AK125205 |
| HIST1H2AM | PRICKLE3 | SRCAP | ISL1 | SNCG |
| FOXRED2 | BCO2 | PTK6 | A_21_P0014763 | MYOM2 |
| FAM83F | XK | HQ013231 | IGSF8 | LOC284751 |
| XLOC_001173 | XLOC_005220 | TFE3 | MOCOS | LOC100507412 |
| ENST00000433232 | TAC3 | PLEKHA6 | EMILIN1 | RBBP4 |
| PI3 | XLOC_010776 | ARHGDIB | ARHGEF35 | ENST00000445293 |
| PCDH1 | ETV4 | IL1RL1 | F2RL1 | ALG1L2 |
| C9orf152 | FRMD3 | PHKA2 | PDE1C | FAM166A |
| ASXL1 | LOC100505664 | TGFB2 | GJB5 | CENPW |
| PTGER4 | ALG1L | BCHE | ZFP82 | IQCD |
| SLC16A4 | HIST4H4 | FAM13A | A_33_P3316671 | FBXO44 |
| BCL6B | AF390550 | C1orf226 | HAPLN2 | XLOC_006291 |
| CHAC1 | MAP2K3 | BRINP3 | XLOC_001532 | ENST00000376775 |
| AKT3 | KCNJ18 | AY927536 | CYP4X1 | GALNT5 |
| LOC284581 | GLUL | PLEKHG1 | SCNN1A | USP21 |
| JAG2 | SNORA71D | CNTD1 | THC2680609 | SLC35A2 |
| CT45A5 | LMCD1 | PLAC8 | LRG1 | PAQR4 |
| PRR22 | LOC100130430 | SMC1A | HIST3H2BB | BQ719082 |

TABLE 6
Upregulated Genes in PARP13 knockout cell lines (Cutoff
p-value < 0.01, log fold change of expression of 1)

| | | | | |
|---|---|---|---|---|
| MAGEB2 | LOC728392 | AIG1 | FAR2P1 | CFB |
| PLAG1 | CNRIP1 | FBXO32 | HRK | NUDT12 |
| ABCC6 | TPST1 | LGSN | PDK4 | ABLIM2 |
| EBF1 | CFI | DCP_22_2 | GPCPD1 | BDKRB2 |
| HORMAD1 | TNFRSF10D | DCP_22_0 | DNAJC12 | FGL1 |
| LOC101060810 | C11orf53 | CGA | NCK2 | HIST2H2AC |
| E1A_r60_a22 | DCP_22_6 | IL6 | PLEKHA4 | TPPP |
| OGDHL | DCP_22_4 | C1R | DCP_22_7 | C1S |
| XLOC_014103 | BDKRB1 | CHMP1B | GLIS1 | LMO2 |
| LOC729041 | CHRD | PRG4 | KIAA1244 | PLCG2 |
| LOC100506688 | TMEM54 | LOC101926940 | MFSD6 | SPTLC3 |
| MCF2L | SRRM3 | GPR160 | AK091028 | REEP1 |
| IFI44 | HAPLN3 | CLYBL | NFE2 | LAMP2 |
| VAV3 | C4B | DAB2 | SERPINB3 | IFIT2 |
| ENST00000511182 | AMDHD1 | RORC | TRABD2B | AK4 |
| ENST00000435913 | AMY1C | SMARCA1 | ENST00000432361 | NNT |
| EPHB2 | C21orf90 | NCAM2 | ISG15 | RIC8B |
| KLHL41 | EDA2R | BMF | LINC00641 | STOX1 |
| LOC285696 | ANKRD20A12P | SPTSSA | DDX60 | CTU2 |
| OASL | MCTP1 | GPR87 | LINC00673 | SAT1 |
| LINC00847 | NKD2 | CD59 | WDR54 | MYH10 |
| CNNM2 | EGR1 | CDHR4 | CDH16 | ZADH2 |
| ENST00000433342 | OTUD7A | MMP12 | TDRKH | CLIC4 |
| MAPT | CHCHD7 | FAM167B | THC2502236 | IL13RA1 |
| LOC338620 | CLK1 | TGFBI | DUSP1 | KRT16P2 |
| TMEM190 | MPP1 | C5orf56 | ETS2 | UACA |
| LDOC1 | NOV | NFYA | EMR2 | MFF |
| NLRP1 | ENST00000416502 | PRKAA1 | MTURN | NEDD4L |
| EEPD1 | ITM2C | PAGE3 | RPL23AP7 | LOC100506548 |
| ZMIZ1-AS1 | LINC01060 | LOC100130476 | TSPAN3 | NR3C2 |
| FAR2P2 | FAM172A | USP47 | ENST00000578664 | RPL22 |
| REPS2 | MAN1C1 | LRRC8B | ZNF572 | DCN |
| C11orf74 | VIT | ANXA10 | PLA2G12A | JMY |
| GPNMB | ZAK | SMPX | KRT17 | CPEB4 |
| NNT-AS1 | DNMT3L | XLOC_010544 | TTC7B | TECPR2 |
| SERPINB4 | TDRD7 | SEMA4F | PAM | SOCS2 |
| PID1 | SDSL | SUB1 | GNAZ | USP33 |
| LEPR | RAB27A | XLOC_014512 | SLC12A7 | PRSS23 |
| TP53INP1 | C1QL4 | PSG8 | LAMTOR5-AS1 | NANOS1 |
| B3GALT4 | DNAJC21 | LINC00239 | SNX10 | PIGZ |
| GSAP | LINC00882 | LSMEM1 | HCG11 | PRKGA |
| MLLT4 | RARRES3 | RASL10B | FAM189A2 | GRID1-AS1 |
| UST | DNER | SERPINB1 | TCAIM | C5orf34 |
| SAMD12 | BRIX1 | RIMS3 | XLOC_000865 | CHN1 |
| RASSF4 | PDE2A | ID2 | CTSK | C8orf4 |
| ENST00000582047 | VIP | NEAT1 | APPBP2 | C10orf10 |
| PGM2L1 | MBNL2 | PCSK1 | ENST00000432250 | BC025320 |
| NUDT7 | KLHDC7B | LYRM5 | | |

The invention provides methods of modulating expression (e.g., mRNA and/or protein) and/or activity of any of the target genes listed in Tables 1-6 by administering a PARP13 activator that binds specifically to PARP13 to increase PARP13 activity and/or interaction or binding to any of the target gene transcripts listed in Tables 1-6. The activity of PARP13 may be an increase in the poly-ADP-ribosylation of one or more (e.g., 1, 2, 3, 4, or 5) target gene(s) (e.g., any of the genes listed in Tables 1-6). Additional activities of a PARP protein are described herein. In these methods, one or more PARP13 activators preferably increase (e.g., at least 10%, 15%, 20%, 30%, 40%, 50%, 60%, 70%, 80%, 90%, or 100%) the expression (e.g., mRNA and/or protein) and/or activity of any of the target genes listed in Tables 1 and 3 that are downregulated. In other methods, one or more PARP13 activators preferably decrease (e.g., by at least by at least 10%, 20%, 30%, 40%, 50%, 60%, 70%, 80%, 90%, or 100%) the expression (e.g., mRNA and/or protein) and/or activity of any of the target genes listed in Tables 2 and 4 that are upregulated.

### Conditions and Disorders

Disorders Associated with Immune Misregulation

Many diseases and syndromes are associated with immune misregulation and involve misregulation of RNA transcripts important in immunomodulatory signaling pathways. Immune misregulation can contribute to cancer, inflammation, autoimmunity, neurological disorders, developmental syndroms, diabetes, cardiovascular disease, among others. The compositions of the invention is envisioned to be useful for treating disorders associated with immune misregulation, for example, autoinflammatory diseases. Autoinflammatory diseases include, but are not limited to, familian Mediterranean fever (FMF), neonatal onset multisystem inflammatory disease (NOM ID), tumor necrosis factor (TNF) receptor-associated period syndrome (TRAPS), deficiency fo the interleukin-1 receptor antagonist (DIRA), and Behcet's disease.

### Autoimmune Disorders

The compositions of the invention can be used to treat autoimmune disorders. Autoimmune diseases include but are not limited to systemic lupus erythematosus (SLE), CREST syndrome (calcinosis, Raynaud's syndrome, esophageal dysmotility, sclerodactyl, and telangiectasia), opsoclonus, inflammatory myopathy (e.g., polymyositis, dermatomyositis, and inclusion-body myositis), systemic scleroderma, primary biliary cirrhosis, celiac disease (e.g., gluten sensitive enteropathy), dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, LambertEaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and Neuromyelitis optica.

Other autoimmune disorders include pernicious anemia, Addison's disease, psoriasis, inflammatory bowel disease, psoriatic arthritis, Sjögren's syndrome, lupus erythematosus (e.g., discoid lupus erythematosus, drug-induced lupus erythematosus, and neonatal lupus erythematosus), multiple sclerosis, and reactive arthritis.

Additional disorders that may be treated using the methods of the present invention include, for example, polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, alopecia arcata, pemphigoid, scleroderma, progressive systemic sclerosis, adult onset diabetes mellitus (e.g., type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

### Viral and Virus-Associated Disorders

The methods and compositions of the invention can be used to treat and/or prevent viral infections and/or virus-associated disorders. The virus causing the infection can be a member of the herpes virus family, a human immunodeficiency virus, parvovirus, or coxsackie virus. A member of the herpes virus family can be herpes simplex virus, herpes genitalis virus, varicella zoster virus, Epstein-Barr virus, human herpesvirus 6, or cytomegalovirus. The methods and compositions described herein can be used to treat and/or prevent infections caused by any virus, including, for example, Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus; ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian rnyeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barman Forest virus, Bebaru virus, Berrirnah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Caicivirus, California encephalitis virus, camelpox virus, canarypox virus, canid herpesvirus, canine coronavirus, canine distemper virus, canine herpesvirus, canine minute virus, canine, parvovirus, Cana Delgadito virus, caprine arthritis virus, caprine encephalitis virus, Caprine, Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, CharleviLle virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious ecthyma virus, contagious pustular dermatitis virus, Coronavirus, Corriparta virus, coryza virus, cowpox virus, coxsackie virus, CFA/(cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine rnorbillivirus, equine rhinopneumonitis virus, equine rhinovirus, Eubenangu virus, European elk papillornavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowipox virus, Friend virus, Gammaretrovirus, GB hepatitis virus, GB virus, German measles virus, Getah virus, Gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, Homy (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvrus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human Blymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, human papillomavirus, human T cell leukemia virus, human T cell leukemia virus I, human T cell leukemia virus II, human T cell leukemia virus III, human T cell lymphoma virus human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elaon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phiebovirus, phocine distemper virus, Picodnavirus, Picornavirus, pig cytomegalovirus pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect HiH virus, Provirus, pseudocowpox virus, pseudorabies virus, psittacinepox virus, quailpox virus, rabbit fibroma virus, rabbit kidney vacuolating virus, rabbit papillomavirus, rabies virus, raccoon parvovirus, raccoonpox virus, Ranikhet virus, rat cytomegalovirus, rat parvovrus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), seaipox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type STLV (simian T lymphotropic virus) type stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolatina virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovrus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus.

Types of virus infections and related disorders that can be treated include, for example, infections due to the herpes family of viruses such as EBV, CMV, HSV I, HSV II, VZV and Kaposi's-associated human herpes virus (type 8), human T cell or B cell leukemia and lymphoma viruses, adenovirus infections, hepatitis virus infections, pox virus infections, papilloma virus infections, polyoma virus infections, infections due to retroviruses such as the HTLV and HIV viruses, and infections that lead to cellular disorders resulting from and/or associated with viral infection such as, for example, Burkitt's lymphoma, EBV-induced malignancies, T and B cell lymhoproliferative disorders and leukemias, and other viral-induced malignancies. Other neoplasias that can be treated include virus-induced tumors, malignancies, cancers, or diseases that result in a relatively autonomous growth of cells. Neoplastic disorders include leukemias, lymphomas, sarcomas, carcinomas such as a squamous cell carcinoma, a neural cell tumor, seminomas, melanomas, germ cell tumors, undifferentiated tumors, neuroblastomas (which are also considered carcinomas by some), mixed cell tumors, or other malignancies. Neoplastic disorders prophylactically or therapeutically treatable with compositions of the invention include small cell lung cancers and other lung cancers, rhabdomyosarcomas, chorio carcinomas, glioblastoma multiformas (brain tumors), bowel and gastric carcinomas, leukemias, ovarian cancers, prostate cancers, osteosarcomas, or cancers that have metastasized. Diseases of the immune system that are treatable include Hodgkins' disease, the non-Hodgkin's lymphomas including the follicular and nodular lymphomas, adult T and B cell and NK lymphoproliferative disorders such as leukemias and lymphomas (benign and malignant), hairy-cell leukemia, hairy leukoplakia, acute myelogenous, lymphoblastic or other leukemias, chronic myelogenous leukemia, and myelodysplastic syndromes. Additional diseases that can be treated or prevented include breast cell carcinomas, melanomas and hematologic melanomas, ovarian cancers, pancreatic cancers, liver cancers, stomach cancers, colon cancers, bone cancers, squamous cell carcinomas, neurofibromas, testicular cell carcinomas, kidney and bladder cancers, cancer and benign tumors of the nervous system, and adenocarcinomas.

### Combination Therapy

The compositions described herein can be formulated or administered in combination with an immunosuppressant. Examples of immunosuppressants include, but are not limited to, calcineurin inhibitors (e.g., cyclosporin A (Sandimmune®), cyclosporine G tacrolimus (Prograf®, Protopic®)), mTor inhibitors (e.g., sirolimus (Rapamune®, Neoral®), temsirolimus (Torisel®), zotarolimus, and everolimus (Certican®)), fingolimod (Gilenya™), myriocin, alemtuzumab (Campath®, MabCampath®, Campath-1H®), rituximab (Rituxan®, MabThera®), an anti-CD4 monoclonal antibody (e.g., HuMax-CD4), an anti-LFA1 monoclonal antibody (e.g., CD11a), an anti-LFA3 monoclonal antibody, an anti-CD45 antibody (e.g., an anti-CD45RB antibody), an anti-CD19 antibody (see, e.g., U.S. Patent Publication 2006/0280738), monabatacept (Orencia®), belatacept, indolyl-ASC (32-indole ether derivatives of tacrolimus and ascomycin), azathioprine (Azasan®, Imuran®), lymphocyte immune globulin and anti-thymocyte globulin [equine] (Atgam®), mycophenolate mofetil (Cellcept®), mycophenolate sodium (Myfortic®), daclizumab (Zenapax®), basiliximab (Simulect®), cyclophosphamide (Endoxan®, Cytoxan®, Neosar™, Procytox™ Revimmune™), prednisone, prednisolone, leflunomide (Arava®), FK778, FK779, 15-deoxyspergualin (DSG), busulfan (Myleran®, Busulfex⁸), fludarabine (Fludara®), methotrexate (Rheumatrex®, Trexall®), 6-mercaptopurine (Purinethol®), 15-deoxyspergualin (Gusperimus), LF15-0195, bredinin, brequinar, and muromonab-CD3 (Orthoclone®).

Methods for assessing immunosuppressive activity of an agent are known in the art. For example, the length of the survival time of the transplanted organ in vivo with and without pharmacological intervention serves as a quantitative measure for the suppression of the immune response. In vitro assays may also be used, for example, a mixed lymphocyte reaction (MLR) assay (see, e.g., Fathman et al., J. Immunol. 118:1232-8, 1977); a CD3 assay (specific activation of immune cells via an anti-CD3 antibody (e.g., OKT3)) (see, e.g., Khanna et al., Transplantation 67:882-9, 1999; Khanna et al. (1999) Transplantation 67:S58); and an IL-2R assay (specific activation of immune cells with the exogenously added cytokine IL-2) (see, e.g., Farrar et al., J. Immunol. 126:1120-5, 1981).

Cyclosporine A (CsA; CAS No. 59865-13-3; U.S. Pat. No. 3,737,433) and its analogs may be used as an immunosuppressant. A number of other cyclosporines and their derivatives and analogs that exhibit immunosuppressive activity are known. Cyclosporines and their formulations are described, for example, in 2004 Physicians' Desk Reference® (2003) Thomson Healthcare, 58th ed., and U.S. Pat. Nos. 5,766,629; 5,827,822; 4,220,641; 4,639,434; 4,289,851; 4,384,996; 5,047,396; 4,388,307; 4,970,076; 4,990,337; 4,822,618; 4,576,284; 5,120,710; and 4,894,235.

Tacrolimus (FK506) is a macrolide which exerts effects largely similar to CsA, both with regard to its molecular mode of action and its clinical efficacy (Liu, Immunol. Today 14:290-5, 1993; Schreiber et al., Immunol. Today, 13:136-42, 1992); however, these effects are exhibited at doses that are 20 to 100 times lower than CsA (Peters et al., Drugs 46:746-94, 1993). Tacrolimus and its formulations are described, for example, in 2004 Physicians' Desk Reference® (2003) Thomson Healthcare, 58th ed., and U.S. Pat. Nos. 4,894,366; 4,929,611; and 5,164,495.

Sirolimus (rapamycin) is an immunosuppressive lactam macrolide produceable, for example, by Streptomyces hygroscopicus. Numerous derivatives of sirolimus and its analogs and their formulations are known and described, for example, in 2004 Physicians' Desk Reference® (2003) Thomson Healthcare, 58th ed., European Patent EP 0467606; PCT Publication Nos. WO 94/02136, WO 94/09010, WO 92/05179, WO 93/11130, WO 94/02385, WO 95/14023, and WO 94/02136, and U.S. Pat. Nos. 5,023,262; 5,120,725; 5,120,727; 5,177,203; 5,258,389; 5,118,677; 5,118,678; 5,100,883; 5,151,413; 5,120,842; and 5,256,790.

The compositions described herein can also be formulated or administered in combination with an antiviral agent. Antiviral agents can be selected from the group consisting of: an interferon, an amino acid analog, a nucleoside analog, an integrase inhibitor, a protease inhibitor, a polymerase inhibitor, and a transcriptase inhibitor. Other antiviral agents include, but are not limited to: abacavir, acyclovir, acyclovir, adefovir, amantadine, amprenavir, ampligen, arbidol, atazanavir, atripia, balavir, bocepreviretet, cidofovir, combivir, dolutegravir, darunavir, delavirdine, didanosine docosanol, edoxudine, efavirenz, erntricitabine, enfuvirtide, entacavir, ecoliever, famciclovir, fornivirsen, fosarnprenavir, foscarnet, fosfonet, fusion inhibitor, ganciciovir, ibacitabine, irnunovir, idoxuridine, imiquirnod, indinavir, inosine, interferon type III, interferon type II, interferon type I, interferon, lamivudine, lopinavir, loviride, maraviroc, rnoroxydine, methisazone, nelfinavir, nevirapine, nexavir, oxeitamivir, peginterferon α-2a, penciciovir, peramivir, pleconaril, podophyliotoxin, raitegravir, ribavirin, rimantadine, ritonavir, pyramidine, saquinavir, sofosbuvir, tea tree oil, teiaprevir, tenofovir, tenofovir disoproxil, tipranavir, trifluridine, trizivir, trornantadine, truvada, traporved, valaciclovir, valganciciovir, vicriviroc, vidarabine, viramidine, zaicitabine, zanamivir, and zidovudine.

### Administration and Dosage

The present invention also relates to pharmaceutical compositions that contain one or more PARP13 activators or a combination of a PARP13 activator and a therapeutic agent (e.g., a combination of a PARP13 activator and an antiviral agents, immunosuppressants, and/or anticancer agents). The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer, Science 249:1527-1533, 1990.

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a patient with a clinically determined predisposition or increased susceptibility to development of a tumor or cancer. Compositions of the invention can be administered to the patient (e.g., a human) in an amount sufficient to delay, reduce, or preferably prevent the onset of clinical disease or tumorigenesis. In therapeutic applications, compositions are administered to a patient (e.g., a human) already suffering from a cancer in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of cancer, an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. The agents of the invention are administered to a subject (e.g. a mammal, such as a human) in an effective amount, which is an amount that produces a desirable result in a treated subject (e.g. the slowing or remission of a cancer or neurodegenerative disorder). Such therapeutically effective amounts can be determined empirically by those of skill in the art.

The patient may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A patient may also receive an agent of the composition in the range of 0.1 to 3,000 mg per dose once every two or three weeks.

Single or multiple administrations of the compositions of the invention comprising an effective amount can be carried out with dose levels and pattern being selected by the treating physician. The dose and administration schedule can be determined and adjusted based on the severity of the disease or condition in the patient, which may be monitored throughout the course of treatment according to the methods commonly practiced by clinicians or those described herein.

The compounds and formulations of the present invention may be used in combination with either conventional methods of treatment or therapy or may be used separately from conventional methods of treatment or therapy. When the compounds and formulations of this invention are administered in combination therapies with other agents, they may be administered sequentially or concurrently to an individual. Alternatively, pharmaceutical compositions according to the present invention include a combination of a compound or formulation of the present invention in association with a pharmaceutically acceptable excipient, as described herein, and another therapeutic or prophylactic agent known in the art.

The formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

The following examples are to illustrate the invention. They are not meant to limit the invention in any way.

### EXAMPLES

### Materials and Methods

Experiments were performed in HeLa Kyoto cells unless otherwise stated. Knockdowns were performed using Lipofectamine 2000 as per manufacturer's instructions with double transfections of 48 h. Exogenously expressed constructs were transfected using Lipofectamine 2000 for 24 h before the assay. Mutants were cloned using GeneString technology. TRAILR4 3′UTR was cloned from Origene clone SC117708 into psiCHECK2 using Gene String technology. TRAILR4 3′UTR fragments were cloned by PCR amplification of the indicated regions and cloned into psiCHECK2. Renilla and Firefly luminescence were measured 48 h post transfection. Crosslinking followed by immunoprecipitation was performed as previously described in Leung et al. (Nature structural &molecular biology. 18: 237-244, 2011). To assess cell sensitivity to TRAIL-mediated apoptosis, cells were treated with TRAIL for 24 h, and cell viability was assayed by MTT assay (Millipore) or by Annexin V/PI flow cytometry (Biolegend) as per manufacturer's instructions. Standard Western Blotting techniques were used.

### Cell Culture and Transfection

Cells were grown at 37 C and 5% CO₂. HeLa Kyoto (ATCC), SW480 (a gift from Ryoma Ohi, Vanderbilt), and HEK293 (ATCC) cells were maintained in DMEM (Invitrogen) supplemented with 10% Fetal Bovine Serum (Life technologies); hTERT-RPE1 cells (ATCC) in Ham's F12/DMEM (Mediatech) supplemented with 10% Fetal Bovine Serum and HCT116 cells (ATCC) were cultured in McCoy's 5A (ATCC) supplemented with 10% Fetal Bovine Serum (Life technologies). For expression of recombinant proteins, HeLa cells were transfected with Lipofectamine 2000 (Life Technologies) 24 h prior to assay. For RNAi, two 48-hour transfections were performed with 20 nM siRNA for Stealth siRNAs or 5 nM for Silencer Select siRNAs using Lipofectamine 2000 according to the manufacturer's protocol. For RPE1 RNAi, 5 nM of siRNA was transfected with Silentfect (BioRad) following manufacturer protocols. IFNγ was from R&D Serotec, JAKi from Calbiochem and Flag-TRAIL from Axxora. His-TRAIL was purified according to standard procedures described in Kim et al., The Journal of biological chemistry 279:40044-40052 (2004).

### PARP13 Knockout Cell Lines

Zinc finger nucleases specific to the PARP13 genomic locus were purchased from Sigma Aldrich and transfected into HeLa Kyoto cells. Monoclonal cell lines (PARP13^{-/-} A/B/C) were generated using serial dilution in 96 well plates, then tested for PARP13 expression via western blot. Three independent monoclonal cell lines lacking PARP13 expression were generated.

### Cloning

GFP-PARP13 has been described previously in Vyas et al., Nature communications 4:2240 (2013). To generate SBP-PARP13, GFP was substituted with streptavidin binding peptide tag using Nhel and BspEl. PARP13^{ΔZnF} and PARP13 RNA binding point mutants were generated using GeneString (Invitrogen) flanked by XhoI/BstXl, which are internal sites in PARP13. PARP13^{ΔZnF} features a deletion from nt228 to nt669.

The psiCHECK2 vector encoding Renilla and Firefly luciferase genes was purchased from Promega. TRAILR4 ORF was purchased from Origene (SC117708). A SalI site was introduced after the TRAILR4 stop codon using a Gene String flanked by PpuMI and ScaI, which are internal sites in TRAILR4 cDNA. The 3′UTR of TRAILR4 was then introduced downstream the Renilla luciferase in psiCHECK2 using SalI/XhoI and NotI digestion. Truncations of TRAILR4 3′UTR were generated by PCR using primers with XhoI/NotI overhangs. psiCHECK2+TRAILR4 3′UTR was used as a template. Fragments were designed based on TRAILR4 3′UTR folding prediction (RNAFold) so as to preserve high-probability folding structures as described in Lorenz et al., Algorithms for molecular biology: AMB 6, 26 (2011).

### Total RNA Purification and Agilent Microarrays

Total RNA purification was performed using Qiagen RNeasy Kit, following manufacturer instructions. Samples were labeled using the Two Color Quick Amp Labeling Kit (Agilent) following manufacturer protocol and hybridized on SurePrint G3 Human Gene Expression v2 8×60 microarray. Microarrays were scanned on SureScan Microarray Scanner (Agilent) and processed with Feature Extractor v10.5. Microarrays have been submitted to GEO, NCBI; accession number GSE56667.

### CLIP

HeLa cells were UV crosslinked at 254 nm with 200 mJ/cm² (Stratagene Stratalinker). For endogenous PARP13 immunoprecipitation, cells were lysed in CLIP Lysis Buffer (1% NP-40, 0.1% SDS, 150 mM NaCl, 1 mM EDTA, 50 mM TRIS (pH7.4), 1 mM DTT), precleared at 16100 g, treated with RNaseA for 10 min at 37 C, immunoprecipitated overnight with PARP13 antibody and washed 2× in CLIP Lysis buffer containing 1M NaCl. Bound RNA was labeled and detected according to Leung et al., Nature structural & molecular biology 18:237-244 (2011). For SBP-PARP13 precipitation, cells were UV crosslinked as described above, lysed with Cell Lysis Buffer (150 mM NaCl, 50 mM HEPES (pH7.4), 1 mM MgCl₂, 0.5% Triton, 1 mM EGTA, 1 mM DTT), precleared at 16100 g, incubated with RNase A for 10 min at 37 C and bound to Streptavidin Sepharose beads (GE Healthcare). RNA bound to SBP-PARP13 was labeled according to Leung et al., Nature structural & molecular biology 18:237-244 (2011) and bound protein eluted with 4 mM biotin.

CLIP qRT-PCR

Cells were UV-crosslinked at 254 nM 200 mV/cm² and lysed in 1% Triton, 125 mM KCl, 1 mM EDTA, 20 mM HEPES pH7.9 under RNase-free conditions. SBP-PARP13 and PARP13 mutants were immunoprecipitated using Streptavidin Sepharose beads. After binding, beads were washed with lysis buffer supplemented with 10 μg/ml tRNA and 250 mM KCl. Proteins were eluted in 4 mM Biotin, treated with Proteinase K, and RNA was purified using Trizol, following manufacturer protocol. Input RNA was collected similarly from total lysate before the immunoprecipitation step. cDNA was prepared from input and bound RNA as described below.

qRT-PCR

cDNA was prepared using ViLo First Strand Kit (Life Technologies) and random primers. 1 μg of total RNA or all CLIP-bound RNA was used per reaction. 100 ng of cDNA was used for each qRT-PCR reaction. Sybr Select reagent (Life Technologies) was used as directed and qRT-PCR was performed on a Roche 480 Light Cycler. Data analysis was performed as previously described in Livak et al., Methods 25:402-408 (2001), using the ΔΔcT method. In all cases ACTB was used as a normalizing control. For gene-specific qRT-PCR primers used in this manuscript refer to table below.

### Dual Luciferase Assays

HeLa cells were transfected with 50 ng of psiCHECK2 constructs in 24-well plates. 48 h post transfection cells were lysed and lysates treated with the Pierce Renilla-Firefly Dual Luciferase Assay Kit as per instructions (Thermo Scientific). Firefly and Renilla luminescence was measured in white 96-well plates in a Tecan Plate Reader (Magenta and Green, 1000 ms each). Renilla luminescence signal was normalized to Firefly signal for each well. For all figures bars represent averages of three individual 24-well plate wells; error bars represent standard deviation.

### Cell Staining and Microscopy

Cells were split onto glass coverslips 16 h before treatment. To induce cytoplasmic stress, cells were incubated with 200 μM Sodium Arsenite for 45 min at 37 C; control cells were left untreated. Unstressed cells were fixed in 4% formaldehyde for 30 min then extracted with Abdil 0.5% Triton for 25 min. Stressed cells were preextracted with HBS containing 0.1% Triton for 1 min, then fixed in 4% Formaldehyde in HBS for 30 min. Blocking and staining was performed as previously described Vyas et al., Nature communications 4:2240 (2013). Fixed cells were blocked in Abdil (4% BSA, 0.1% Triton in PBS), then incubated with antibodies diluted in Abdil for 45 min each.

### Survival Assay

For proliferation assays, 5000 cells were plated in 96 well plates and incubated with recombinant TRAIL the following day for 24 h. Proliferation was analyzed with the Cell Proliferation Kit II (Roche) according to the manufacturer's instructions and survival was calculated by normalizing treated to untreated cells. For apoptosis assays, 40,000 cells were plated in 24 well plates and incubated with recombinant TRAIL for 24 h. Cells were harvested with Trypsin and stained with Annexin V-488 (Biolegend) and propidium-iodide (Sigma) in Annexin binding buffer (10 mM HEPES, 140 mM NaCl, and 2.5 mM CaCl₂, pH 7.4) for 15 min at RT. FACS analysis was performed on a FACScan instrument (BD) and cells negative for Annexin V and propidium iodide considered as alive. For colony forming assays, the indicated numbers of cells were plated in 12 well plates and grown for 7 days in medium with TRAIL changed every second day. Colonies were visualized by staining with 0.02% crystal violet (Sigma) in 50% methanol.

### Electrophoretic Mobility Shift Assays

SBP-PARP13.1 and SBP-PARP13.1^{VYFHR} were purified from HEK293 cells lysed with Cell Lysis Buffer (CLB, 150 mM NaCl, 50 mM HEPES (pH7.4), 1 mM MgCl₂, 0.5% Triton, 1 mM EGTA, 1 mM DTT), precleared at 80000 g, bound to Streptavidin Sepharose beads (GE Healthcare). Beads were washed with CLB containing 1M NaCl, and proteins were eluted with 4 mM Biotin in CLB, then dialyzed overnight in 100 mM KCl, 50 mM TRIS, pH 7.5. Protein concentrations were determined by Coomassie blue stain by comparison to a dilution series of BSA, and by UV spectrophotometry.

Fragment 1 and Fragment E were PCR-amplified, in-vitro transcribed using T7 RNA polymerase, purified and end-labeled with T4 Polynucleotide Kinase and ³²P γATP as previously described in Huan et al., Current protocols in molecular biology Chapter 4, Unit4 15 (2013).

EMSA binding reactions were performed for 1 h at 20 C in 10 mM Tris, pH 7.5, 1 mM EDTA, pH 8, 0.1 M KCl, 0.1 mM DTT, 5% vol/vol Glycerol, 0.01 mg/ml BSA, 0.4 units/μl RNAse inhibitor, 0.1 μg/ml tRNA with 2 nM RNA and decreasing amounts of protein. Reactions were loaded onto 8% TBE Urea gels, and run in 0.5×TBE at room temperature, then exposed to phosphor screen and scanned. To calculate Kd, bands were quantified using ImageJ, fraction bound was calculated, and data was fit to Hill's equation using IGOR Pro.

4—Thiouridine Labeling and mRNA Decay Measurements

Wild type and PARP13^{-/-A} cells were incubated with 200 μM 4-Thioruridine for 2 h, then growth media was changed and cells were collected immediately, and at two hour intervals for 8 h. Total RNA was Trizol extracted at each time point and newly transcribed RNA was biotin-labeled and purified as previously described in Radle et al., Journal of visualized experiments JoVE doi:10.3791-50195 (2013). In brief, newly transcribed RNA was labeled with biotin-HPDP, RNA was repurified, and newly transcribed RNA was separated on streptavidin-coated magnetic beads (Miltenyi). RNA was eluted with 100 mM DTT, and purified using MinElute Cleanup Kit (Qiagen). RT-qPCR was performed as described above. TRAILR4 and GAPDH levels were normalized to ACTB for each sample. Each time point represents an average of three independent experiments; error bars show the standard deviation. Half life was calculated as previously described in Chen et al., Methods in enzymology 448:335-357 (2008). Half-life is an underestimate as expression levels are normalized to ACTB levels, which are also decreasing within this time-course (ACTB half life in HeLa cells is ˜8 h (Leclerc et al., Cancer cell international 2:1 (2002).

### DISC-IP

1×10̂6 wild type or Parp13^{-/-A} cells each were plated in two 10 cm plates for 2 days. Plates were washed once in DMEM (without FCS) and then incubated for 45 min in 2.5 ml DMEM without FCS and with or without 1 μg/ml Flag-TRAIL (Axxora). After addition of 15 ml cold PBS, cells were washed once with 15 ml cold PBS and scraped with a rubber policeman in 1 ml lysis buffer (30 mM Tris/HCl pH7.4, 150 mM NaCl, 5 mM KCl, 10% Glycerol, 2 mM EDTA and protease inhibitors). After addition of 100 μl Triton X-100, lysates were rotated 30 min at 4° C. and harvested by centrifugation (45 min, 4° C., 15000 g). The supernatant was removed, added to 20 μl magnetic Protein-G beads (Invitrogen), washed three times in lysis buffer including Triton X-100 and rotated at 4 C overnight. After five washes in lysis buffer including Triton X-100, beads were heated at 75 C for 10 min in 20 μl loading buffer, subsequently loaded on a gel and blotted for the indicated antibodies.

### Caspase-8 Processing

Wild type and PARP13^{-/-} cells were plated in 6 wells and treated with His-TRAIL for the indicated time periods. Cells were harvested, lysed and analyzed by immunoblot with the indicated antibodies.

### Accession Codes

Microarray data for control and PARP13 knockdowns has been submitted to GEO, NCBI; accession number GSE56667.

TABLE 7
Reagents used in the examples.

| | Catalog number | Company |
|---|---|---|
| siRNAs | | |
| Control | 1027281 AllStar Neg. | Qiagen |
| Scrambled | Control siRNA | |
| siRNA | | |
| PARP13 | GCUCACGGAACUAUGAGCUGAGUUU | Stealth siRNA, Life |
| | (SEQ ID NO: 5)/ | Technologies |
| | AAACUCAGCUCAUAGUUCCGUGAGC | |
| | (SEQ ID NO: 41) | |
| EXOSC5 | S32381 Silencer Select siRNA | Life Technologies |
| TR4 | S16752 Silencer Select siRNA | Life Technologies |
| XRN1 | S29016 Silencer Select siRNA | Life Technologies |
| PARP13.1 | GAAUUUACUUUGCAAAAGAtt | Life Technologies, |
| specific | (SEQ ID NO: 6)/ | Silencer Select |
| | UCUUUUGCAAAGUAAAUUCct | |
| | (SEQ ID NO: 42) | |
| Antibodies | | |
| and | | |
| dilutions | | |
| used | | |
| PARP13 in- | Described in Vyas et al. 2013 | in-house |
| house | | |
| (1:1000) | | |
| PARP13 | GTX120134 | GeneTex |
| (1:1000, IB; | | |
| 1:100, IF) | | |
| TRAILR4 | ab2019 | Abcam |
| (1:500, IB) | | |
| GAPDH | GTX28245 | Genetex |
| (1:2500, IB) | | |
| XRN1 | a300-443A | Bethyl Laboratories |
| (1:1000, IB) | | |
| pSTAT1 | sc-136229 | Santa Cruz Biotechnology |
| (1:1000, IB) | | |
| elF3 | sc-16377 | Santa Cruz Biotechnology |
| (1:5000, IF) | | |
| TRAILR1 | 1139 | Prosci |
| (1:1000, IB) | | |
| TRAILR2 | 2019 | Prosci |
| (1:1000, IB) | | |
| Caspase 8 | 9746 | Cell signaling |
| (1:1000, IB) | | |
| Caspase 8 | 4790 | Cell signaling |
| (1:1000, IB) | | |
| ER Tracker | E34250 | Life Technologies |
| Red (1:1000, | | |
| IF) | | |
| Gene | Forward | Reverse |
| QPCR | | |
| Primers | | |
| TRAILR4 | TTAAGTTCGTCGTCTTCATC | TCTGATCTATGAGATCCTGC |
| | (SEQ ID NO: 7) | (SEQ ID NO: 8) |
| CCL5 | AAGTCTCTAGGTTCTGAGC | TTTTATGGTTGCATTGAGAAC |
| | (SEQ ID NO: 9) | (SEQ ID NO: 10) |
| IFIT2 | ACCATGAGTGAGAACAATAAG | TTAGATAGGCCAGTAGGTTG |
| | (SEQ ID NO: 11) | (SEQ ID NO: 12) |
| OASL | GTACCAGCAGTATGTGAAAG | ATGGTTAGAAGTTCAAGAGC |
| | (SEQ ID NO: 13) | (SEQ ID NO: 14) |
| Renilla | TCCAGATTGTCCGCAACTAC | CTTCTTAGCTCCCTCGACAATAG |
| | (SEQ ID NO: 15) | (SEQ ID NO: 16) |
| EXOSC5 | ACATCCAAGCAAGAAAAGG | CTCAGTGTCTGAGTAGAGC |
| | (SEQ ID NO: 17) | (SEQ ID NO: 18) |
| TRAILR1 | CCACTGAGACTCTGATGC | CAGTTTTGTTGACCCATTTC |
| | (SEQ ID NO: 19) | (SEQ ID NO: 20) |
| TRAILR2 | CACTGAGACTCTGAGACAG | GCTTTAGCCAGCCACCTTTATCTC |
| | (SEQ ID NO: 21) | (SEQ ID NO: 22) |
| TRAILR3 | GTCCAGCAGGATCTCATAG | AAGAAGGTTCATTGTTGGAAG |
| | (SEQ ID NO: 23) | (SEQ ID NO: 24) |
| ACTB | GACGACATGGAGAAAATCTG | ATGATCTGGGTCATCTTCTC |
| | (SEQ ID NO: 25) | (SEQ ID NO: 26) |
| TRAILR4 | GACTTGAGTGGGCTCTTTGT | TATCTGTTACTCAGGGTCTCGT |
| intron-exon 1 | (SEQ ID NO: 27) | (SEQ ID NO: 28) |
| TRAILR4 | CTTGGAGCATTGAGACCCTAAA | GTTTCCTTTGCATGTCCTTCTTC |
| intron-exon 2 | (SEQ ID NO: 29) | (SEQ ID NO: 30) |
| IFIT3 | ATGAGTGAGGTCACCAAG | CCTTGAATAAGTTCCAGGTG |
| | (SEQ ID NO: 31) | (SEQ ID NO: 32) |
| RARRES3 | CTGTAAACAGGTGGAAAAGG | GTATCTCCTAATCGCAAAAGAG |
| | (SEQ ID NO: 33) | (SEQ ID NO: 34) |
| GAPDH | CTTTTGCGTCGCCAG | TTGATGGCAACAATATCCAC |
| | (SEQ ID NO: 35) | (SEQ ID NO: 36) |
| Other | | |
| primers | | |
| Fragment 1 | aaatttaaaTAATACGACTCA | TTTTTTTTTTTTTTTTTTTTgt |
| for in-vitro | CTATAGGGaagaatctcttca | agttagatgtcattaaatgc |
| transcription | ggaaaccagagc | (SEQ ID NO: 38) |
| | (SEQ ID NO: 37) | |
| Fragment E | aaatttaaaTAATACGACTCAC | TTTTTTTTTTTTTTTTTTTTaa |
| for in-vitro | TATAGGGagatgccgcacagcc | tcctgggcatatgtaaacatc |
| transcription | acaatgcttt | (SEQ ID NO: 40) |
| | (SEQ ID NO: 39) | |

### Example 1

### PARP13 Binds to Cellular RNA

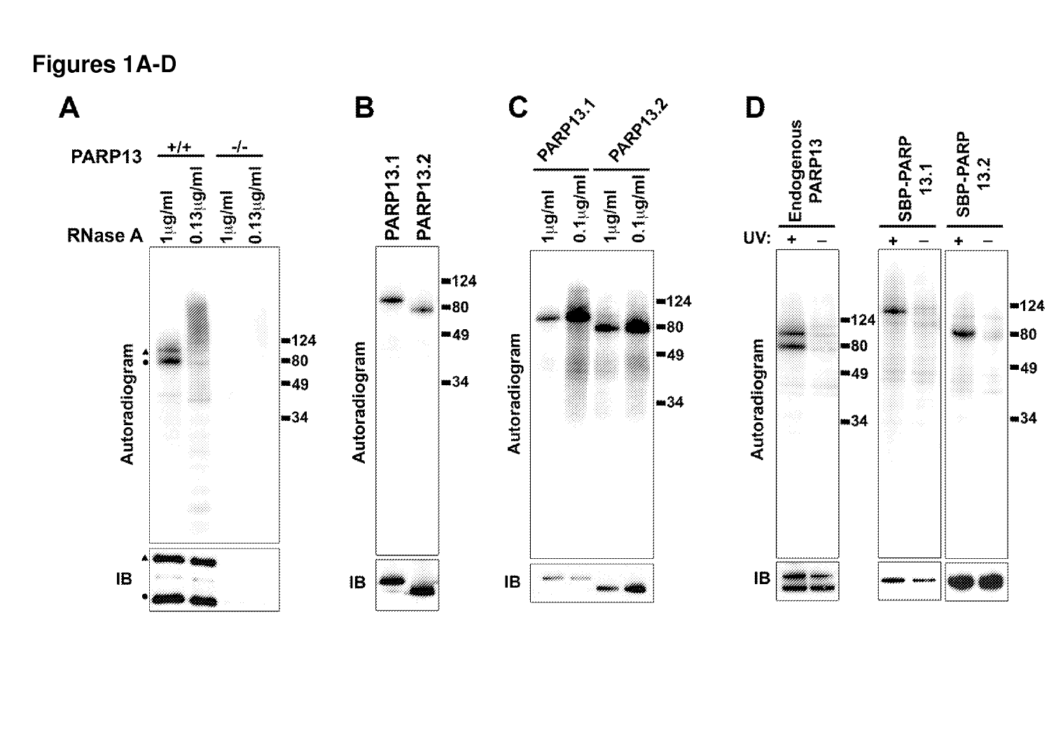

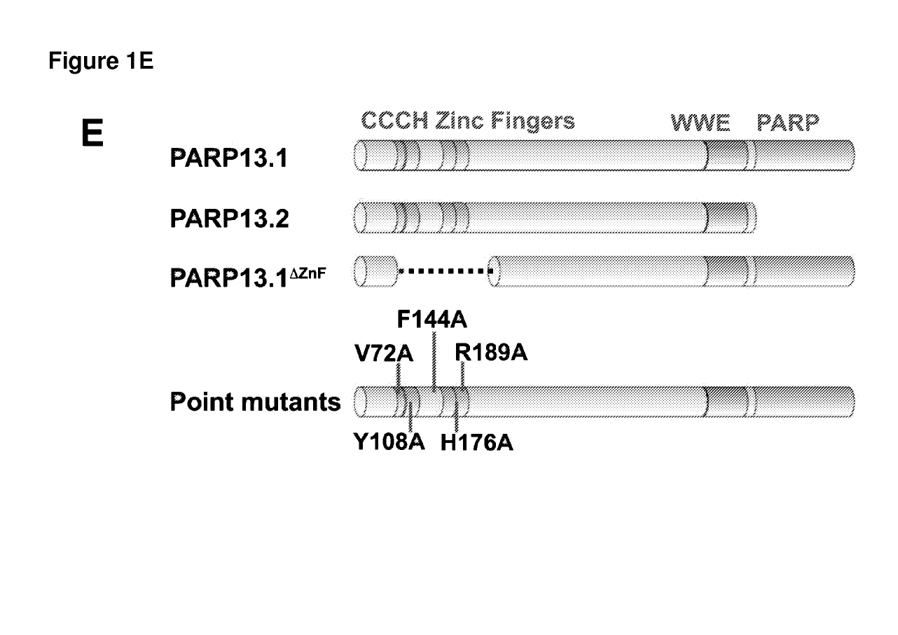

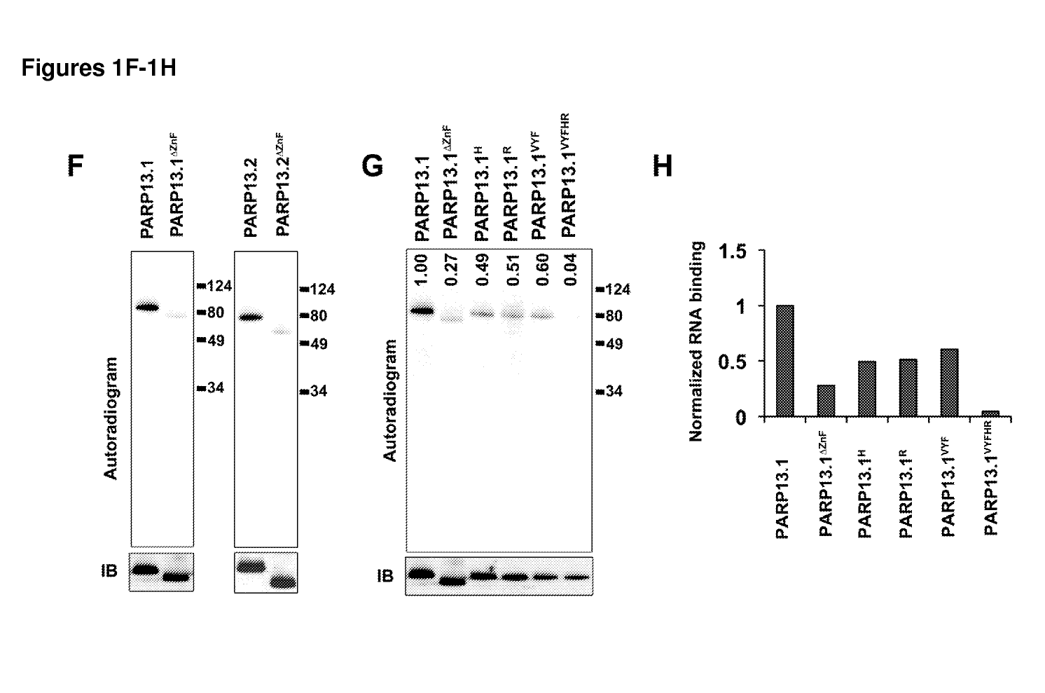

To determine if PARP13 binds to cellular RNA, crosslinking immunopreciptation (CLIP) in HeLa cells using affinity purified PARP13 antibody was performed. A strong signal from bound, crosslinked RNA that collapsed to two major bands at high RNase concentrations was identified (FIG. 1A). The collapsed signal migrated at the molecular weight of PARP13.1 and 13.2, and was PARP13-specific since it was not detected in similar purifications performed in PARP13^{-/-} HeLa cell lines generated using zinc finger nucleases (FIGS. 1A and 9). Since PARP13.1 and PARP13.2 are constitutively expressed in HeLa cells, the binding of cellular RNA to each isoform using N-terminal streptavidin-binding protein (SBP) fusions was compared. SBP-PARP13.1 and SBP-PARP13.2 bound similar amounts of RNA and the signal for both was RNAse sensitive confirming the attached molecules as RNA (FIGS. 1B and 1C). For both the endogenous PARP13 and the SBP precipitations no signal was identified when UV crosslinking was omitted demonstrating the specificity of the reactions (FIG. 1D). To further confirm that binding of RNA to PARP13 is specific and requires the CCCH zinc fingers of PARP13, deletions of these domains from PARP13.1 and PARP13.2 were generated and CLIP (PARP13.1^{ΔZnF} and PARP13.2^{ΔZnF}) was performed (FIGS. 1E and 1F). Deletion of these domains resulted in dramatic reduction of signal.

Structural analysis of the PARP13 RNA binding domain containing four CCCH zinc fingers identified key amino acid residues for viral RNA binding (Chen et al. Nature structural & molecular biology. 19: 430-435,2012). Two cavities, defined by V72, Y108, F144 (Cavity 1) and H176, R189 (Cavity 2) are thought to be important for RNA binding. Each residue of Cavity 1, multiple residues in Cavity 2, and all five residues found in both cavities were mutated to alanine in SBP-PARP13.1 and the mutants assayed for RNA binding using CLIP (FIGS. 1E and 1G, Table 8). Mutation of all five residues to generate PARP13.1^{VYFHR} reduced RNA binding to negligible levels and mutation of all three Cavity 1 residues resulted in a similar decrease in RNA binding as individual mutations of each Cavity 2 residue (FIGS. 1G and 1H).

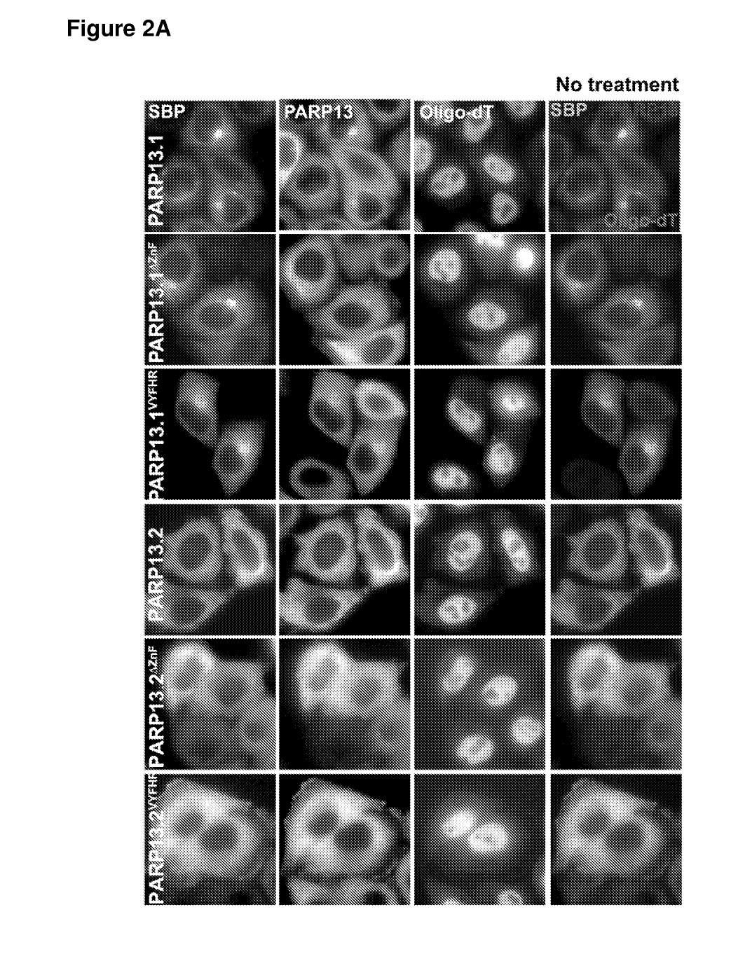

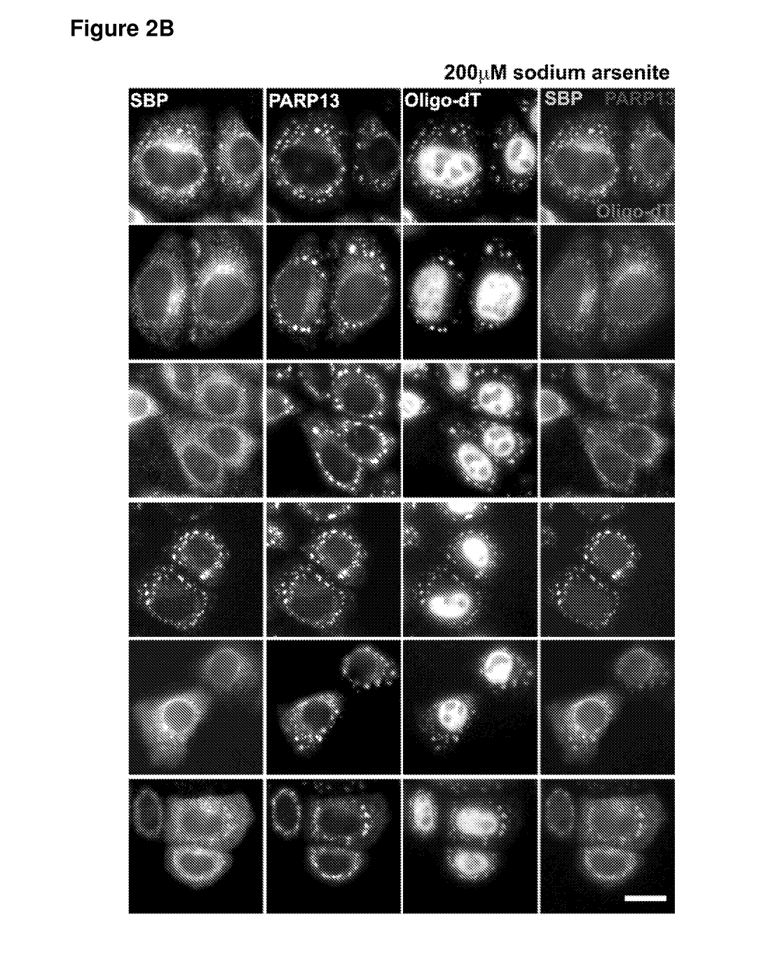

It is possible that the reduction in RNA binding in the mutants was a result of aggregation or mis-localization of the mutant proteins. To test this, the localization of PARP13.1^{ΔZnF} and PARP13.1^{VYFHR} was compared to wild-type protein in HeLa cells. Both mutants exhibited localization patterns similar to PARP13.1 (FIG. 2). Localization of the mutant proteins to stress granules was also examined. It was previously shown that PARP13 is highly enriched in stress granules, structures that are assembled during cytoplasmic stress and contain high concentrations of cellular mRNA (Leung et al. Molecular Cell. 42: 489-499, 2011). PARP13.1 properly localized to stress granules upon sodium arsenite treatment, however both PARP13.1^{ΔZnF} and PARP13.1^{VYFHR} failed to localize to these structures (FIGS. 2 and 10). This defective targeting was even more striking for PARP13.2^{ΔZnF} and PARP13.2^{VYFHR} (FIGS. 2 and 10). These results confirm that the mutants are defective in binding RNA and that this defect affects cellular function. They further suggest that binding to cellular RNA is critical for PARP13 localization to stress granules.

TABLE 8
PARP13 RNA binding mutants

| Mutant | Mutations |
|---|---|
| PARP13.1^{ΔZnF} | Deletion AA 77-223 |
| PARP13.1^{H} | H176A |
| PARP13.1^{R} | R189A |
| PARP13.1^{VYF} | V72A, Y108A, F144A |
| PARP13.1^{VYFHR} | V72A, Y108A, F144A, H176A, R189A |

### Example 2

### PARP13 Regulates the Transcriptome

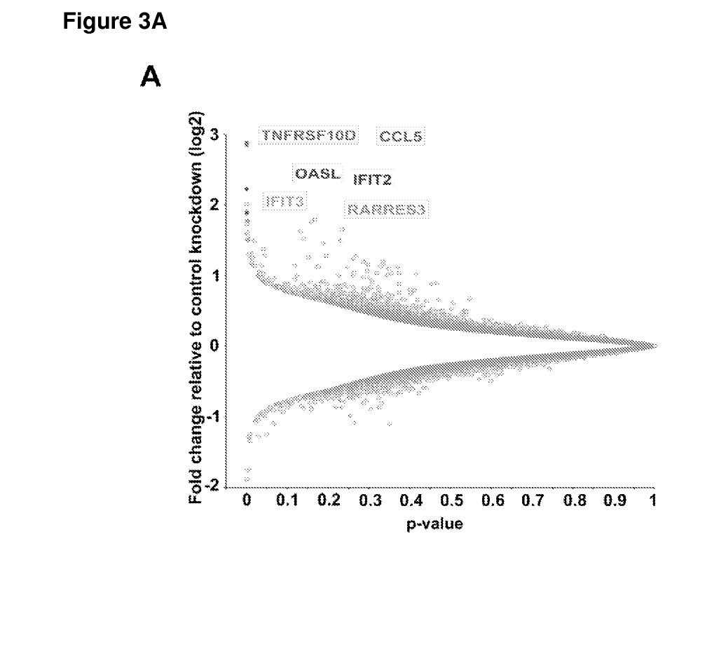

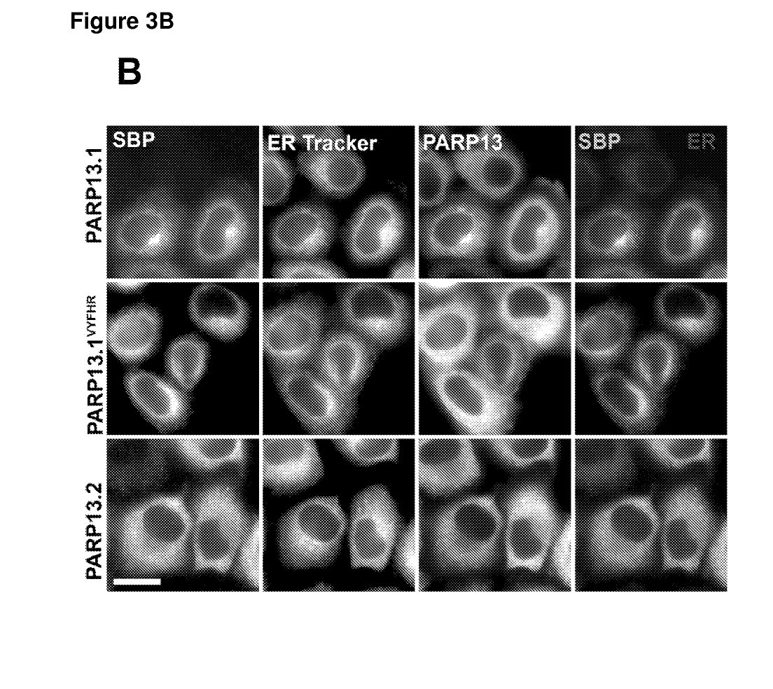

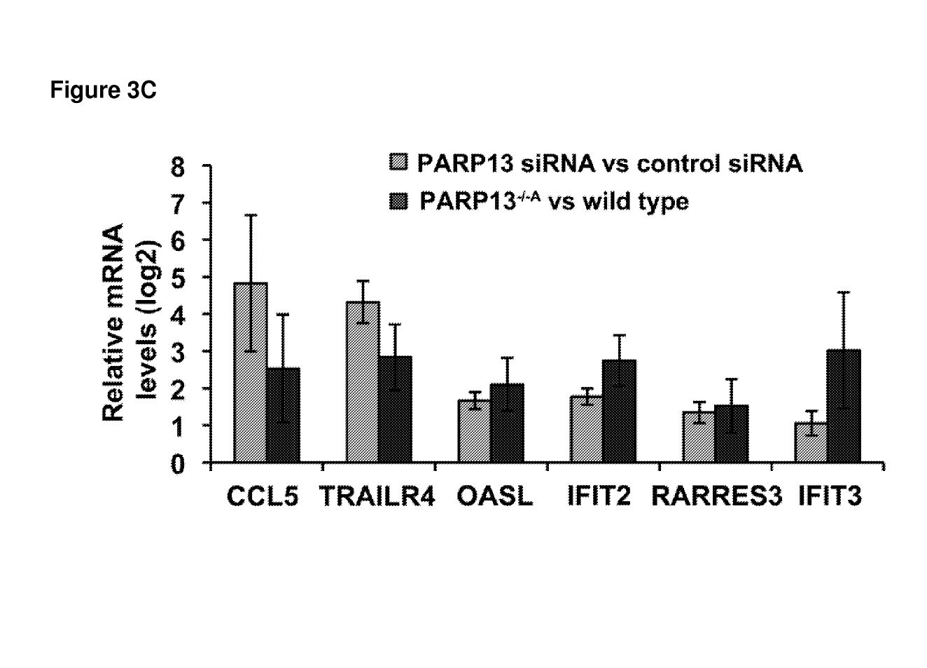

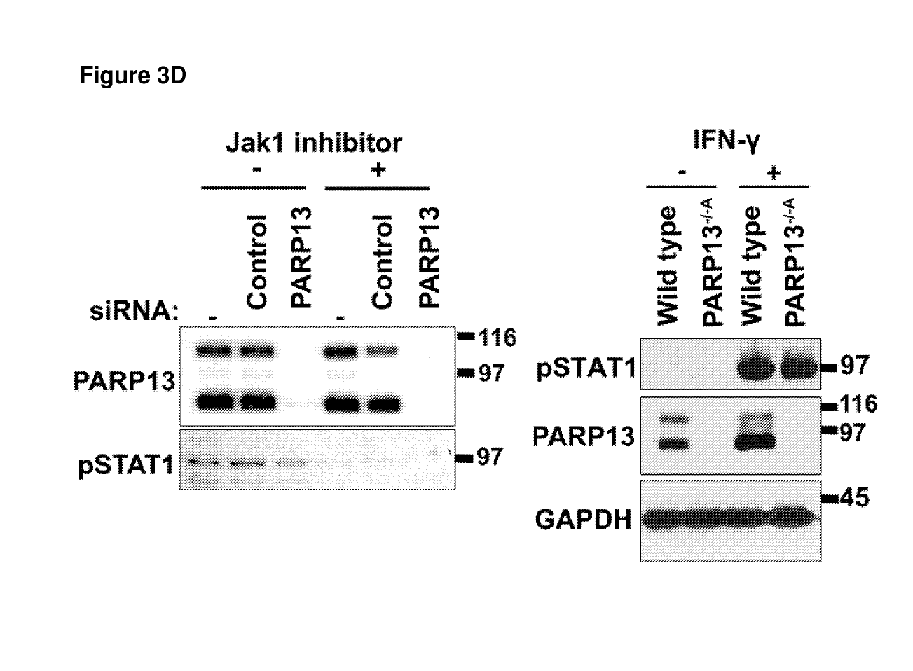

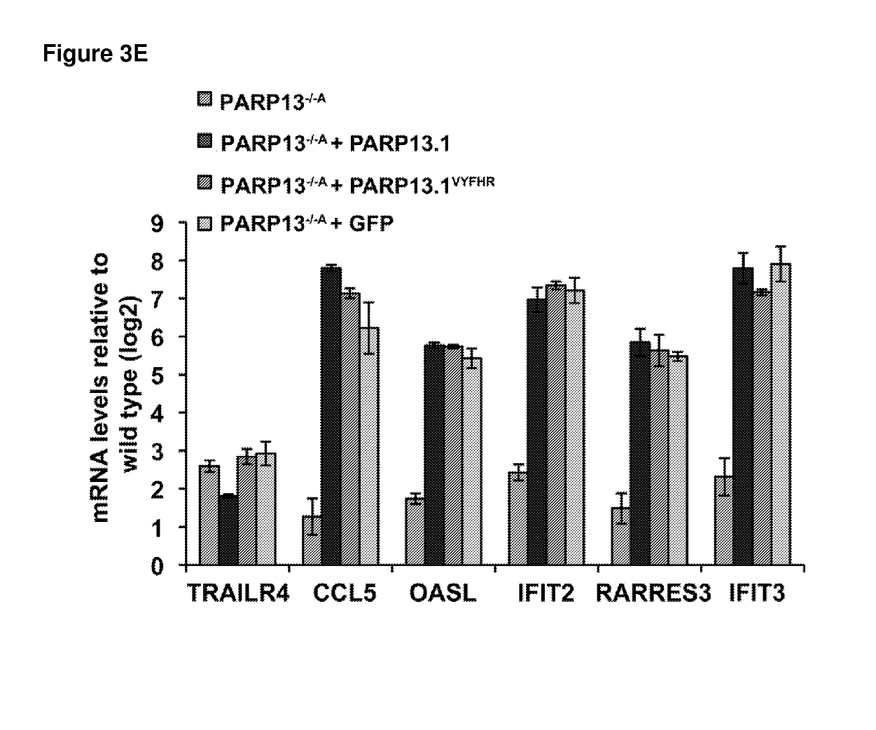

To determine if PARP13 regulates cellular RNA the transcriptome was analyzed in the absence of PARP13. Agilent microarrays were used to compare the relative abundance of transcripts in HeLa cells transfected with control siRNA to cells transfected with PARP13-specific siRNA (FIG. 3A). Depletion of PARP13 resulted in significant misregulation of the transcriptome with 1841 out of a total of 36338 transcripts analyzed showing >0.5 Log 2 fold change (Log 2FC) relative to control knockdowns (1065 upregulated and 776 downregulated transcripts). Of these, 85 transcripts exhibited Log 2FC>1 relative to control siRNAs (66 upregulated and 19 downregulated). In total 73 transcripts passed a significance threshold of p<0.05 (moderated t-statistic with Benjarnini Hochberg adjustment) (Table 9).

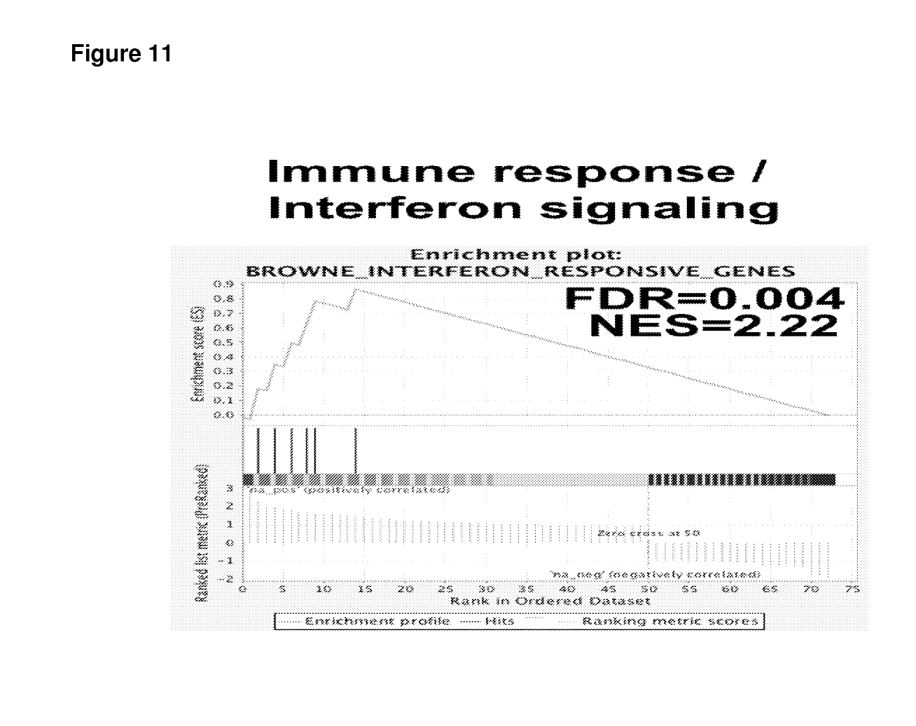

The 50 upregulated transcripts with a p-value <0.05 showed enrichment for genes containing a signal peptide required for targeting of mRNA for translation at the endoplasmic reticulum (ER) (analyzed with the Database for Annotation, Visualization and Integrated Discovery (DAVID) (Huang et al., Nature protocols 4:44-57 (2009)), Enrichment Score 3.4, p-value<0.0001), suggesting that PARP13 could regulate transcripts at the ER. The membranous perinuclear localization observed for PARP13.1 (FIG. 2) and the previously reported membrane targeting of this protein (Charron et al., Proceedings of the national Academy of Sciences of the United States of America 110:11085-11090 (2013)) suggested a potential enrichment at the ER. Therefore, costained exogenously expressed PARP13 isoforms was costained with the ER marker ER Tracker, and colocalization was observed with PARP13.1 but not PARP13.2. This localization is independent of RNA binding since PARP13.1^{VYFHR} localized very similarly to the wild type protein (FIG. 3B). Targeting of PARP13 to the ER may therefore be one mechanism of regulating its function and its RNA target specificity. Gene Set Enrichment Analysis (Subramanian e al., Proceedings of the national Academy of Sciences of the United States of America 102:15545-15550 (2005)) of the same genes identified enrichment for members of the interferon immune response pathway (p-value<0.0001, Normalized Enrichment Score=2.22) (FIG. 11).

To verify our results, 6 of the top 10 most upregulated transcripts were analyzed using quantitative real-time reverse-transcription PCR (qRT-PCR) in both PARP13 knockdowns and PARP13^{-/-} cells. All 6 transcripts were upregulated relative to controls upon PARP13 depletion (FIG. 3C). With the exception of TRAILR4/TNFRSF10D, each of these genes encodes an immune response gene and is a member of the interferon-stimulated genes (ISGs), activated in response to interferon signaling. The upregulation of the five ISGs appears to be specific and is not the result of a general activation of the interferon response since JAK-STAT signaling was not increased in the knockdown or knockout cells (FIG. 3D) and since other canonical ISGs (IRFs, TRIMS, IFITMs) were not upregulated in our transcriptome analysis. Interestingly, like PARP13, the upregulated ISGs (OASL, IFIT2, IFIT3) function in inhibition of viral replication and translation (Schoggins et al., Current opinion in virology 1:519-525 (2011) suggesting that their upregulation could be a compensatory mechanism for PARP13 depletion.

To identify the direct targets of PARP13 regulation among the 6 highly upregulated transcripts, the expression levels in PARP13 cells relative to PARP13 cells expressing wild type PARP13 or PARP13 RNA-binding mutant were compared. While both PARP13.1 and PARP13.2 are constitutively expressed in HeLa cells, PARP13.2 expression increases during viral infection in an interferon dependent manner, whereas PARP13.1 expression does not (Hayakawa et al., Nature immunology 12:37-44 (2011). Therefore to exclude interferon-related effects the experiments were focused on PARP13.1. Direct targets of PARP13 binding and regulation would in theory decrease upon PARP13.1 but not PARP13^{VYFHR} expression in PARP13^{-/-} cells. TRAILR4 mRNA clearly behaved in this manner with a 40% decrease in transcript levels upon PARP13.1 expression and no change upon PARP13.1^{VYFHR} expression (FIG. 3E).

TABLE 9
Number of differentially expressed
transcripts upon PARP13.1 depletion

| | Number of | % of | Up- | Down- |
|---|---|---|---|---|
| Cut-off | genes | total | regulated | regulated |
| Log2FC > 0.5 | 1841 | 5.066 | 1065 | 776 |
| Log2FC > 1 | 85 | 0.234 | 66 | 19 |
| p-value < 0.1 | 134 | 0.369 | 84 | 50 |
| p-value < 0.05 | 73 | 0.201 | 50 | 23 |
| Log2FC > 1/ | 49 | 0.135 | 34 | 15 |
| p-value < 0.05 | | | | |

### Example 3

### PARP13 Represses TRAILR4 mRNA and Protein Expression

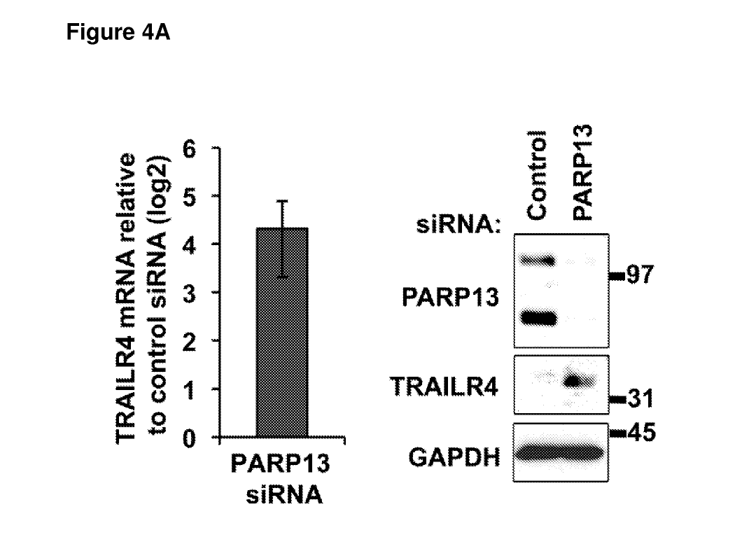

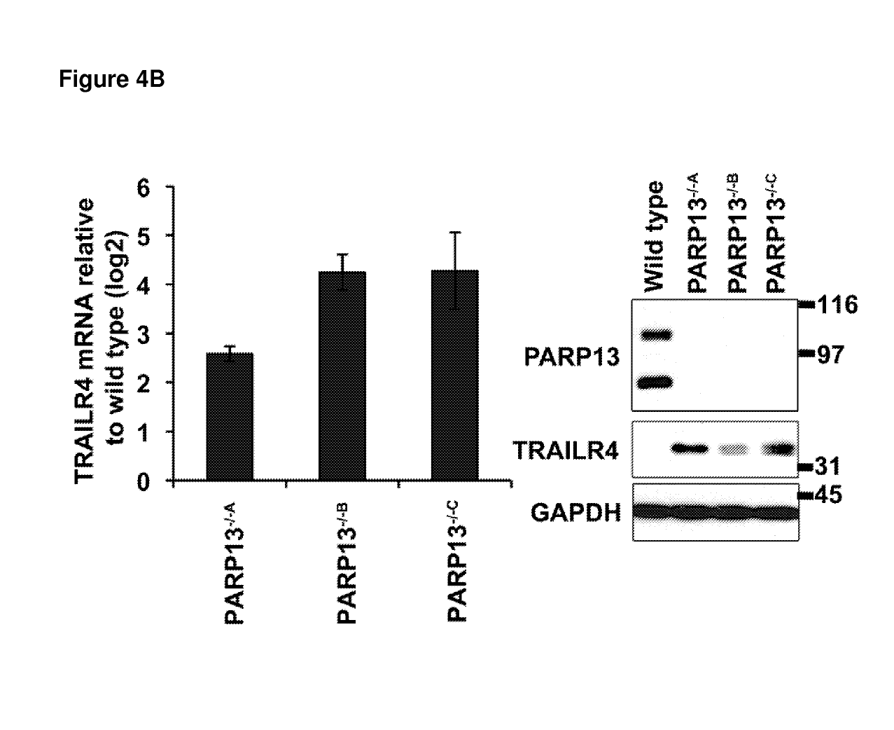

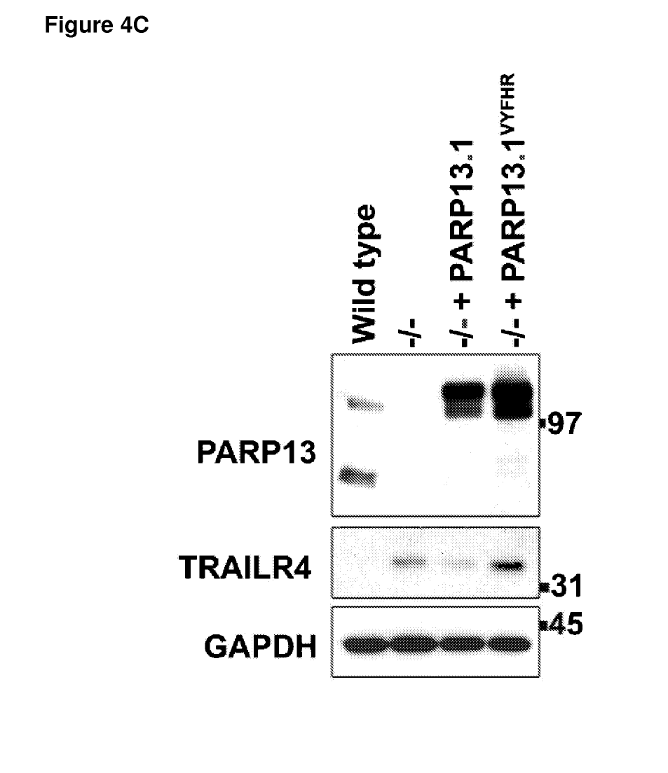

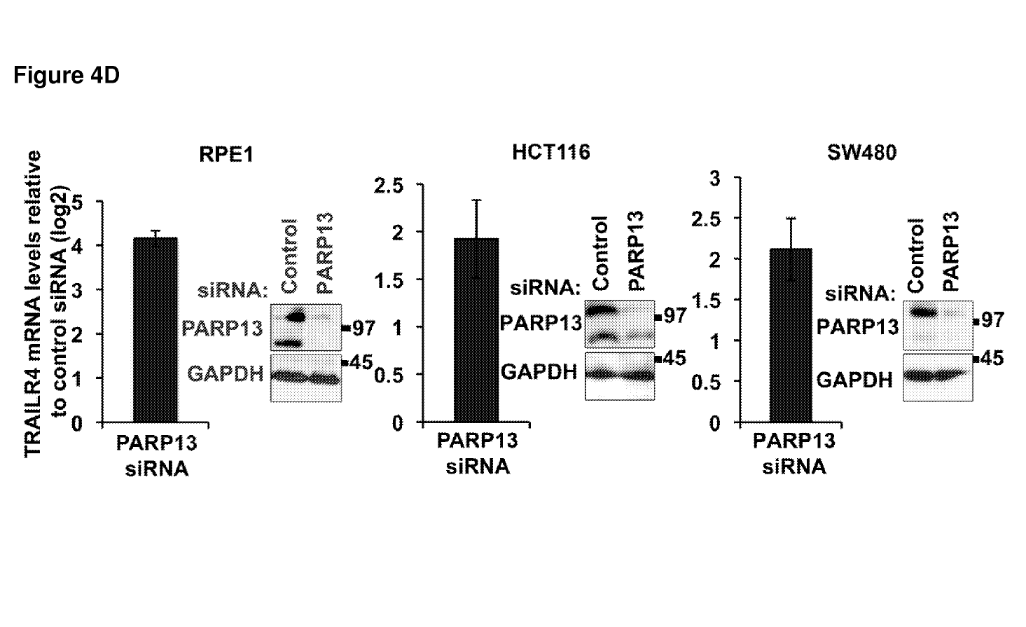

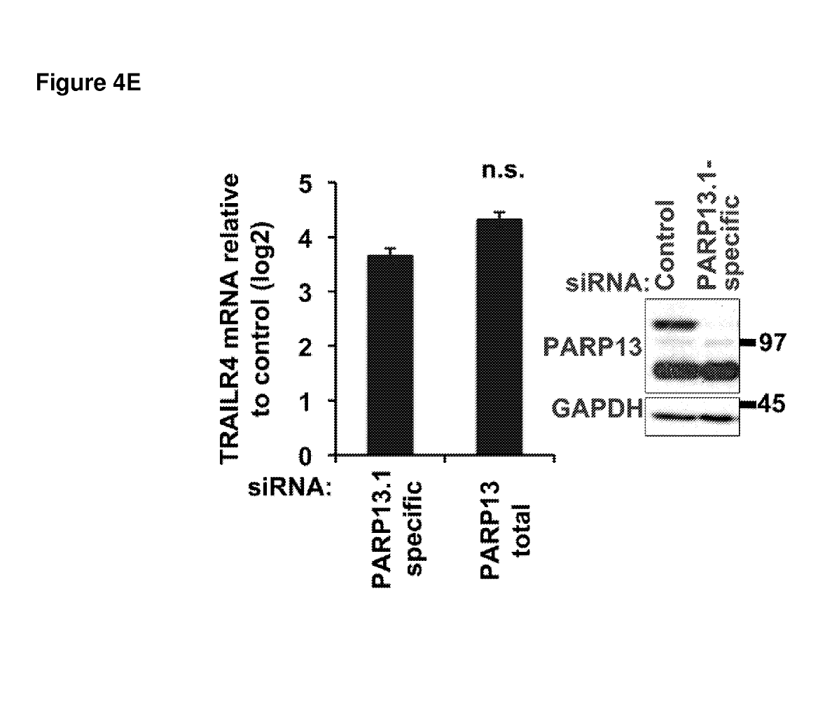

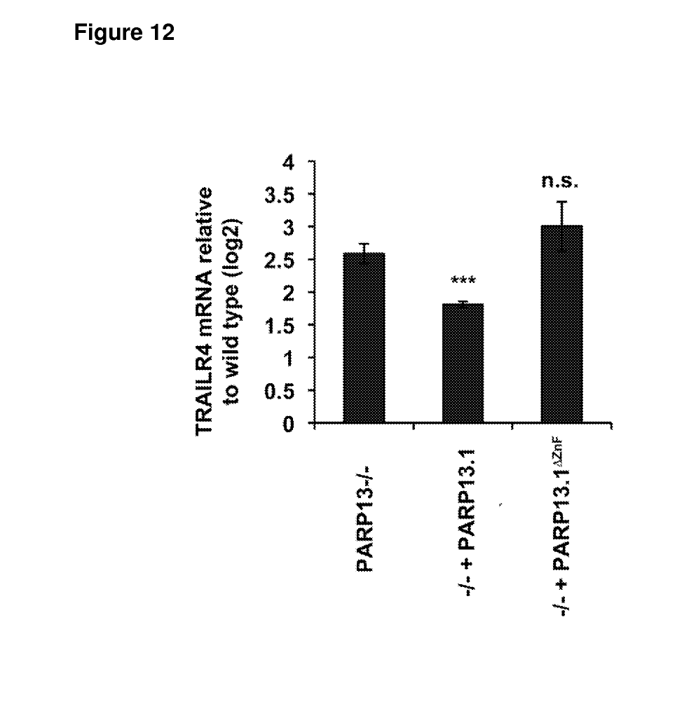

Due to its biological importance and the clinical interest in TRAIL the role of PARP13 in the regulation of TRAILR4 expression and how that regulation might impact TRAIL signaling and apoptosis was examined. Upregulation of TRAILR4 mRNA in PARP13-depleted HeLa cells had a direct effect on TRAILR4 protein expression: TRAILR4 protein levels, barely detectable in wild type HeLa cells, increased in PARP13 knockdown cells and in all three independently isolated PARP13^{-/-} cell lines (FIGS. 4A and 4B). In addition, consistent with the results identifying TRAILR4 as a direct target of PARP13 regulation (FIGS. 3E and 12), expression of PARP13.1, but not PARP13.1^{VYFHR}, in PARP13^{-/-A} cells was sufficient to reduce TRAILR4 protein expression (FIG. 4C). PARP13 repression of TRAILR4 mRNA represents a general mechanism of TRAILR4 regulation in multiple human cell types. In all cell lines tested, including primary cells such as Tert immortalized Retinal Primary Epithelial (RPE1) cells and transformed cells such as human colon HCT116, human colon adenocarcinoma SW480 and HeLa cells, TRAILR4 mRNA levels increased upon PARP13 depletion identifying suppression of TRAILR4 expression as an important physiological function of PARP13 (FIG. 4D). Under physiological conditions, the primary isoform of PARP13 that regulates TRAILR4 is PARP13.1 since specific knockdown of PARP13.1 in HeLa cells increased TRAILR4 mRNA to levels similar to those obtained upon total PARP13 depletion (FIG. 4E).

### Example 4

### PARP13 Inhibits TRAILR4 Post-Transcriptionally Via its 3′UTR

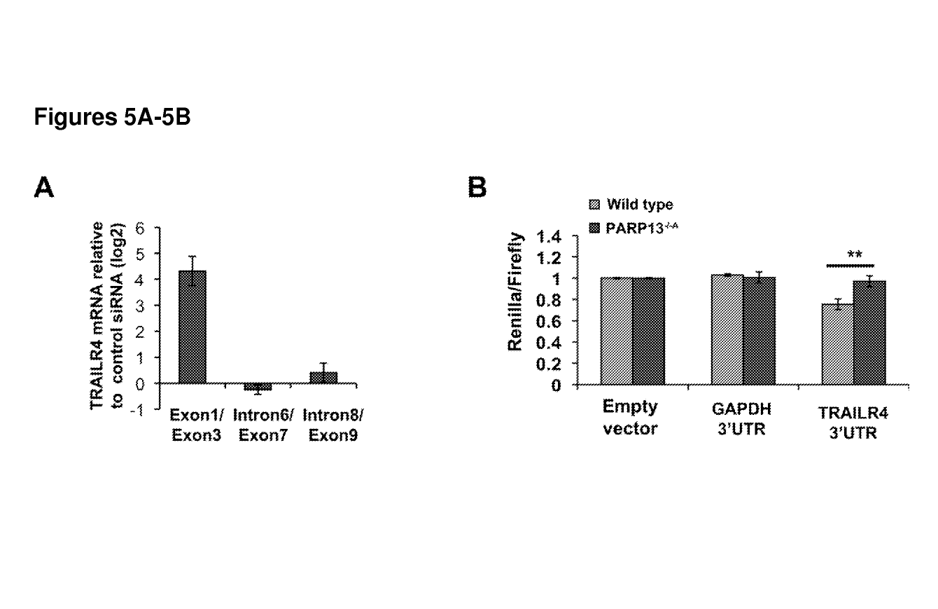

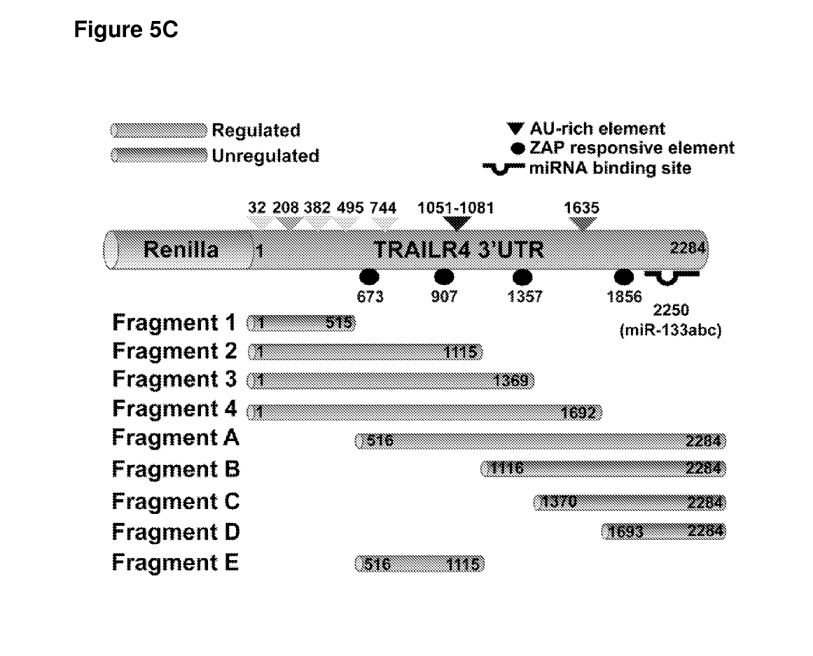

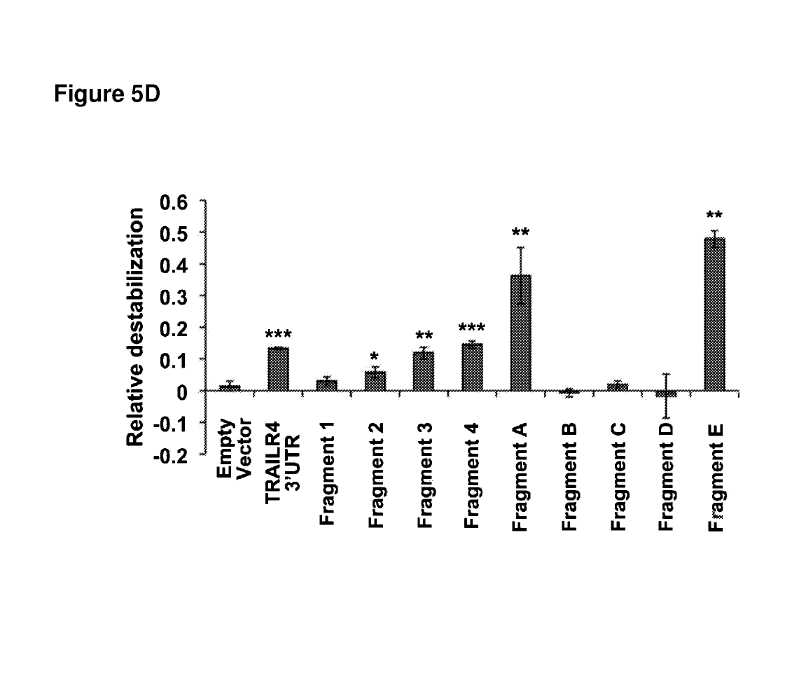

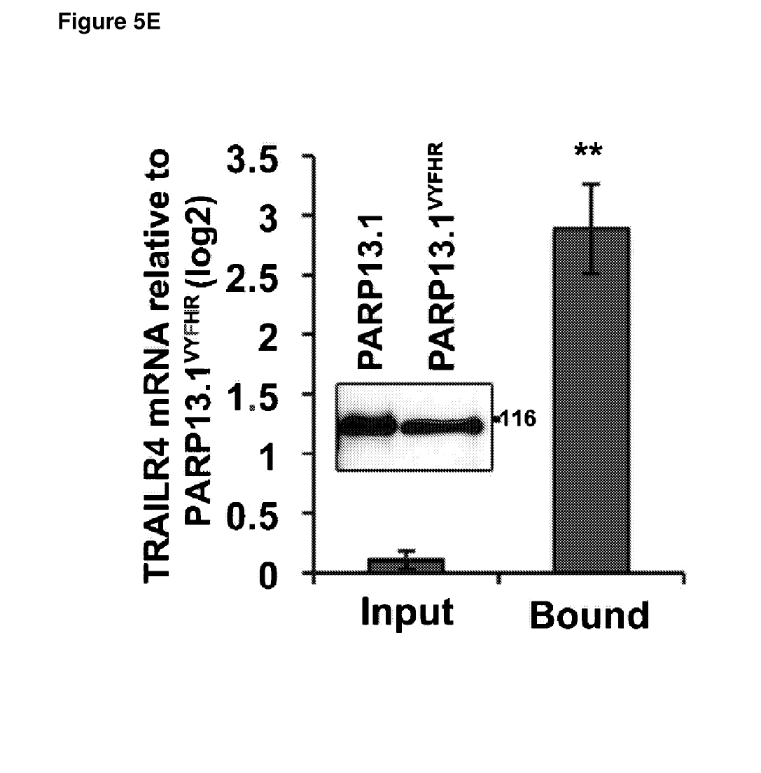

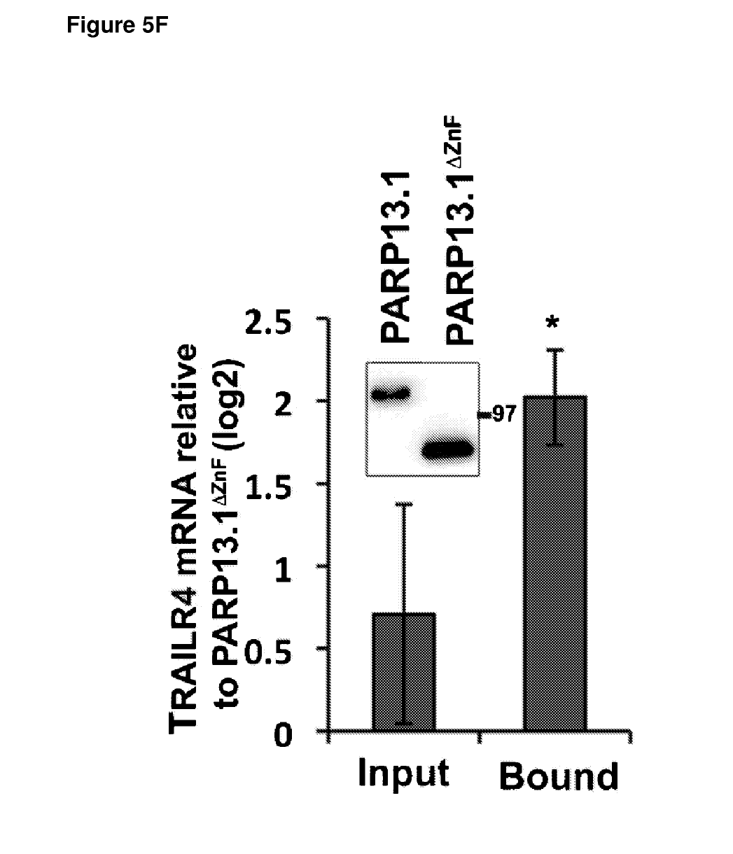

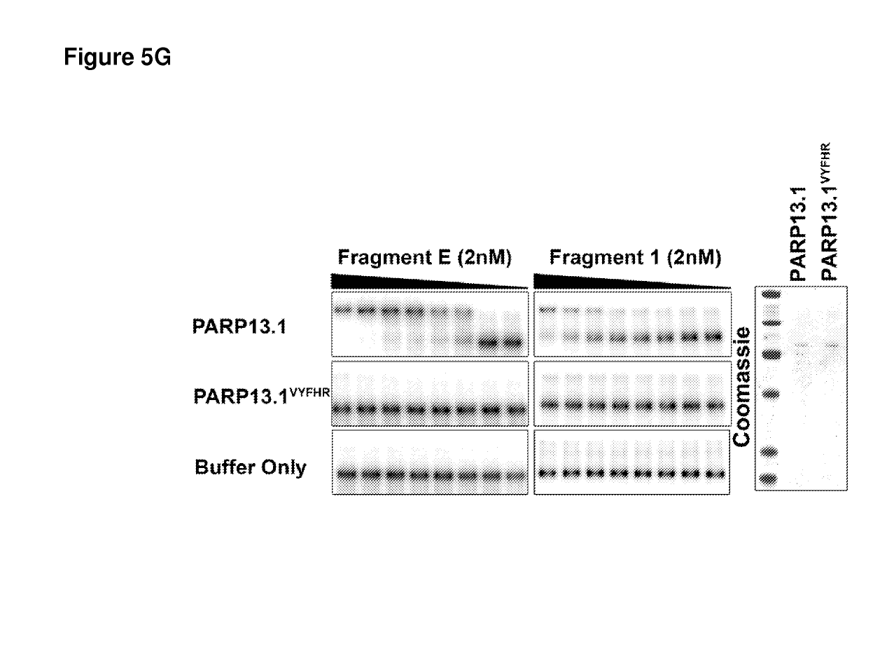

The PARP13.1 and PARP13.1^{VYFHR} rescue assays performed in PARP13^{-/-} cells suggest that TRAILR4 regulation by PARP13 is posttranscriptional and requires RNA binding to PARP13 (FIGS. 3E and 12). Posttranscriptional regulation was confirmed by analyzing the upregulated TRAILR4 transcripts via qRT-PCR with primers that overlap intron-exon boundaries to identify unspliced pre-mRNA, and primers that overlap exon-exon boundaries, to identify mature transcripts. PARP13 knockdown resulted in increased amounts of mature TRAILR4 mRNA, but had no effect on the amount of pre-mRNA, suggesting that TRAILR4 transcription is not altered upon PARP13 depletion and that regulation of TRAILR4 by PARP13 is posttranscriptional (FIG. 5A). Since posttranscriptional regulation of mRNA often occurs via the 3′ untranslated region (3′UTR) reporter constructs were designed containing the 3′UTR of TRAILR4 or GAPDH (as negative control) fused to Renilla luciferase in the psiCHECK2 vector. This vector also encodes Firefly luciferase as a transfection control. Renilla-TRAILR4 3′UTR expression was decreased ˜20% in HeLa cells relative to PARP13^{-/-} cells whereas no significant difference in Renilla or Renilla-GAPDH 3′UTR expression was detected between the two cell lines (FIG. 5B). Together these results suggest that PARP13 destabilizes TRAILR4 posttranscriptionally via its 3′UTR.

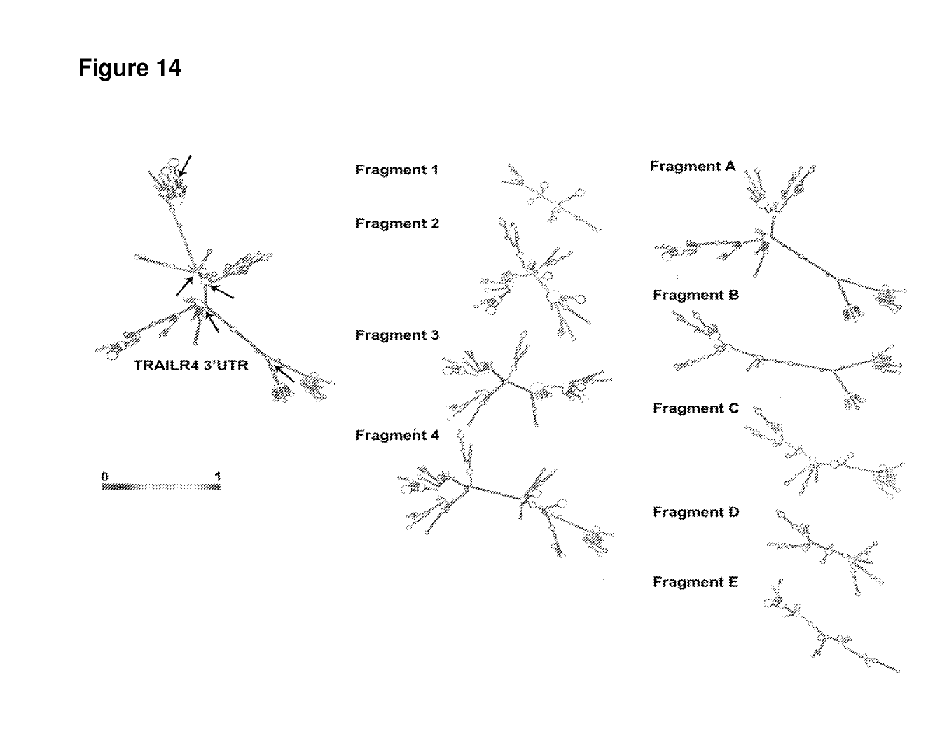

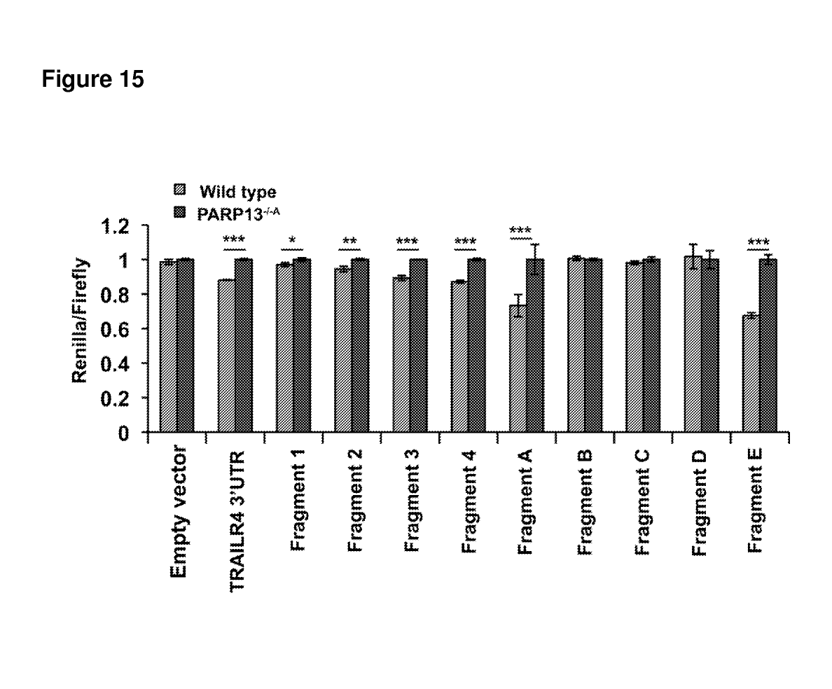

Computational analysis of the TRAILR4 3′UTR identified 7 putative AU rich elements (ARE) known to destabilize RNA (Schoenberg et al., Nature reviews. Genetics 13:246-259 (2012); Gruber et al., Nucleic acids research 39:D66-69 (2011)), one conserved miRNA binding site that is muscle specific (miR-133abc) (Luo et al., Journal of genetics and genomics=Yi chuan xue bao 40:107-116 (2013); Lorenz et al., Algorithms for molecular biology:AMB 6:26 (2011)) and 4 short and poorly characterized ZAP responsive elements (ZRE) predicted by SELEX to mediate PARP13 recognition of RNA targets (Huang et al., Protein & cell 1:752-759 (2010)) (FIGS. 5C and 13). To identify the key PARP13 dependent regulatory sequences in the TRAILR4 3′UTR, truncations of the TRAILR4 3′UTR were designed and fused to Renilla luciferase in the psiCHECK2 vector (FIG. 5C). Fragments were designed based on a secondary structure prediction of the TRAILR4 3′UTR to avoid disturbing high-probability RNA folds (Lorenz et al., Algorithms for molecular biology:AMB 6, 26 (2011)) (FIG. 14). The relative PARP13-dependent destabilization of each fragment was determined by subtracting expression in wild type cells from expression in PARP13^{-/-} cells (FIGS. 5D and 15). This analysis identified nucleotides 516-1115 of the 3′UTR as necessary for PARP13 regulation. Fusion of nucleotides 516-1115 (Fragment E) to Renilla resulted in destabilization of the construct in wild type cells, confirming that this sequence contains the relevant signal for PARP13-dependent repression (FIG. 5D). This fragment includes 2 ZREs and 2 AREs, including one that contains multiple overlapping ARE sequences suggesting that PARP13 regulation of TRAILR4 mRNA might require ARE and/or ZRE recognition. The analysis also suggests that TRAILR4 regulation is likely miRNA independent since no predicted miRNA binding sites are found in the TRAILR4 regulatory sequence.

### Example 5

### PARP13 Binds TRAILR4 mRNA

To determine if PARP13 regulation of TRAILR4 occurs via direct binding to TRAILR4 mRNA, CLIP qRT-PCR in cells expressing SBP-PARP13.1, SBP-PARP13.1^{VYFHR} or PARP13.1^{ΔZnF} and electrophoretic mobility shift assays (EMSA) using purified SBP-PARP13.1 or SBP-PARP13.1^{VYFHR} and ³²P labeled Fragment E or Fragment 1 as control were performed. CLIP qRT-PCR analysis identified significant enrichment of TRAILR4 mRNA in wild type PARP13.1 precipitations relative to PARP13.1^{VYFHR} or PARP13.1^{ΔZnF} confirming a direct and specific binding interaction between TRAILR4 mRNA and PARP13 in vivo (FIGS. 5E and 5F). These results were confirmed in vitro by EMSA assays where SBP-PARP13.1 bound to Fragment E with high affinity (Kd=123 nM) and to Fragment 1 with lower affinity (Kd=508 nM). PARP13.1^{VYFHR} failed to bind either fragment (FIG. 5G). These experiments demonstrate the specificity of TRAILR4 mRNA binding to PARP13 and show that the regulatory region of the TRAILR4 3′UTR binds directly to PARP13 with good selectivity (FIG. 5G).

### Example 6

### PARP13 Destabilization of TRAILR4 mRNA is Exosome Dependent

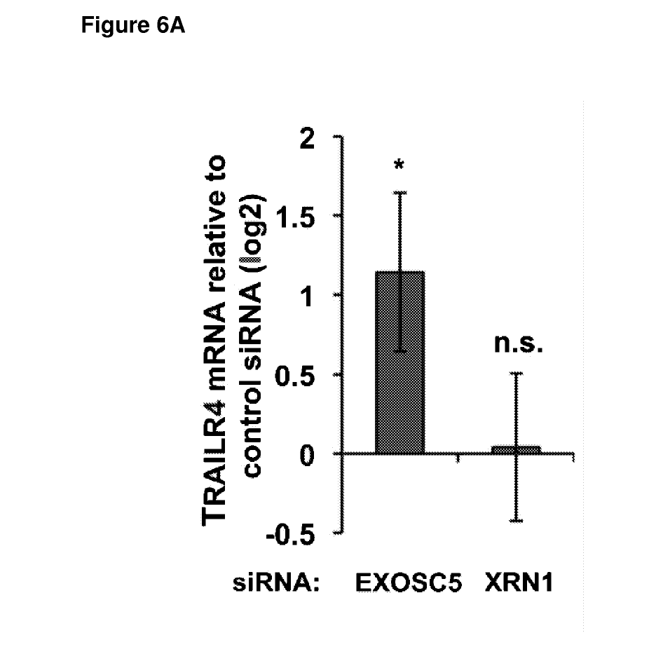

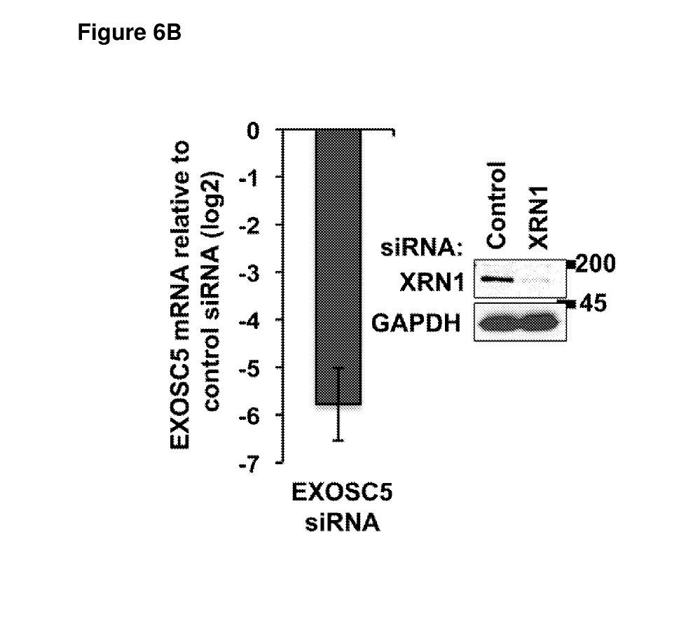

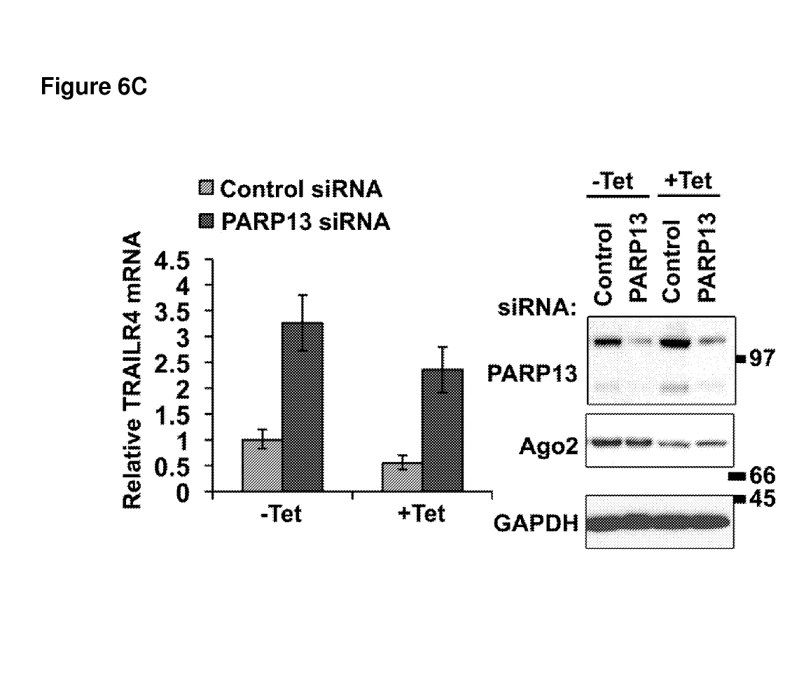

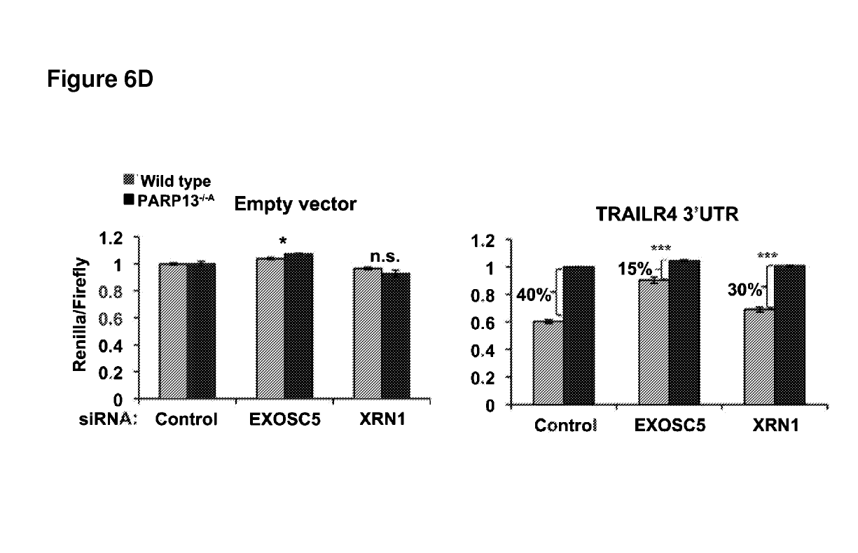

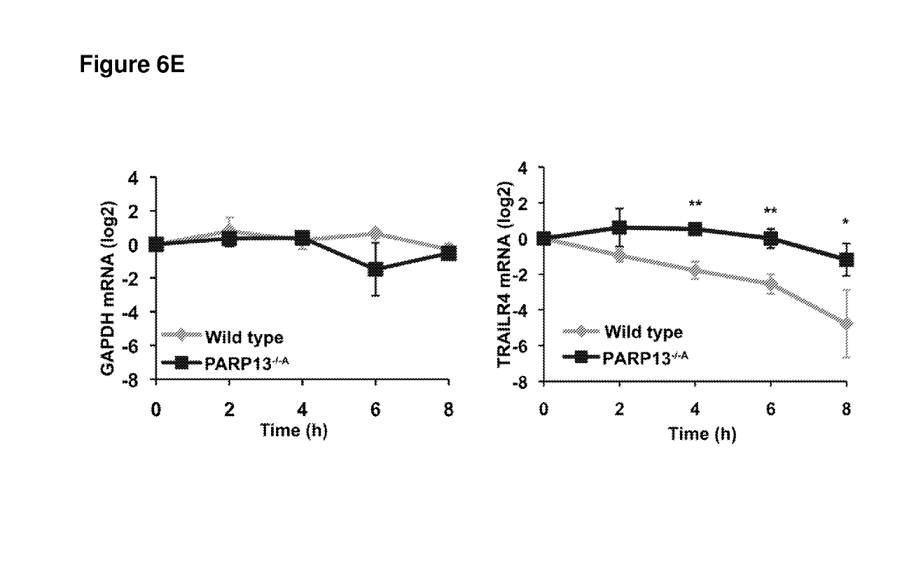

PARP13 regulates viral RNA stability via XRN1-dependent 5′-3′ decay, and exosome-dependent 3′-5′ decay (Zhu et al., Proceedings of the National Academy of Sciences of the United States of America 108:15834-15839 (2011)). PARP13 can also bind to and modulate Argonaute (Ago) activity, critical for miRNA dependent posttranscriptional regulation of mRNA stability (Leung et al., Molecular cell 42:489-499 (2011). To determine if TRAILR4 mRNA stability is regulated through any of these pathways, TRAILR4 mRNA levels were examined upon knockdown of Ago2, XRN1 or EXOSC5, an exosome complex component shown to bind PARP13 (Guo et al., Proceedings of the National Academy of Sciences of the United States of America 104:151-156 (2007). Knockdown of EXOSC5, verified by qRT-PCR (antibodies were non-reactive), resulted in stabilization of TRAILR4 mRNA in HeLa cells suggesting that exosome function is necessary for regulation of TRAILR4 mRNA (FIGS. 6A and 6B). In contrast, neither XRN1 knockdown in HeLa cells nor depletion of Ago2 in HEK293 cells using tetracycline-inducible Ago2 shRNA (Schmitter et al., Nucleic acids research 34:4801-4815 (2006) resulted in obvious TRAILR4 mRNA stabilization (FIGS. 6A-6C). PARP13 depletion in the Ago2 shRNA inducible cell lines resulted in similar levels of TRAILR4 upregulation regardless of Ago2 depletion, further suggesting that Ago2 function is not necessary for TRAILR4 regulation by PARP13 (FIG. 6C).

To determine if exosome or XRN1 activity is required for PARP13 dependent destabilization of TRAILR4 mRNA, the expression of psiCHECK2 reporter constructs encoding Renilla and Renilla-TRAILR4 3′UTR in wild type and PARP13^{-/-} cells transfected with control, EXOSC5 or XRN1 siRNA were examined (FIG. 6D). Relative PARP13-dependent destabilization was then calculated by subtracting the Renilla/Firefly luciferase signal obtained from wild type cells from the signal obtained from PARP13^{-/-} cells (FIG. 6D). Knockdown of EXOSC5 resulted in a pronounced defect in the ability of PARP13 to repress TRAILR4 3′UTR with a 15% relative destabilization of the construct compared to 40% in control knockdowns. Knockdown of XRN1 resulted in milder but potentially relevant defects with a 30% relative destabilization of the Renilla-TRAILR4 3′UTR compared to 40% in controls consistent with the observation that exosome activity was the primary pathway regulating endogenous TRAILR4 mRNA (FIG. 6A). It was thus concluded that PARP13 requires exosome activity, and potentially XRN1, to destabilize TRAILR4 mRNA (and likely other PARP13 targets).

### Example 7

### PARP13 Decreases TRAILR4 mRNA Half-Life

Since the exosome complex is a key regulator of mRNA decay, the TRAILR4 mRNA decay rate in PARP13^{-/-} and wild type cells was examined. Newly transcribed RNA was pulse-labeled with 4-thiouridine and labeled transcripts purified at specific time points after 4-thiouridine removal. qRT-PCR was then performed on the purified transcripts to quantitate amounts of TRAILR4 mRNA and GAPDH mRNA. ACTB mRNA was used to normalize inputs. TRAILR4 mRNA decay rates were significantly higher in wild type cells (t_{1/2}=1.5 h) than in PARP13^{-/-} cells (t_{1/2}=13 h) whereas GAPDH decay rates were similar in both cell lines (FIG. 6E). Together the data is consistent with a model in which PARP13 facilitates efficient degradation of TRAILR4 mRNA via the activity of the exosome complex (FIGS. 6D and 6E) and suggest that PARP13 functions as a novel RNA binding protein that regulates cellular RNA stability by binding to the 3′UTR.

### Example 8

### PARP13 Depletion Inhibits TRAIL-Induced Apoptosis

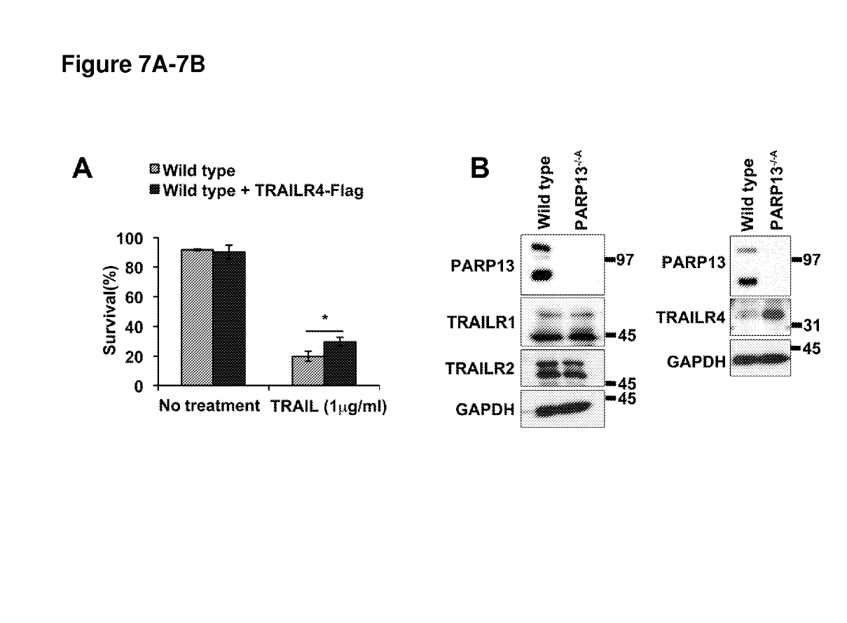

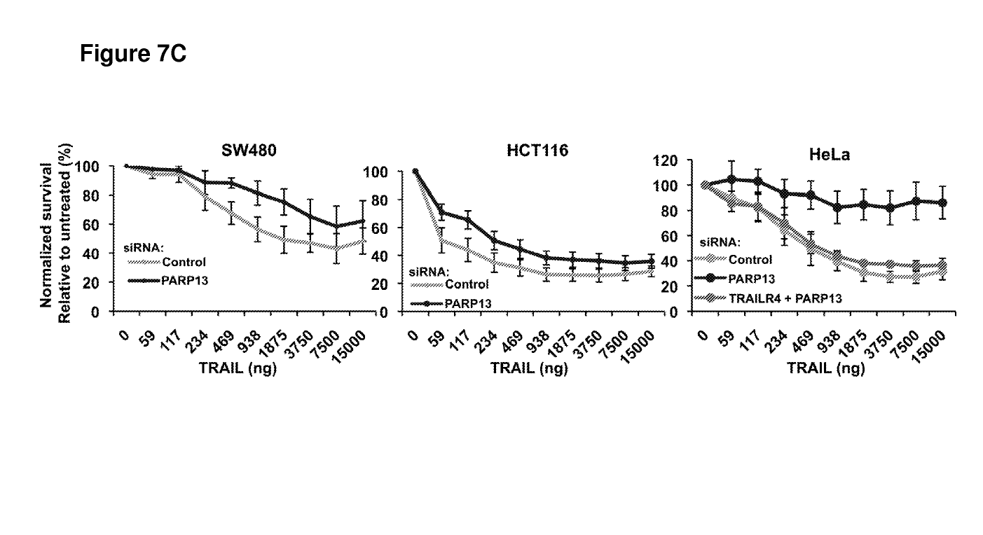

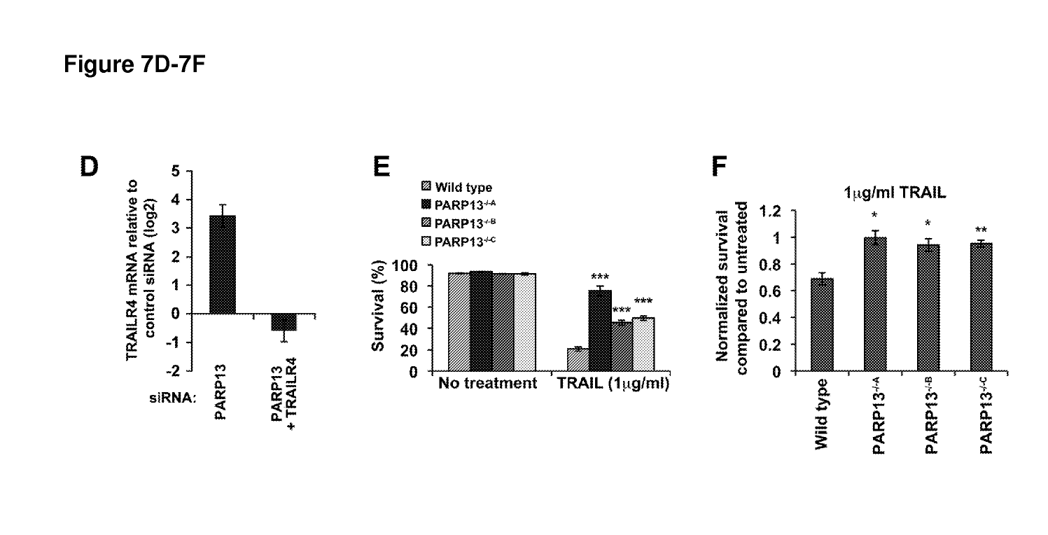

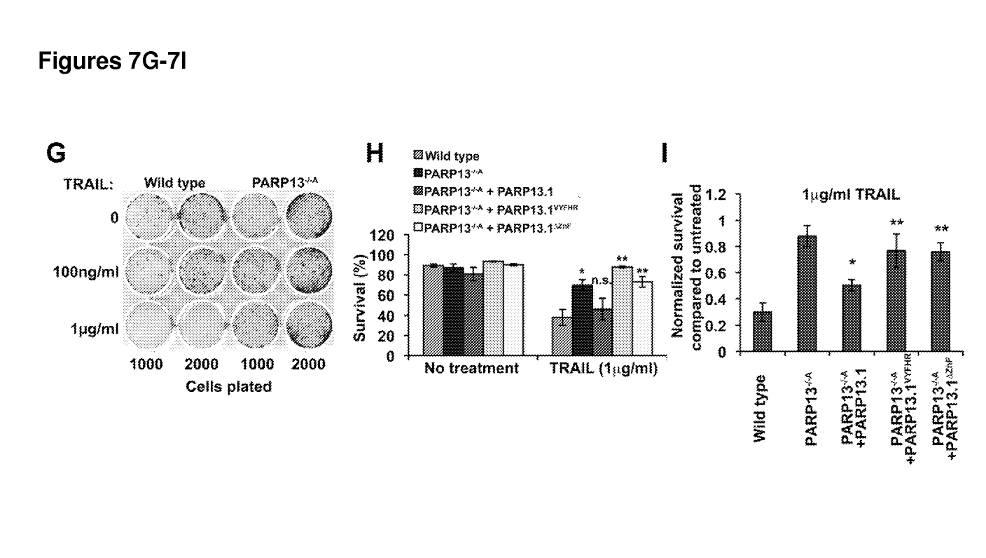

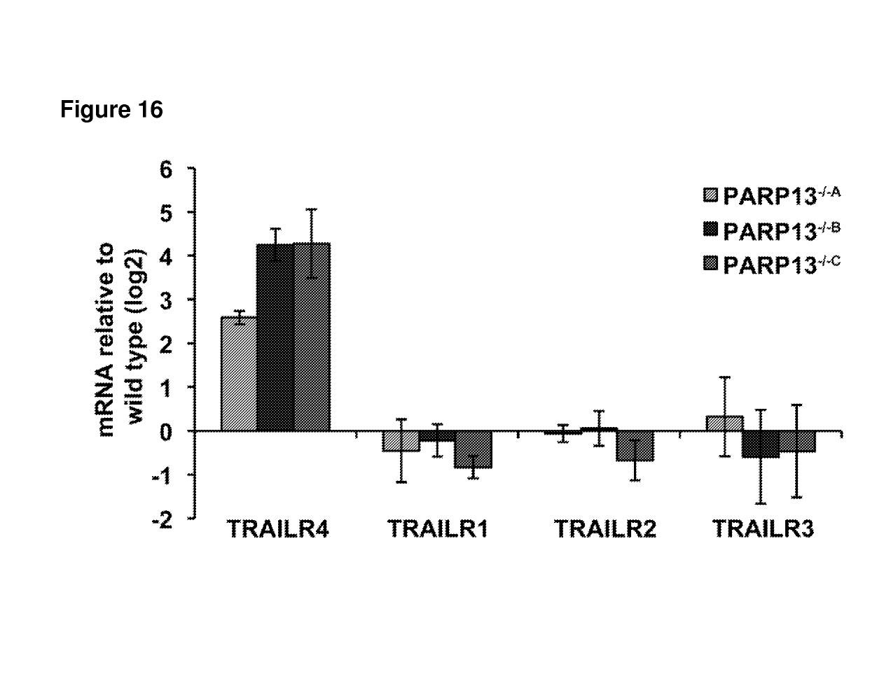

To investigate the physiological relevance of TRAILR4 regulation by PARP13 TRAIL induced apoptotic signaling upon PARP13 depletion was examined. TRAILR4 expression levels are a key regulator of TRAIL sensitivity in certain cancers (Degli-Esposti et al., Immunity 7:813-820 (1997); Morizot et al., Cell death and differentiation 10:66-75 (2003)). HeLa cells are TRAIL sensitive due to low TRAILR4 expression and exogenous expression of TRAILR4 is sufficient to confer TRAIL resistance (Merino et al., Molecular and cellular biology 26:7046-7055 (2006); Morizot et al., Cell death and differentiation 10:66-75 (2003)) (FIG. 7A). Of the four TRAIL receptors, only TRAILR4 expression is regulated by PARP13-TRAILR4 mRNA expression, examined by qRT-PCR, and protein levels, assayed by immunoblot, were increased in PARP13^{-/-} relative to wild type cells, whereas no differences in protein and mRNA levels of TRAILR1-R2 were identified between PARP13^{-/-} and wild type cells, and TRAIL-R3 protein could not be detected in this cell type, consistent with previous reports (FIGS. 7B and 16) (Merino et al., Molecular and cellular biology 26:7046-7055 (2006)). These results suggest that by modulating TRAILR4 expression PARP13 could directly regulate the cellular response to TRAIL. This possibility was examined by assaying TRAIL induced apoptosis upon PARP13 depletion in TRAIL sensitive HCT116, SW480 and HeLa cells. Consistent with the increase in TRAILR4 mRNA levels (FIG. 4D), PARP13 knockdown resulted in a pronounced resistance to TRAIL treatment in each of these cell types identifying PARP13 as a key regulator of the TRAIL response in these cell lines (FIG. 7C). The newly acquired TRAIL resistance was a specific result of increased TRAILR4 expression upon PARP13 knockdown since simultaneous knockdown of PARP13 and TRAILR4 in HeLa cells resulted in wild type TRAILR4 mRNA levels and TRAIL sensitivity profiles similar to control knockdowns (FIGS. 7C and 7D).

The TRAIL resistance conferred by PARP13 inhibition can be permanently acquired. PARP13^{-/-} cells were resistant to both short-term (24 h, FIGS. 7E and 7F) and long term TRAIL treatment (7 days, FIG. 7G), suggesting that one mechanism of TRAIL resistance in cancers could be inhibition of PARP13 function. TRAIL resistance in PARP13^{-/-} cells was completely reversed by expression of PARP13.1 but not PARP13.1^{VYFHR} or PARP13.1^{ΔZnF}, suggesting that the TRAIL resistance in these cells results from the lack of TRAILR4 mRNA regulation by PARP13 (FIGS. 7H and 7I). Together these results suggest that PARP13 is necessary and sufficient to regulate the cellular response to TRAIL in cancer cells that are TRAIL sensitive in a manner dependent on TRAILR4 expression.

### Example 9

### PARP13 Depletion Abrogates DISC Assembly and Function

TRAILR4 expression levels are important for TRAIL sensitivity in certain cancers due to the receptor's ability to sequester TRAIL from TRAILR1 and R2 binding resulting in decreased DISC assembly and apoptotic signaling at these receptors upon TRAIL treatment. This apoptotic signaling is mediated by caspase-8, which is recruited to the DISC where it is activated and autoprocesses itself. Thus caspase-8 cleavage can be used to directly report on caspase-8 enzymatic activity. To determine if the TRAIL resistance observed in PARP13^{-/-} cells results from attenuated apoptotic signaling at the TRAIL receptor level, time-dependent caspase-8 processing was analyzed in wild type or PARP13^{-/-A} cells treated with TRAIL. Whereas caspase-8 was processed in HeLa cells resulting in the appearance of p43/p41 and p18 fragments, no such processing was observed in PARP13^{-/-} cells, demonstrating an ablation of DISC signaling (FIG. 8A). A consistent upregulation of both PARP13.1 and PARP13.2 was observed upon TRAIL treatment suggesting positive feedback signaling (FIG. 8A). To determine if DISC assembly itself is defective in PARP13^{-/-} cells we compared assembly to wild type cells using standard DISC precipitation assays that utilize epitope tagged TRAIL (Walczak et al., Methods in molecular biology 414:221-239 (2008)). Recruitment of TRAILR1, TRAILR2 and caspase-8 to the DISC was greatly diminished in PARP13^{-/-} cells relative to wild type (FIG. 8B). Together these results suggest that the TRAIL resistance found upon PARP13 depletion is due to defective DISC assembly, decreased caspase-8 activation and decreased apoptotic signaling from TRAIL receptors.

### OTHER EMBODIMENTS

From the foregoing description, it will be apparent that variations and modifications may be made to the invention described herein to adopt it to various usages and conditions.

All publications, patents, and patent applications mentioned in this specification are herein incorporated by reference to the same extent as if each individual publication, patent, and patent application was specifically and individually indicated to be incorporated by reference.

What is claimed is:

1. A method of treating or decreasing the likelihood of developing a disorder associated with immune misregulation, a viral disorder, or a virus-associated disorder in a subject, the method comprising administering to the subject a therapeutically effective amount of a composition comprising an activator of a CCCH zinc finger-containing PARP, thereby treating or decreasing the likelihood of developing the disorder associated with immune misregulation, the viral disorder, or the virus-associated disorder in the subject.

2. The method of claim 1, wherein the disorder associated with immune misregulation is an autoimmune disorder, wherein the autoimmune disorder is selected from the group consisting of systemic lupus erythematosus (SLE), CREST syndrome (calcinosis, Raynaud's syndrome, esophageal dysmotility, sclerodactyl, and telangiectasia), opsoclonus, inflammatory myopathy, systemic scleroderma, primary biliary cirrhosis, celiac disease, dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, Lambert-Eaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and Neuromyelitis optica.

3. The method of claim 1, wherein the viral disorder or the virus-associated disorder is selected from the group consisting of infections due to the herpes family of viruses such as EBV, CMV, HSV I, HSV II, VZV and Kaposi's-associated human herpes virus (type 8), human T cell or B cell leukemia and lymphoma viruses, adenovirus infections, hepatitis virus infections, pox virus infections, papilloma virus infections, polyoma virus infections, infections due to retroviruses such as the HTLV and HIV viruses, Burkitt's lymphoma, and EBV-induced malignancies.

4. The method of claim 1, wherein the composition comprising the activator of a CCCH zinc finger-containing PARP is formulated for improved cell permeability.

5. The method of claim 4, wherein the activator of a CCCH zinc finger-containing PARP is iso-ADP-ribose, poly-ADP-ribose, or a derivative thereof.

6. The method of claim 1, wherein the composition is administered in combination with a second agent.

7. The method of claim 6, wherein the second agent is an immunosuppressant selected from the group consisting of: a calcineurin inhibitor, cyclosporine G tacrolimus, a mTor inhibitor, temsirolimus, zotarolimus, everolimus, fingolimod, myriocin, alemtuzumab, rituximab, an anti-CD4 monoclonal antibody, an anti-LFA1 monoclonal antibody, an anti-LFA3 monoclonal antibody, an anti-CD45 antibody, an anti-CD19 antibody, monabatacept, belatacept, azathioprine, lymphocyte immune globulin and anti-thymocyte globulin [equine], mycophenolate mofetil, mycophenolate sodium, daclizumab, basiliximab, cyclophosphamide, prednisone, prednisolone, leflunomide, FK778, FK779, 15-deoxyspergualin, busulfan, fludarabine, methotrexate, 6-mercaptopurine, 15-deoxyspergualin, LF15-0195, bredinin, brequinar, and muromonab-CD3.

8. The method of claim 6, wherein the second agent is an antiviral agent selected from the group consisting of an interferon, an amino acid analog, a nucleoside analog, an integrase inhibitor, a protease inhibitor, a polymerase inhibitor, and a transcriptase inhibitor.

9. The method of claim 1, wherein the administering results in a modulation of an interaction between a CCCH zinc finger-containing PARP and an RNA.

10. The method of claim 9, wherein the modulation is an increase in binding of the CCCH zinc finger-containing PARP to the RNA.

11. The method of claim 10, wherein the increase in binding results in a decrease in expression or activity of a gene encoded by the RNA.

12. The method of claim 11, wherein the gene encoded by the RNA is selected from any one of the genes listed in Tables 2, 4, or 6.

13. The method of claim 12, wherein the gene encoded by the RNA is selected from any one of the genes listed in Table 4.

14. The method of claim 10, wherein the increase in binding results in an increase in expression or activity of a gene encoded by the RNA.

15. The method of claim 14, wherein the gene encoded by the RNA is selected from any one of the genes listed in Table 1, 3, or 5.

16. The method of claim 15, wherein the gene encoded by the RNA is selected from any one of the genes listed in Table 3.

17. The method of any one of claims 1-16, wherein the CCCH zinc finger-containing PARP is a multiple tandem CCCH zinc finger-containing PARP.

18. The method of claim 17, wherein the multiple tandem CCCH zinc finger-containing PARP is a PARP12 or a PARP13.

19. The method of claim 18, wherein the PARP13 is PARP13.1.

20. A method of treating a TRAIL-resistant disorder in a subject, the method comprising administering to the subject a composition comprising an activator of a CCCH zinc finger-containing PARP in a therapeutically effective amount to treat the TRAIL-resistant disorder in the subject.

21. The method of claim 20, wherein the TRAIL-resistant disorder is a cancer selected from the group consisting of colon adenocarcinoma, esophagas adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, lymphoma, and non-Hodgkin's lymphoma.

22. The method of claim 20, wherein the composition comprising the activator of a CCCH zinc finger-containing PARP is formulated for improved cell permeability.

23. The method of claim 22, wherein the activator of a CCCH zinc finger-containing PARP is iso-ADP-, poly-ADP-ribose, or derivatives thereof.

24. The method of claim 20, wherein the composition is administered in combination with TRAIL therapy.

25. The method of claim 24, wherein administration of the composition to the subject in need thereof sensitizes the subject to the TRAIL therapy.

26. The method of claim 20, wherein the CCCH zinc finger-containing PARP is PARP13.

27. The method of claim 26, wherein administration of the composition increases the binding of PARP13 to TRAILR4 mRNA.

28. The method of claim 27, wherein the increase binding results in suppression of TRAILR4 expression or activity.

29. A method of modulating a CCCH zinc finger-containing PARP-RNA interaction, the method comprising contacting a CCCH zinc finger-containing PARP protein or a CCCH zinc finger-containing PARP fusion protein with a CCCH zinc finger-containing PARP activator, wherein the contacting results in the modulation of the CCCH zinc finger-containing PARP-RNA interaction.

30. The method of claim 29, wherein the CCCH zinc finger-containing PARP activator is iso-ADP-ribose, poly-ADP-ribose, or a derivative thereof.

31. The method of claim 29, wherein the modulation of the CCCH zinc finger-containing PARP-RNA interaction is an increase or a decrease in binding of CCCH zinc finger-containing PARP to the RNA.

32. The method of claim 31, wherein the modulation is an increase in binding of the CCCH zinc finger-containing PARP to the RNA.

33. The method of claim 32, wherein the increase in binding results in a decrease in expression or activity of a gene encoded by the RNA.

34. The method of claim 13, wherein the gene encoded by the RNA is selected from any one of the genes listed in Tables 2, 4, or 6.

35. The method of claim 34, wherein the gene encoded by the RNA is selected from any one of the genes listed in Table 4.

36. The method of claim 32, wherein the increase in binding results in an increase in expression or activity of a gene encoded by the RNA.

37. The method of claim 36, wherein the gene encoded by the RNA is selected from any one of the genes listed in Table 1, 3, or 5.

38. The method of claim 37, wherein the gene encoded by the RNA is selected from any one of the genes listed in Table 3.

39. The method of any one of claims 29-38, wherein the CCCH zinc finger-containing PARP is a multiple tandem CCCH zinc finger-containing PARP.

40. The method of claim 39, wherein the multiple tandem CCCH zinc finger-containing PARP is a PARP12 or a PARP13.

41. The method of claim 40, wherein the PARP13 is PARP13.1.

42. The method of claim 41, wherein an increase in binding of PARP13 to an RNA results in an increase in expression or activity of a gene encoded by the RNA.

43. The method of claim 42, wherein the gene encoded by the RNA is TRAILR4.

44. A method of identifying a candidate compound useful for treating an autoimmune disorder, viral or virus-associated disorder, or a TRAIL-resistant disorder in a subject, the method comprising:
(a) contacting a PARP13 protein or fragment thereof, with a compound; and
(b) measuring the activity of the PARP13, wherein an increase in PARP13 activity in the presence of the compound identifies the compound as a candidate compound for treating the autoimmune disorder, viral or virus-associated disorder, or a TRAIL-resistant disorder.

45. The method of claim 44, wherein an increase in PARP13 activity is an increase in binding of PARP13 to a RNA encoding a gene listed in any one of Tables 1-6.

46. The method of claim 45, wherein the gene encoded by the RNA is TRAILR4.

47. The method of claim 45, wherein the increase in binding of PARP13 to the RNA results in an increase or decrease in expression or activity of the gene encoded by the RNA.

48. The method of claim 44, wherein the compound is selected from a chemical library, or wherein the compound is an RNA aptamer, or wherein the compound is a small molecule.


SEQUENCE LISTING (42 sequences)

SEQ ID NO: 1 - DNA, 2712 bp, Homo sapiens
gccatggcggacccggaggtgtgctgcttcatcaccaaaatcctgtgcgcccacgggggccgcatggccctggacgcgctgctccaggagatcgcgctgtctgagccgcagctctgtgaggtgctgcaggtggccgggcccgaccgctttgtggtgttggagaccggcggcgaggccgggatcacccgatcggtggtggccaccactcgagcccgggtctgccgtcgcaagtactgccagagaccctgcgataacctgcatctctgcaaactcaacttgctgggccggtgcaactattcgcagtccgagcggaatttatgcaaatattctcatgaggttctctcagaagagaacttcaaagtcctgaaaaatcacgaactctctggactgaacaaagaggaattagcagtgctcctcctccaaagtgatcctttttttatgcccgagatatgcaaaagttataagggagagggtcggcagcagatttgtaaccagcagccaccgtgttcaagactccacatctgtgaccacttcacccgagggaactgtcgttttcccaactgcctccggtcccataacctgatggacagaaaggtgctggccatcatgagggagcacgggctgaaccccgacgtggtccagaacatccaggacatctgcaacagcaagcacatgcagaagaatcccccagggcccagagctccttcttcacatcgtagaaacatggcatatagggctagaagcaagagtagagatcggttctttcagggcagccaagaatttcttgcgtctgcttcagcgtctgctgagaggtcctgcacacctagtccagatcagatcagccacagggcttccctggaggacgcgcctgtggacgatctcacccgcaagttcacgtatctggggagtcaggatcgcgctcggcctccctcaggctcgtccaaggctactgatcttggaggaacaagtcaggccgggacaagccagaggtttttagagaacggcagtcaagaggacctcttgcatggaaatccaggcagcacttaccttgcttccaattcaacatcagcccccaactggaagagcctcacatcctggacgaatgaccaaggcgccaggagaaagactgtgttttctcccacgctacctgccgcccgctcttctcttggctctctgcaaacacctgaagctgtgaccaccagaaagggcacaggcttgctttcctcagactacaggatcatcaatggcaaaagtggaactcaggacatccagcctggccctctttttaataataatgctgatggagtggccacagatataacttctaccagatccttaaattacaaaagcactagcagcggtcacagagaaatatcatcacctaggattcaggatgctggacctgcttcccgagatgtccaggccactggcagaatcgcagatgatgctgacccaagagtagcacttgttaacgattctttatctgatgtcacaagtaccacatcttctagggtggatgatcatgactcagaggaaatttgtcttgaccatctgtgtaagggttgtccgcttaatggtagctgcagcaaagtccacttccatctgccttaccggtggcagatgcttattggtaaaacctggacggactttgagcacatggagacgatcgagaaaggctactgtaaccccggaatccacctctgttctgtaggaagttatacaatcaattttcgggtaatgagttgtgattcctttcccatccgacgcctctccactccttcttctgtcaccaagccagccaattctgtcttcaccaccaaatggatttggtattggaagaatgaatctggcacatggattcagtatggagaagagaaagacaaacggaaaaattcaaacgtcgactcttcatacctggagtctctctatcaatcctgtccgaggggagttgtgccatttcaggcgggctcacggaactatgagctgagtttccaagggatgattcagacaaacatagcttccaaaactcaaaaggatgtcatcagaagaccaacatttgtgcctcagtggtatgtgcagcagatgaagagagggccagaccatcagccagcaaagacctcgtcagtgtctttaactgcgacctttcgtcctcaggaggacttttgcttcctatcctcaaagaaatataagttgtcagagatccatcacctacatccagaatatgtcagagtaagtgagcattttaaagcttccatgaaaaatttcaagattgaaaagataaagaagatcgagaactcagagctcctggataaatttacatggaagaaatcgcagatgaaggaagaaggaaaactcctattttatgcgacaagccgtgcctatgtggaatctatctgttcgaataattttgacagtttcctacatgaaactcatgaaaacaaatacggaaaaggaatttactttgcaaaagatgccatctattcccacaaaaattgcccgtatgatgccaaaaacgtcgttatgtttgtagcccaagttctggttggaaagtttactgaaggaaatataacgtacacgagccctcctccacagttcgacagctgtgtggataccagatcgaatccctccgtttttgtcatctttcagaaagatcaggtttacccacaatatgtgattgaatatactgaagacaaagcctgcgtgattagttag

SEQ ID NO: 2 - DNA, 2103 bp, Homo sapiens
gccatggcggacccggaggtgtgctgcttcatcaccaaaatcctgtgcgcccacgggggccgcatggccctggacgcgctgctccaggagatcgcgctgtctgagccgcagctctgtgaggtgctgcaggtggccgggcccgaccgctttgtggtgttggagaccggcggcgaggccgggatcacccgatcggtggtggccaccactcgagcccgggtctgccgtcgcaagtactgccagagaccctgcgataacctgcatctctgcaaactcaacttgctgggccggtgcaactattcgcagtccgagcggaatttatgcaaatattctcatgaggttctctcagaagagaacttcaaagtcctgaaaaatcacgaactctctggactgaacaaagaggaattagcagtgctcctcctccaaagtgatcctttttttatgcccgagatatgcaaaagttataagggagagggtcggcagcagatttgtaaccagcagccaccgtgttcaagactccacatctgtgaccacttcacccgagggaactgtcgttttcccaactgcctccggtcccataacctgatggacagaaaggtgctggccatcatgagggagcacgggctgaaccccgacgtggtccagaacatccaggacatctgcaacagcaagcacatgcagaagaatcccccagggcccagagctccttcttcacatcgtagaaacatggcatatagggctagaagcaagagtagagatcggttctttcagggcagccaagaatttcttgcgtctgcttcagcgtctgctgagaggtcctgcacacctagtccagatcagatcagccacagggcttccctggaggacgcgcctgtggacgatctcacccgcaagttcacgtatctggggagtcaggatcgcgctcggcctccctcaggctcgtccaaggctactgatcttggaggaacaagtcaggccgggacaagccagaggtttttagagaacggcagtcaagaggacctcttgcatggaaatccaggcagcacttaccttgcttccaattcaacatcagcccccaactggaagagcctcacatcctggacgaatgaccaaggcgccaggagaaagactgtgttttctcccacgctacctgccgcccgctcttctcttggctctctgcaaacacctgaagctgtgaccaccagaaagggcacaggcttgctttcctcagactacaggatcatcaatggcaaaagtggaactcaggacatccagcctggccctctttttaataataatgctgatggagtggccacagatataacttctaccagatccttaaattacaaaagcactagcagcggtcacagagaaatatcatcacctaggattcaggatgctggacctgcttcccgagatgtccaggccactggcagaatcgcagatgatgctgacccaagagtagcacttgttaacgattctttatctgatgtcacaagtaccacatcttctagggtggatgatcatgactcagaggaaatttgtcttgaccatctgtgtaagggttgtccgcttaatggtagctgcagcaaagtccacttccatctgccttaccggtggcagatgcttattggtaaaacctggacggactttgagcacatggagacgatcgagaaaggctactgtaaccccggaatccacctctgttctgtaggaagttatacaatcaattttcgggtaatgagttgtgattcctttcccatccgacgcctctccactccttcttctgtcaccaagccagccaattctgtcttcaccaccaaatggatttggtattggaagaatgaatctggcacatggattcagtatggagaagagaaagacaaacggaaaaattcaaacgtcgactcttcatacctggagtctctctatcaatcctgtccgaggggagttgtgccatttcaggcgggctcacggaactatgagctgagtttccaagggatgattcagacaaacatagcttccaaaactcaaaaggatgtcatcagaagaccaacatttgtgcctcagtggtatgtgcagcagatgaagagagggccagagtaa

SEQ ID NO: 3 - DNA, 2109 bp, Homo sapiens
gccatggcccaggccggcgtcgtcggtgaggtcacccaggtgctgtgcgcggccgggggcgccctggagttgcccgagctgcggcgccgcttgcggatgggcttgagcgccgacgcgctggagcggctgctgcggcagcgtgggcgcttcgtggtggcggtgcgggcgggcggcgcagccgcggccccggagcgcgtggtgctggccgcctcgccgctgcgcctgtgtcgcgcgcaccagggctccaagccgggctgcgtggggctctgcgcgcagctccacctctgcaggttcatggtctacggcgcctgcaagttcctgagagccgggaagaactgtaggaatagtcacagcttgacaaccgaacacaacctgagtgtgctgagaactcatggcgttgaccacctgagctataatgagctatgccaactcttgtttcagaacgacccctggcttttgccagaaatttgccaacattacaacaaaggagatggaccccacggctcttgtgcctttcaaaagcagtgcatcaagctccatatctgccagtattttttacagggggaatgcaagtttggcactagctgtaagagatcccatgatttctctaattctgagaatctggaaaaattggagaagttgggtatgagctcagacctggtgagcaggctgcctaccatttatagaaatgcacatgacatcaagaataagagctctgcccccagcagagtgcctcctctttttgtcccacaggggacttctgaaagaaaagacagttcaggttctgtgtccccaaacactcttagccaggaggagggtgatcagatctgtttgtaccatatccggaaaagttgtagctttcaagataagtgccatagagttcatttccatttgccgtatcgatggcaattcttggatagaggcaaatgggaggatttggacaacatggaacttattgaagaggcatattgcaatcccaaaatagaaaggatcctgtgctctgagtcagccagtacctttcactctcattgtctgaactttaacgccatgacttacggtgctacccaggctcgccgcctctccacggcctcctctgtcaccaaacctccacacttcatcctcaccactgactggatttggtactggagtgatgagtttggttcttggcaggaatatggaagacagggcacggtgcaccctgtgaccactgtcagcagtagcgacgtggagaaggcctacctggcctactgtacaccggggtctgacggccaggcagccaccttgaagttccaggccggaaagcacaactacgagttagatttcaaagccttcgttcagaaaaacctggtctatggcacaactaaaaaggtttgccgcagacccaaatacgtgtctccccaggatgtgacgaccatgcaaacctgcaataccaagtttccaggcccgaagagcatcccagactattgggactcctctgccctgccagacccaggctttcagaagatcacccttagttcttcctcggaagagtatcagaaggtctggaacctctttaaccgcacgctgcctttctactttgttcagaagattgagcgagtacagaacctggccctctgggaagtctaccagtggcaaaaaggacagatgcagaagcagaatggagggaaggccgtggacgagcggcagctgttccacggcaccagcgccatttttgtggacgccatctgccagcagaactttgactggcgggtctgtggtgttcatggcacttcctacggcaaggggagctactttgcccgagatgctgcatattcccaccactacagcaaatccgacacgcagacccacacgatgttcctggcccgggtgctggtgggcgagttcgtcaggggcaatgcgtcctttgtccgtccgccggccaaggagggctggagcaacgccttctatgatagctgcgtgaacagtgtgtccgacccctccatctttgtgatctttgagaaacaccaggtctacccagagtatgtcatccagtacaccacctcctccaagccctcggtcacaccctccatcctgctggccttgggctccctgttcagcagccgacagtga

SEQ ID NO: 4 - DNA, 1977 bp, Homo sapiens
attatggaaatggaaaccaccgaacctgagccagactgtgtagtgcagcctccctctcctcctgatgacttttcatgccaaatgagactctctgagaagatcactccattgaagacttgttttaagaaaaaggatcagaaaagattgggaactggaaccctgaggtctttgaggccaatattaaacactcttctagaatctggctcacttgatggggtttttagatctaggaaccagagtacagatgagaacagcttacatgaacctatgatgaagaaagccatggaaatcaattcatcatgcccaccagcagaaaataatatgtctgttctgattcctgataggacaaatgttggggaccagataccggaagcccatccttccactgaagctccagaacgagtggttccaatccaagatcacagctttccatcagaaaccctcagtgggacggtggcagattccacaccagctcacttccagactgatcttttgcacccagtttcaagtgatgttcctactagtcctgactgcttagacaaagtcatagattatgttccaggcattttccaagaaaacagttttacaatccaatacattctggacaccagtgataagctgagtactgagctctttcaggacaaaagtgaagaggcttcccttgacctcgtgtttgagctggtgaaccagttgcagtaccacactcaccaagagaacggaattgaaatttgcatggactttctgcaaggcacttgtatttatggcagggattgtttgaagcaccacactgtcttgccatatcattggcagatcaaaaggacaactactcaaaagtggcagagtgtattcaatgattctcaggagcacttggaaagattttactgtaacccagaaaatgatagaatgagaatgaagtatggaggacaagaattttgggcagatttgaatgccatgaacgtgtatgaaacaactgaatttgaccaactacgaaggctgtccacaccaccctctagcaatgtcaactctatttaccacacagtctggaaattcttctgtagggaccactttggatggagagagtatcccgagtctgtcattcgattgattgaagaagccaactctcggggtctgaaagaggttcgatttatgatgtggaataaccactacatcctccacaattcattcttcaggagagagataaaaaggagacccctcttccgctcctgttttatactgcttccatatttacagacacttggtggggttcccacacaagctcctccacctcttgaagcaacttcatcatcacaaattatctgcccagatggggtcacttcagcaaacttttaccctgaaacttgggtttatatgcatccatctcaggacttcatccaagtccctgtttctgcagaggataaaagttatcggatcatttacaatctttttcataagactgtgcctgagtttaaatacagaattttgcagatattgagagtccaaaaccagtttctttgggagaaatataaaaggaaaaaggaatatatgaacaggaaaatgtttggccgtgacaggataataaatgagagacatttatttcatggaacatcccaggatgtggtagatggaatctgcaaacacaactttgaccctcgagtctgtggaaagcatgctacaatgtttggacaaggcagttattttgcaaagaaggcaagctactctcataacttttctaagaagtcctccaaaggagtccacttcatgtttctggccaaagtgctgacgggcagatacacaatgggcagtcatggcatgagaaggcccccgccagtcaatcctggcagtgtcaccagtgacctttatgactcttgtgtggataatttctttgagcctcagatttttgtcatttttaatgatgaccagagttacccttattttgttatccaatatgaagaagtcagtaacactgtttccatttga

SEQ ID NO: 5 - RNA, 25 nt, Artificial Sequence
  Synthetic Construct
gcucacggaacuaugagcugaguuu

SEQ ID NO: 6 - DNA, 21 bp, Artificial Sequence
  Synthetic Construct
gaauuuacuuugcaaaagatt

SEQ ID NO: 7 - DNA, 20 bp, Artificial Sequence
  Synthetic Construct
ttaagttcgtcgtcttcatc

SEQ ID NO: 8 - DNA, 20 bp, Artificial Sequence
  Synthetic Construct
tctgatctatgagatcctgc

SEQ ID NO: 9 - DNA, 19 bp, Artificial Sequence
  Synthetic Construct
aagtctctaggttctgagc

SEQ ID NO: 10 - DNA, 21 bp, Artificial Sequence
  Synthetic Construct
ttttatggttgcattgagaac

SEQ ID NO: 11 - DNA, 21 bp, Artificial Sequence
  Synthetic Construct
accatgagtgagaacaataag

SEQ ID NO: 12 - DNA, 20 bp, Artificial Sequence
  Synthetic Construct
ttagataggccagtaggttg

SEQ ID NO: 13 - DNA, 20 bp, Artificial Sequence
  Synthetic Construct
gtaccagcagtatgtgaaag

SEQ ID NO: 14 - DNA, 20 bp, Artificial Sequence
  Synthetic Construct
atggttagaagttcaagagc

SEQ ID NO: 15 - DNA, 20 bp, Artificial Sequence
  Synthetic Construct
tccagattgtccgcaactac

SEQ ID NO: 16 - DNA, 23 bp, Artificial Sequence
  Synthetic Construct
cttcttagctccctcgacaatag

SEQ ID NO: 17 - DNA, 19 bp, Artificial Sequence
  Synthetic Construct
acatccaagcaagaaaagg

SEQ ID NO: 18 - DNA, 19 bp, Artificial Sequence
  Synthetic Construct
ctcagtgtctgagtagagc

SEQ ID NO: 19 - DNA, 18 bp, Artificial Sequence
  Synthetic Construct
ccactgagactctgatgc

SEQ ID NO: 20 - DNA, 20 bp, Artificial Sequence
  Synthetic Construct
cagttttgttgacccatttc

SEQ ID NO: 21 - DNA, 19 bp, Artificial Sequence
  Synthetic Construct
cactgagactctgagacag

SEQ ID NO: 22 - DNA, 23 bp, Artificial Sequence
  Synthetic Construct
gctttagccagccacctttatct

SEQ ID NO: 23 - DNA, 19 bp, Artificial Sequence
  Synthetic Construct
gtccagcaggatctcatag

SEQ ID NO: 24 - DNA, 21 bp, Artificial Sequence
  Synthetic Construct
aagaaggttcattgttggaag

SEQ ID NO: 25 - DNA, 20 bp, Artificial Sequence
  Synthetic Construct
gacgacatggagaaaatctg

SEQ ID NO: 26 - DNA, 20 bp, Artificial Sequence
  Synthetic Construct
atgatctgggtcatcttctc

SEQ ID NO: 27 - DNA, 20 bp, Artificial Sequence
  Synthetic Construct
gacttgagtgggctctttgt

SEQ ID NO: 28 - DNA, 22 bp, Artificial Sequence
  Synthetic Construct
tatctgttactcagggtctcgt

SEQ ID NO: 29 - DNA, 22 bp, Artificial Sequence
  Synthetic Construct
cttggagcattgagaccctaaa

SEQ ID NO: 30 - DNA, 23 bp, Artificial Sequence
  Synthetic Construct
gtttcctttgcatgtccttcttc

SEQ ID NO: 31 - DNA, 18 bp, Artificial Sequence
  Synthetic Construct
atgagtgaggtcaccaag

SEQ ID NO: 32 - DNA, 20 bp, Artificial Sequence
  Synthetic Construct
ccttgaataagttccaggtg

SEQ ID NO: 33 - DNA, 20 bp, Artificial Sequence
  Synthetic Construct
ctgtaaacaggtggaaaagg

SEQ ID NO: 34 - DNA, 22 bp, Artificial Sequence
  Synthetic Construct
gtatctcctaatcgcaaaagag

SEQ ID NO: 35 - DNA, 15 bp, Artificial Sequence
  Synthetic Construct
cttttgcgtcgccag

SEQ ID NO: 36 - DNA, 20 bp, Artificial Sequence
  Synthetic Construct
ttgatggcaacaatatccac

SEQ ID NO: 37 - DNA, 54 bp, Artificial Sequence
  Synthetic Construct
aaatttaaataatacgactcactatagggaagaatctcttcaggaaaccagagc

SEQ ID NO: 38 - DNA, 42 bp, Artificial Sequence
  Synthetic Construct
ttttttttttttttttttttgtagttagatgtcattaaatgc

SEQ ID NO: 39 - DNA, 54 bp, Artificial Sequence
  Synthetic Construct
aaatttaaataatacgactcactatagggagatgccgcacagccacaatgcttt

SEQ ID NO: 40 - DNA, 43 bp, Artificial Sequence
  Synthetic Construct
ttttttttttttttttttttaatcctgggcatatgtaaacatc

SEQ ID NO: 41 - RNA, 25 nt, Artificial Sequence
  Synthetic Construct
aaacucagcucauaguuccgugagc

SEQ ID NO: 42 - DNA, 21 bp, Artificial Sequence
  Synthetic Construct
ucuuuugcaaaguaaauucct